(19) United States
(12) Patent Application Publication
(10) Pub. No.: US 2024/0016643 A1
(43) Pub. Date: Jan. 18, 2024

(54) EXTERNAL ANKLE BRACE

(71) Applicant: TayCo Brace, LLC, South Bend, IN (US)

(72) Inventors: Frederick John FERLIC, South Bend, IN (US); Mike BEAN, South Bend, IN (US); Nate LAZENGA, South Bend, IN (US)

(21) Appl. No.: 18/372,363

(22) Filed: Sep. 25, 2023

Related U.S. Application Data
(63) Continuation of application No. PCT/US2022/022018, filed on Mar. 25, 2022.
(60) Provisional application No. 63/251,584, filed on Oct. 1, 2021.
(60) Provisional application No. 63/247,266, filed on Sep. 22, 2021.
(60) Provisional application No. 63/165,784, filed on Mar. 25, 2021.

(51) Int. Cl. A61F 5/01

(52) U.S. Cl. CPC A61F 5/0111; A61F 2005/0174; A61F 2250/001; A61F 2005/0137; A61F 2005/0179; A61F 2005/0158; A61F 2005/0167

(57) ABSTRACT

An external ankle brace is disposed on the exterior of a shoe having a heel portion, a toe portion, a sole, and oppositely disposed sides. The external ankle brace comprises a rigid heel enclosure having a rear portion, configured to receive the heel of the shoe, and a forward portion having a medial sidewall and a lateral sidewall for collectively and concurrently at least partially encircling the sides of the shoe. A lateral upright extension is present. A medial upright extension is present. A lower fastening system comprises at least one lower adjustable connecting strap for extending underneath the sole of the shoe. An upper fastening system comprises at least one upper connecting strap for selectively connecting the lateral sidewall to the medial sidewall across the top of the shoe.

### CROSS-REFERENCE TO RELATED APPLICATIONS

This application claims priority to PCT Patent Application No. PCT/US2022/022018, filed Mar. 25, 2022, which claims priority to U.S. Provisional Patent Application No. 63/165,784, filed on Mar. 25, 2021, and U.S. Provisional Patent Application No. 63/247,266, Filed on Sep. 22, 2021, and U.S. Provisional Patent Application No. 63/251,584, filed on Oct. 1, 2021, the entirety of each of which is hereby incorporated by reference for all purposes.

### TECHNICAL FIELD

The disclosure pertains generally to preventative and rehabilitative equipment, and more particularly to an ankle brace.

### BACKGROUND

In the world of sports, ankle injuries are among the most common cause of lost playing time in a sporting career, with a typical ankle injury leaving the athlete out of competition for up to a month. Ankle sprains occur when there is a rapid shifting of weight from one direction to another. The force generated from the movement causes the foot to roll either inwards, which is known as inversion rotation; or outwards, which is known as eversion rotation. Both the inversion and eversion motion of the ankle cause the ligaments on the outside of the ankle to stretch or tear depending on the force that was generated during the movement.

Current braces vary from woven fabric that acts as a glove and wraps around the ankle, to rigid plastic uprights that are strapped around the ankle. The woven fabric braces typically are made of a thin fabric that envelope the ankle and are laced together to support the ankle from both inversion and eversion rotation. The main drawback with these types of braces is that the material lacks the resistance to prevent the ankle from rolling under intense forces. Further, fabric braces also have to be worn within the shoe, which causes the shoe to fit tighter or, in some cases, forces the user to move up a shoe size in order to wear the brace. In terms of the rigid uprights braces, these braces are typically much heavier than the fabric braces and also much larger. Fitting a rigid brace into a tight shoe almost never works, which forces the user to move up to the next shoe size to accommodate for the bulkiness of the brace. When the user moves up a shoe size, the shoe is no longer sized correctly for the foot and thus loses a portion of its intended use and purpose. These braces leave the user at risk for further injury because either the brace isn't strong enough to support the ankle or the shoe isn't fitted properly to the foot.

### SUMMARY

In an aspect, an external ankle brace for selectively restricting movement of an ankle in at least one of a first direction and a rotation direction, and selectively permitting movement of the ankle in a second direction is provided. The external ankle brace is disposed on the exterior of a shoe. The shoe has a heel portion, a toe portion longitudinally spaced from the heel portion, a sole, and oppositely disposed sides. The external ankle brace comprises a rigid heel enclosure having a rear portion and a forward portion. The rear portion is configured to receive and at least partially encircle the heel portion of the shoe. The forward portion has a medial sidewall and a lateral sidewall for collectively and concurrently at least partially encircling the sides of the shoe concurrent with the rear portion connecting the medial and lateral sidewalls to collectively at least partially encircle the side, and fully encircle the heel portion, of the shoe. The lateral and medial sidewalls each extend from the rear portion toward a toe of a wearer's foot and each extend beyond a talus of the wearer's foot. A lateral upright extension is selectively perpendicular to the rigid heel enclosure and is pivotally attached to the lateral sidewall. The lateral upright extension includes a lateral reinforcing strut. A medial upright extension is selectively perpendicular to the rigid heel enclosure and is pivotally attached to the medial sidewall. The medial upright extension includes a medial reinforcing strut. A lower fastening system comprises at least one lower connecting strap for connecting the lateral sidewall to the medial sidewall and extending underneath the sole of the shoe. An upper fastening system comprises at least one upper connecting strap for selectively connecting the lateral sidewall to the medial sidewall across the top of the shoe. The upper connecting strap is located longitudinally between the lower connecting strap and the lateral and medial upright extensions.

### BRIEF DESCRIPTION OF THE DRAWINGS

For a better understanding, reference may be made to the accompanying drawings, in which:

FIG. 1 is a lateral side view showing a first aspect of the external ankle brace with an athletic shoe.

FIG. 2 is a perspective view of the external ankle brace of FIG. 1 from the medial side.

FIG. 3 is a perspective view showing the underside of the external ankle brace of FIG. 1.

FIG. 4 is a perspective view of the external ankle brace of FIG. 1 from the lateral side.

FIG. 5 is a top view of the external ankle brace of FIG. 1.

FIG. 6 is a rear view of the external ankle brace of FIG. 1.

FIG. 7 is a lateral side view showing a second aspect of the external ankle brace, in a first configuration, with an athletic shoe.

FIG. 8 is a lateral side view showing a second aspect of the external ankle brace, in a second configuration, with an athletic shoe.

FIG. 9 is a perspective rear view of a component of the external athletic braces of FIGS. 7-8.

FIG. 10 is a partial perspective front view of the component of FIG. 9.

FIG. 11 is a partial front view of the component of FIG. 9.

FIG. 12 is a side view of the component of FIG. 9.

FIG. 13 is a bottom view of the component of FIG. 9.

FIGS. 14-31 and 65A-C depict via CAD images details of a different embodiment, a third embodiment, different than the first and second embodiments of FIGS. 1-13 above, which embodiment has advantages over the embodiments of FIGS. 1-13.

FIGS. 32-39, 45-51 and 60 depict more details of the brace of the 3^{rd} embodiment;

FIGS. 40-44 provide views of a lock bar according to the third embodiment;

FIGS. 52-59 provide drawings that serve as a basis for performance related features of the third embodiment as compared to the first and second embodiment;

FIGS. 61-63H and 66-70 and 74 and 75 and 76, depict real life product according to the third embodiment;

FIGS. 64, 71, 77-80 provide an exemplary algorithm for an exemplary method;

FIGS. 72 and 73 provide comparison data;

FIGS. 81-94 provide views used to present additional comparison data; and

FIGS. 95 and 96 provide views used to show performance features.

FIGS. 96 to 111 show additional exemplary embodiments.

### DETAILED DESCRIPTION

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, the term “and/or” can include any and all combinations of one or more of the associated listed items.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure.

A “wearer” or “user”, as described herein, is a person who has the external ankle brace on his or her own foot/ankle.

Ankle injuries are among the most common cause of lost playing time in a sporting career and although there are current preventative solutions, those current braces leave the user at risk for further injury because either the brace isn't strong enough to support the ankle or the shoe isn't fitted properly to the foot since “inside the shoe” braces tend to force the user to use a bigger shoe size. In addition, outside the athletic context, ankle braces can be used by the general population while recovering from ankle injury or attempting to prevent reinjury, to wear while going about daily activities.

The present disclosure provides a rigid support and a much faster application time, all without compromising the fit of the shoe. Essentially, the external ankle brace described herein assists anyone recovering from an ankle injury and/or seeking to avoid injury to limit inversion/eversion and rotation, and can also selectively limit plantar flexion/dorsiflexion. This at least partial motion limiting can help an ankle to heal or avoid (re)injury; thus, the patient can return to function and normal daily activities (including athletic activities, as desired) without the need for specialized or temporary-use footwear (e.g., a larger shoe on the braced side). The external ankle brace of the present disclosure may provide superior stability to an internal ankle brace and improved functionality over known solutions, such as a walking boot or internal ankle foot orthoses.

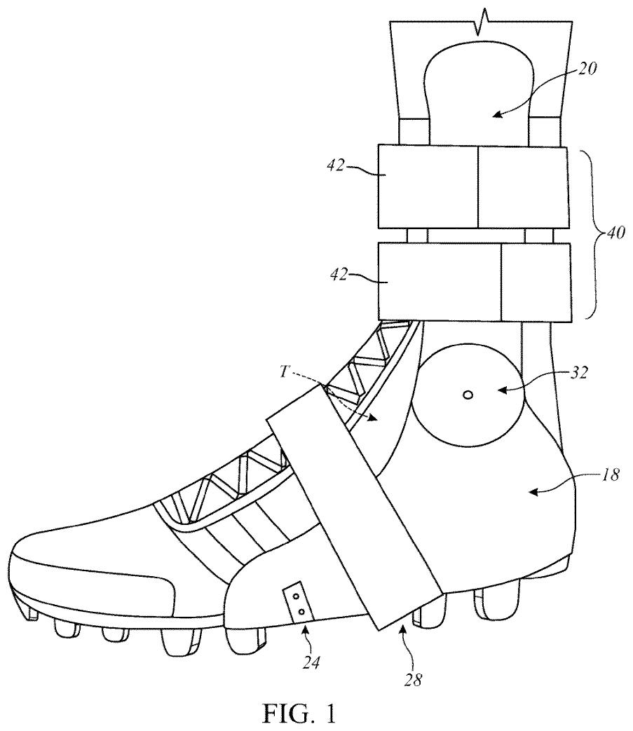

The present disclosure relates to an external ankle brace that is adapted to fit around a shoe to prevent and minimize injury to an ankle. The shoe has a heel portion, a toe portion longitudinally spaced from the heel portion, a sole, and oppositely disposed sides. The term “longitudinal” is used herein to reference a direction oriented along the foot between the heel and toe. The interaction between the external ankle brace and the shoe can be seen in FIG. 1.

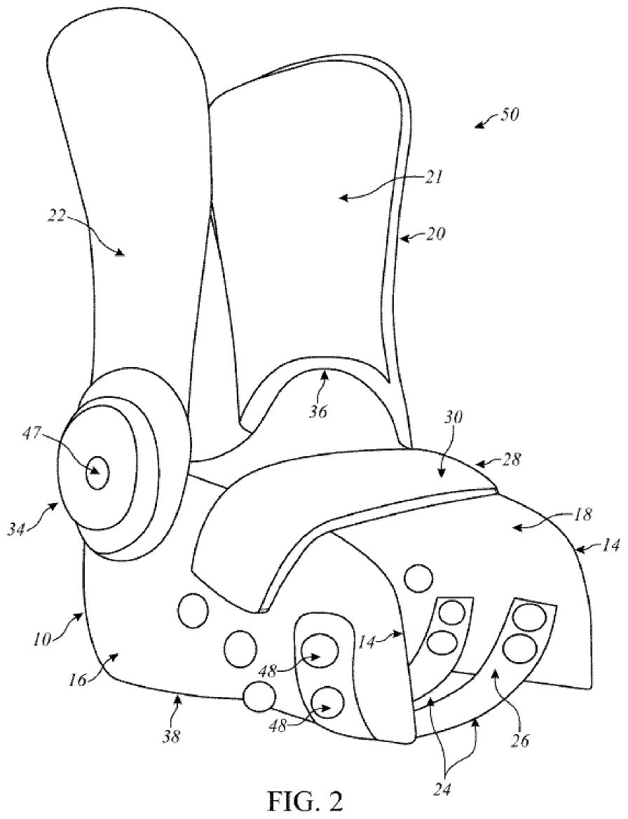

The external ankle brace of the present disclosure is generally indicated at 50 in FIG. 2. The external ankle brace 50 includes a rigid heel enclosure 10, a lateral upright extension 20, a medial upright extension 22, a lower fastening system 24, and an upper fastening system 28.

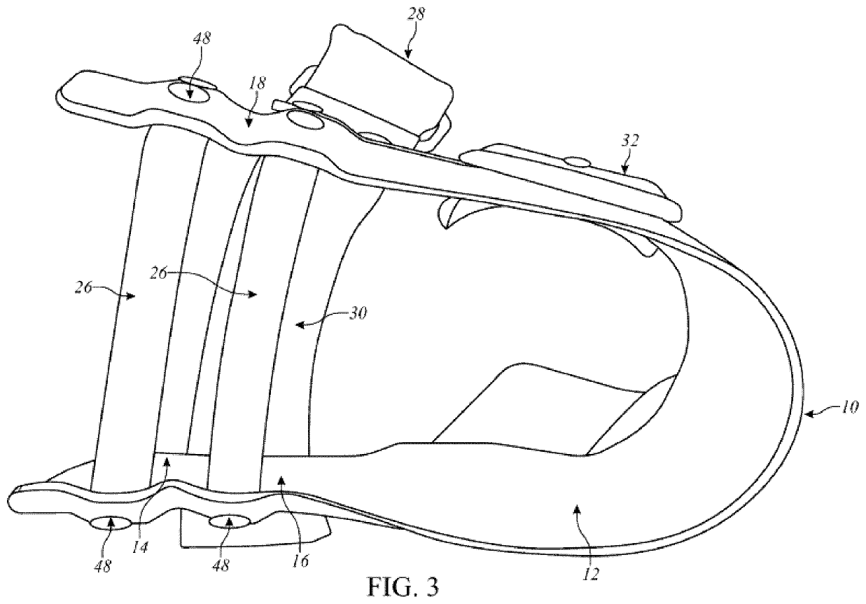

The rigid heel enclosure 10 has a rear portion 12 (FIG. 3), for receiving and at least partially encircling the heel of the shoe, and a forward portion 14, for surrounding, e.g., collectively and concurrently at least partially surrounding, the sides of the shoe. The forward portion 14 at least partially encircles the sides of the shoe concurrent with the rear portion 12 connecting the medial and lateral sidewalls 18 and 16 to collectively at least partially encircle the side, and fully encircle the heel, of the shoe. The heel enclosure 10 may be at least partially made from rigid plastic pieces and/or any other suitable material. The forward portion 14 further includes a medial sidewall 16 and a lateral sidewall 18, which may also be at least partially made from rigid plastic pieces and/or any other suitable material.

As shown in the Figures, it is contemplated that the lateral and/or medial sidewalls 16 and 18 may each extend from the rear portion 12 in a longitudinal direction toward a toe of the wearer's foot and may each extend beyond a talus (shown as “T” in FIG. 1) of the wearer's foot—i.e., may be located lateral/medial to the lace portion of the shoe. (It is recognized that a particular wearer may have nonstandard bone structure to which this “landmark” would not apply—this description presumes a foot structure which is anatomically normal, in which the talus marks the “inflection point” at which the lower leg turns into the top of the foot.) Stated differently, the rigid heel enclosure 10 has a rear portion 12, and a forward portion 14 partially located on both sides of the heel, one side of the forward portion 14 comprising the medial sidewall 16 and another side of the forward portion 14 comprising the lateral sidewall 18. Each of the medial and lateral sidewalls 16 and 18 extends at least a predetermined amount of the longitudinal distance from a pivot point on the rigid heel enclosure 12 forward from heel toward toe. The medial and lateral sidewalls 16 and 18 run beside (and thus at least partially surround) a predetermined percentage of a total longitudinal length of the user's shoe that is in the range of 5-40%, more particularly about 10-35%, and more particularly about 20-30% of the longitudinal distance between the user's ankle and the end of the user's toe. It is contemplated that an external ankle brace 50 will, for most use environments, include medial and lateral sidewalls 16 and 18 that extend beyond the talus T (as opposed to a straight “stirrup” on the sides of the ankle with little/no extension), to accommodate placement of a lower connecting strap 26 that fits under the shoe's arch gap.

The rigid heel enclosure 10 also has an upper end 36 (FIG. 2) for receiving the upright extensions 20 and 22, and a lower end 38 for surrounding the bottom of the shoe. The rigid heel enclosure 10 can be configured as desired, such as by orienting the lower end 38 in a substantially “straight” non-contoured configuration (parallel to, and fitting closely about, the base of the shoe), and/or the upper end 36 including a curved contour as shown in FIG. 2 for mechanical, manufacturing efficiency, aesthetic, and/or any other considerations.

The lateral upright extension 20 is oriented generally selectively perpendicular to at least the lateral sidewall 18 of the rigid heel enclosure 10 and is pivotally attached to the lateral sidewall 18 at the upper end 36 by a lateral ankle joint 32 (FIG. 4). The joint allows the lateral upright extension 20 to rotate during motion giving the external ankle brace a less restrictive feel compared to previous braces—thus, the lateral upright extension 20 is perpendicular to the lateral sidewall 18 during certain portions of the pivoting process, such as when the ankle is in a neutral position, in neither planar flexion nor dorsiflexion.

In other words, the lateral sidewall 18 is both substantially planar in a vertical fashion (up and down) and substantially planar in a longitudinal fashion (back of shoe to front of shoe), including some minor contours to fit the curves of a shoe and/or a user. The lateral upright extension 20 selectively pivots forward and backward to allow for plantar flexion and dorsiflexion while still providing inversion/eversion and rotational support. As desired, the lateral upright extension 20 may be configured for permanent or temporary fixation with respect to the lateral sidewall 18, which maintains perpendicularity therebetween and does not allow for plantar flexion or dorsiflexion. When the fixation screw (or screws) are removed as discussed below to reverse a temporary fixation situation, then the lateral and medial upright extensions 20 and 22 can move forward and backward to allow for plantar flexion and dorsiflexion.

The lateral upright extension 20 may be made from plastic and/or any other suitable material. The lateral ankle joint 32 includes a fastener 47 and allows the lateral upright extension 20 to rotate relative to the lateral sidewall 18. Although the current embodiment uses at least one screw as the fastener 47, one having ordinary skill in the art will appreciate that a pivot hinge, hex nut, revolving joint, Chicago screw, or any other suitable member could be used to allow the joint to pivot. As shown in FIG. 5, the lateral upright extension 20 may have a concave shape for increased comfort for the user. The lateral upright extension 20 can also include foam or other suitable padding on the interior side 21 (FIG. 2) of the lateral upright extension 20 to increase comfort and to allow a better fit for the user.

The medial upright extension 22 is oriented generally selectively perpendicular to at least the medial sidewall 16 of the rigid heel enclosure 10 and is pivotally attached to the medial sidewall 16 at the upper end 36 by a medial ankle joint 34, with the perpendicularity and pivotal properties being similar to those of the lateral upright extension 20. The medial upright extension 22 may be made of rigid plastic and/or any other suitable material. The medial ankle joint 34 has a fastener 47 and allows the medial upright extension 22 to rotate relative to the medial sidewall 16—thus, the medial upright extension 22 is perpendicular to the medial sidewall 16 during certain portions of the pivoting process.

To adjust for anatomical positioning of the ankle, the medial ankle joint 34 may be positioned somewhat closer to the upper end 36 than the position of the lateral ankle joint 32, since the medial malleolus (that bone that protrudes from the inside of the user's ankle) may be slightly higher (more cephalad) than the lateral malleolus (the bone that protrudes from the outside of the ankle), in most anatomically normal wearers. The pivot points between the lateral and medial upright extensions 20 and 22, and their respective lateral and medial sidewalls 18 and 16 on each side of the external ankle brace 50 could be desirably aligned with the medial-higher anatomy of the body, although it is contemplated that versions of the external ankle brace 50 could have equal-medial-lateral or medial-lower pivot points for any reason(s), such as, but not limited to, manufacturing considerations or the anatomy of a particular user.

In addition, the medial ankle joint 34 of the external ankle brace 50 may be offset, as desired from a 180-degree directly opposing position relative to the lateral ankle joint 32. For example, the medial ankle joint 34 may be rotated in the range of about 5-20 degrees, and more particularly about 10-15 degrees, forward (towards the toes) from a 180 degree position relative to the lateral ankle joint 32. Similar to the ankle joint height discussed immediately previously, this is anatomically correct. To clarify, imagine a circle that is parallel to the ground (on the same plane). Instead of having the medial and lateral ankle joints 34 and 32 being 180 degrees apart on that circle, the medial ankle joint 34 could be moved (and rotated to keep facing the center of the circle, if desired), some distance toward the toes along that circle because the medial malleolus is closer to the toes than is the lateral malleolus. It is contemplated that versions of the external ankle brace 50 could instead have 180-degree-spaced or lateral-forward pivot points for any reason(s), such as, but not limited to, manufacturing considerations or the anatomy of a particular user.

Although the current embodiment uses at least one screw as the fastener 47, one having ordinary skill in the art will appreciate that a pivot hinge, hex nut, revolving joint, Chicago screw, or any other suitable member could be used to allow the joint to pivot. As shown in FIG. 5, the medial upright extension 22 may have a concave shape for increased comfort for the user. The medial upright extension 22 can also include foam or other padding on the interior side 23 (FIG. 4) of the medial upright extension to increase comfort and to allow a better fit for the user.

The lower fastening system 24 has at least one lower connecting strap 26 and at least one strap fastener 48 for connecting the lateral sidewall 18 to the medial sidewall 16 (FIG. 2) and extending underneath the sole of the shoe. Although the current embodiment uses a rubber strap, one having ordinary skill in the art would appreciate that plastic, nylon, or any other suitable strap type that is commonly known in the art could be used. Similarly, although the current embodiment uses rivets to fasten the straps to each of the lateral and medial sidewalls 18 and 16 respectively, any other fastener could be used.

The upper fastening system 28 has at least one upper connecting strap 30 for selectively connecting the lateral sidewall 18 to the medial sidewall 16 while passing over the top of the shoe. The upper fastening system 28 further includes a D-ring 31 which is fixed on the lateral sidewall. A hook and loop fastener type upper connecting strap 30 is fixed to the medial sidewall and is looped through the D-ring 31 to overlaps back itself. This allows for an adjustable fastening system to accommodate various sizes without compromising support. The term “hook and loop fastener” is used herein to reference a type of fastening device such as, but not limited to, VELCRO® brand fasteners, available from Velcro USA Inc. & Velcro Group Corporation of Manchester, New Hampshire. Although the current embodiment uses a hook and loop fastener upper connecting strap 30 to removably connect the sidewalls 16 and 18 and/or adjust the tightness of their mutual connection across the wearer's instep, one having ordinary skill in the art would appreciate that any kind of removable and/or adjustable strap can be used. Similarly, although the embodiment of FIGS. 1-6 only uses one upper connecting strap 30, any number of straps can be used to removably and/or adjustably connect the sidewalls 16 and 18 over the top of the shoe, as will be discussed further below. As shown in FIGS. 1-5, the upper connecting strap 30 is located longitudinally between the lower connecting strap 26 and the lateral and medial upright extensions 20 and 22, though one of ordinary skill in the art could readily configure an external ankle brace 50 having lower connecting strap(s) 26 in a desired location to fit a particular wearer, a selected shoe model, a certain cleat spacing, or for any other reason. The term “longitudinally between” is used herein to relate to the relative positions of two or more structures in the longitudinal direction—the two or more structures need not intersect, in whole or part, the same longitudinal line as one another but could be offset along different, substantially parallel longitudinal lines.

As shown in FIG. 5, the external ankle brace 50 restricts movement of the ankle in the first directions indicated by arrows 44 (the eversion and inversion directions) and permits ankle movement in the second directions indicated by arrows 46 (the plantar flexion and dorsiflexion directions). The external ankle brace 50 also restricts rotation of the ankle (“windshield wiper” motion of the toes with a stationary heel, or vice versa.

The external ankle brace 50 may include an upright fastening system 40 (FIG. 1), which would have at least one upright connecting strap 42 for selectively connecting the lateral upright extension 20 to the medial upright extension 22 above the ankle. This upright connecting strap 42 could include hook and loop fastener or any other type of strap that would allow for an adjustable and/or removable connection.

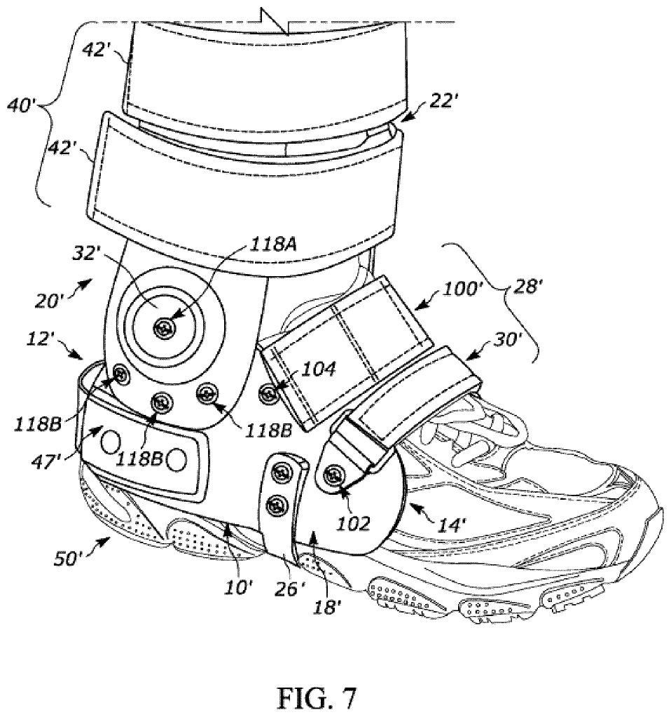

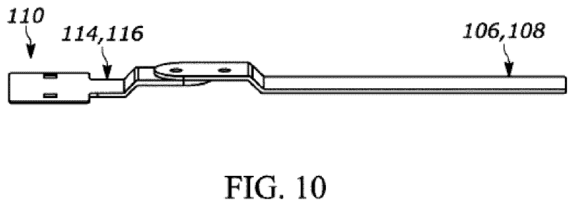

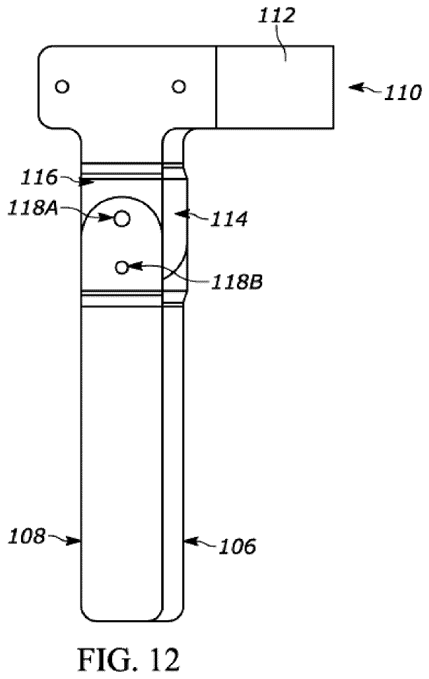

FIGS. 7-13 illustrate a second embodiment of an external ankle brace 50′. The external ankle brace 50′ of FIGS. 7-13 is similar to the external ankle brace 50 of FIGS. 1-6 and therefore, structures of FIGS. 7-13 that are the same as or similar to those described with reference to FIGS. 1-6 will be shown and/or described as having the same reference numbers with the addition of a “prime” mark. Description of common elements and operation similar to those in the previously described first embodiment will not be repeated with respect to the second embodiment or vice versa, but should instead be considered to be incorporated below/above by reference as appropriate.

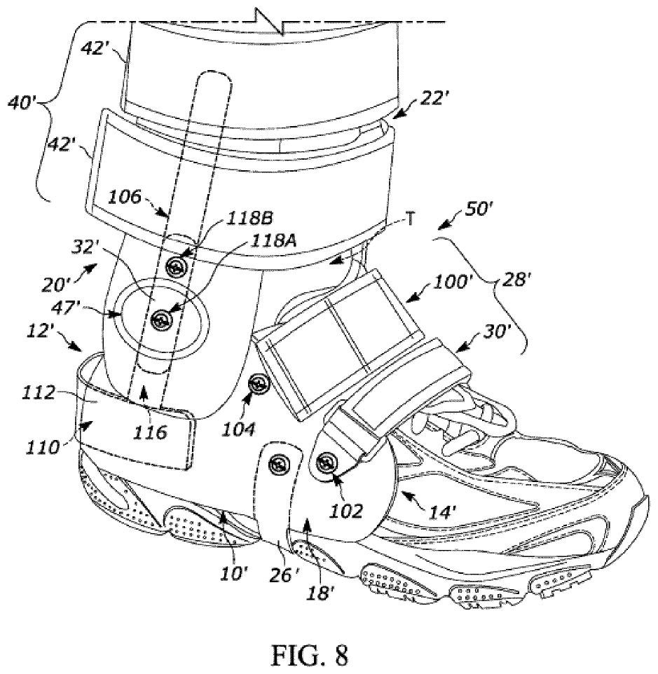

As shown in FIGS. 7-8, the external ankle brace 50′ may include an upright fastening system 40′ comprising at least one upright connecting strap 42′ (two shown) for selectively connecting the lateral upright extension 20′ to the medial upright extension 22′ above the ankle. It is contemplated that at least one of the upright connecting straps 42′ (or any other straps of the external ankle brace 50′) could have elastic properties to allow slight motion of the lateral and medial upright extensions 20′ and 22′ relative to one another as desired, as the external ankle brace 50′ is doffed and donned by the wearer.

Also as shown in FIGS. 7-8, the upper connecting strap 30′ is a first upper connecting strap 30′, and the upper fastening system 28′ includes a second upper connecting strap 100 for selectively connecting the lateral sidewall 18′ to the medial sidewall 16′ across the top of the shoe. The second upper connecting strap 100, when present, may be located longitudinally between the first upper connecting strap 30′ and the lateral and medial upright extensions 20′ and 22′. When rotation of the medial and lateral upright extensions 20 and 22 is “fixed” in a perpendicular position (an option discussed elsewhere herein), the second upper connecting strap 100 may be helpful to prevent plantar flexion and dorsiflexion, thus making the external ankle brace 50′ in this “non-pivoting mode” similar in overall stability to a fixed walking boot.

It is also contemplated that the first upper connecting strap 30′ (as shown in the Figures) could be omitted from the external ankle brace 50′, allowing the component shown and described herein as the second upper connecting strap 100 to serve as the (only) upper connecting strap, substantially in the position shown and described herein.

The second upper connecting strap 100 may be longitudinally wider than the first upper connecting strap 30′, as shown, for any reason. For example, in some use environments, the second upper connecting strap 100 will doing the majority of the support work (of the two upper connecting straps 100 and 30′) and be subject to the majority of the forces generated in-use. A wider strap in such circumstances distributes those forces and facilitates increased comfort for the wearer as opposed to a narrower strap (which might be more inclined to “cut in” at the edges to the wearer's foot/leg). Because the force is spread out by the wider strap, the wearer can use the external ankle brace 50′ for a long period of time without pain. The narrower first upper connecting strap 30′ is narrower, as shown, since there will not be enough room for another strap having a similar width to the second upper connecting strap 100 in many use configurations of the external ankle brace 50′.

As can be seen in at least FIG. 7, the first upper connecting strap 30′ may be permanently or adjustably attached to the respective lateral and/or medial sidewall 18′ or 16′ via a first upper fastener 102. As shown in the Figures, the first upper fastener 102 may attach the first upper connecting strap 30′ to an outer surface of the respective lateral and/or medial sidewall 18′ or 16′, such that the lateral and/or medial sidewall 18′ or 16′ is at least partially interposed between the body of the first upper connecting strap 30′ and the shoe.

Additionally, the second upper connecting strap 100, as shown in the Figures, may be permanently or adjustably attached to the respective lateral and/or medial sidewall 18′ or 16′ via a second upper fastener 104. As shown in the Figures, and in contrast to the first upper connecting strap 30′, the second upper fastener 104 attaches the second upper connecting strap 100 to an inner surface of the respective lateral and/or medial sidewall 18′ or 16′, such that the body of the second upper connecting strap 100 is at least partially interposed between the lateral and/or medial sidewall 18′ or 16′ and the shoe. In the case of the external ankle brace 50′ shown in FIGS. 7-13, this respective inner/outer fastening of the straps, at their respective positions, is considered to give a tighter and more secure fit of the external ankle brace 50′ around the wearer's shoe.

It is contemplated that one of ordinary skill in the art could similarly configure the strap placements and fastening types (number and kind of fasteners, inside/outside placement, and the like) for a particular use environment and to facilitate economical manufacture balanced with desired bracing results. For example, and as shown in FIGS. 7-8, the lower connecting strap 26′ of the external ankle brace 50′ may be located longitudinally between the first upper connecting strap 30′ and the lateral and medial upright extensions 20′ and 22′, and/or the second upper connecting strap 100 may be located longitudinally between the lower connecting strap 26′ and the lateral and medial upright extensions 20′ and 22′. As another example, in the case of the inside fastening of the second upper connecting strap 100, testing has shown that the malleability of the plastic of the external ankle brace 50′, and “lifting” of an externally-fastened upper connecting strap 100 away from the shoe by the respective lateral and/or medial sidewall 18′ or 16′, permit an undesirable degree of laxness in the fastening scheme for some use environments. That is, attaching the second upper connecting strap 100 to the inside of the lateral and medial sidewalls 18′ and 16′ may, in some use environments, assist with providing desired properties of fit, comfort, and stability. If the second upper connecting strap 100 were to be fastened outside the lateral and medial sidewalls 18′ and 16′, at the talus-adjacent position on the shoe shown in FIGS. 7-8, it may be more difficult to achieve a desired amount of comfort and stability for the wearer. As another example, in some use environments, the upper connecting strap 30′, 100 may include a variable length fastening operable for manual adjustment by a wearer to a predetermined length. As another example, the lower connecting strap 26′ may either include a variable length fastening operable for manual adjustment by a wearer to a predetermined length, or may have a constant length and not be manually adjustable by a wearer (e.g., to provide durability against a wearing surface of the shoe sole).

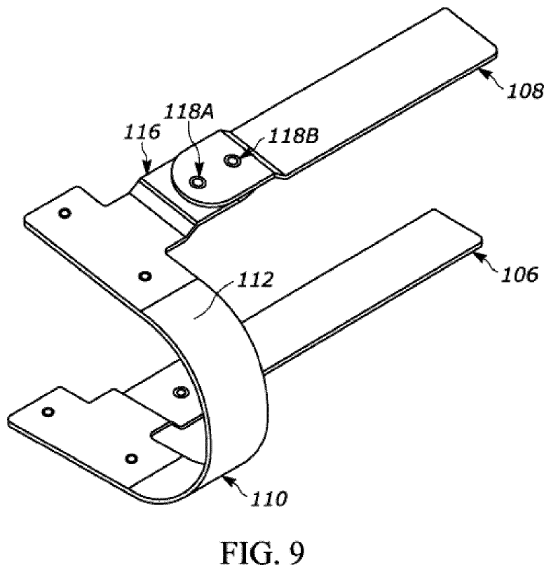

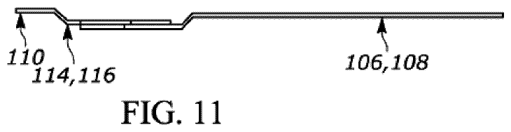

With reference now to FIGS. 8-13, the lateral upright extension 20′ may include a lateral reinforcing strut (shown in phantom view as 106 in FIG. 8), and the medial upright extension 22′ may include a medial reinforcing strut (shown as 108 in FIG. 9). The lateral and medial reinforcing struts 106 and 108, when present, may be malleable and configured to accept and maintain a nonplanar shape profile. A “nonplanar shape profile” is used herein to indicate that the component may be bent—manually and/or automatically—out of the substantially planar orientation shown for the lateral and medial reinforcing struts 106 and 108 in at least FIGS. 9 and 11. When the lateral and/or medial reinforcing struts 106 and 108 are in the nonplanar shape profile, they are configured to at least partially impart the nonplanar shape profile to the corresponding lateral or medial upright extension 20′ and 22′. In practical application, the lateral and/or medial reinforcing struts 106 and 108, and other, similar structures, can be used to help plastically and/or elastically deform the external ankle brace 50′, by a user/prescriber and/or machine, to fit the contours of a wearer's body in a desired manner. For example, if the wearer has very large or very small calf muscles, an orthotic or prosthetic assistance device, or any other feature that is different than that contemplated by a stock external ankle brace 50′, the external ankle brace 50′ can be configured (before, during, or after purchase/use) for that wearer or a like class of wearers.

The lateral and medial reinforcing struts 106 and 108 help with stability of the external ankle brace 50′ during use and may also facilitate custom fitting, or shaping the brace to the contours of an individual ankle and lower calf Without the lateral and medial reinforcing struts 106 and 108, a prescriber would likely have to heat the plastic to mold it properly, and then it may not retain its shape under wear forces during use. The lateral and medial reinforcing struts 106 and 108 assist the respective lateral and medial upright extensions 20 and 22 in being shaped easily but then also holding a shape and providing stability.

It is contemplated that the lateral and medial upright extensions 20′ and 22′ may be made of a first material, and the lateral and medial reinforcing struts 106 and 108 may be made of a second material which is more ductile than the first material. For example, the first material may be a polymer and the second material may be a metal, such as, but not limited to, aluminum. It is also contemplated that the lateral and medial reinforcing struts 106 and 108 could be made from carbon-fiber and custom-manufactured to fit a particular user or class of users.

The lateral and medial upright extensions 20′ and 22′ may wholly encapsulate the lateral and medial reinforcing struts 106 and 108. This could be accomplished, for example, by the lateral and medial upright extensions 20′ and 22′ being molded around, or otherwise fabricated to encompass, the lateral and medial reinforcing struts 106 and 108. The material of the lateral and medial upright extensions 20′ and 22′ could be significantly larger in cross-sectional size than the respective lateral and medial reinforcing struts 106 and 108, or could instead be a relatively thin “skin” (e.g., a vinyl coating) interposed between the lateral and medial reinforcing struts 106 and 108 and the ambient space. Additionally, it is contemplated that the lateral and/or medial reinforcing struts 106 and 108 could be left bare to themselves serve directly as lateral and/or medial upright extensions 20′ and 22′.

With reference now to FIGS. 8-13, the rear portion 12′ may include a rear reinforcing strut 110. The rear reinforcing strut 110 may be malleable and configured to accept and maintain a nonplanar shape profile, to at least partially impart the nonplanar shape profile to the rear portion 12′, although it is contemplated that the rear reinforcing strut 110 could also or instead be relatively rigid and configured to maintain the lateral and medial reinforcing struts 106 and 108 in a predetermined spacing and mutual orientation relationship (e.g., to avoid “torquing” or “twisting” of the lateral and medial upright extensions 20′ and 22′. despite any reconfiguring of the lateral and/or medial reinforcing struts 106 and 108).

As with the external ankle brace 50 of FIGS. 1-6, the rear portion 12′ of the external ankle brace 50′ of FIGS. 7-13 encircles the heel, but leaves the heel of the shoe exposed so that the user's shoe can directly contact the ground. The rear portion 12′ of the rigid heel enclosure 10′ is cut out to leave the bottom of the heel, and a portion of the side of the heel, of the shoe exposed. As depicted in at least FIG. 7, the back of the external ankle brace 50′ may fit or rest on the base of the heel counter of the shoe, which is not the base of the shoe. The external ankle brace 50′ does not touch the ground at the heel of the shoe and there is a gap (e.g., about a half inch, for some users) between the sole of the shoe and the rigid heel enclosure 10′.

Whether or not deformation of the rear reinforcing strut 110 is contemplated, the rear portion 12′ may be made of a first material and the rear reinforcing strut 110 may be made of a second material which is more ductile than the first material. For example, the first material may be a polymer and the second material may be a metal. For example, the first material may be a polymer and the second material may be a metal, such as, but not limited to, aluminum. It is also contemplated that the rear reinforcing strut 110 could be made from carbon-fiber and custom-manufactured to fit a particular user or class of users.

The rear portion 12′ may wholly encapsulate the rear reinforcing strut 110. The rear portion 12′ may be molded around the rear reinforcing strut 110. The material of the rear portion 12′ could be significantly larger in cross-sectional size than the rear reinforcing strut 110, or could instead be a relatively thin “skin” (e.g., a vinyl coating) interposed between the rear reinforcing strut 110 and the ambient space. Additionally, it is contemplated that the rear reinforcing strut 110 could be left bare to itself directly serve as a rear portion 12′. A material selection (e.g., a high-friction material) and/or surface treatment (e.g., knurling) may be used on the rear portion 12′ (or directly on the rear reinforcing strut 110, when serving as the rear portion 12′) to increase frictional surface area contacting the heel counter of the shoe to provide desired fixation to help restrict plantar flexion and dorsiflexion. (As with several components of the external ankle brace 50′, including, but not limited to, the lateral and medial upright extensions 20′ and 22′ and the rear and forward portions 12′ and 14′, it may be desirable to balance manufacturing considerations, friction of surfaces, area of surfaces, and weight of components in seeking comfort, stability, security/tightness, motion restriction, and usability for the wearer.)

The rear reinforcing strut 110 may include a curved rear strut body 112 extending around the heel portion of the shoe and lateral and medial strut stubs 114 and 116, respectively, extending substantially perpendicularly from the rear strut body 112, at opposed locations on the rear strut body 112. The lateral and medial reinforcing struts 106 and 108 are directly pivotally connected to the lateral and medial strut stubs 114 and 116, respectively. The lateral, medial, and/or rear reinforcing struts 106, 108, and 110, when present, may provide desired rigidity and/or strength, such as to permit a lower-profile size, to the respective lateral and medial upright extensions 20′ and 22′ and/or rear portion 12′.

As shown in the Figures, the rear reinforcing strut 110 may be directly pivotally connected to the lateral and medial reinforcing struts 106 and 108 in any desired manner. For example, at least one restraining bolt 118 may be connected to a chosen one of the lateral and medial upright extensions 20′ and 22′ and be operative to selectively restrict pivoting of the chosen upright extension 20′ and 22′ respective to a corresponding lateral or medial sidewall 18′ or 16′. In FIG. 7, this configuration is shown as a primary restraining bolt 118A and a plurality (three shown) of secondary restraining bolts 118B. The primary restraining bolt 118A in FIG. 7 is located at a pivot point of the lateral ankle joint 32′. The secondary restraining bolts 118B, when present, resist pivoting of the respective lateral or medial upright extension 20′ and 22′ with respect to a corresponding lateral or medial sidewall 18′ or 16′. Thus, restraining bolts 118A and/or 118B may be connected to a chosen one of the lateral and medial reinforcing struts 106 and 108 and may be operative to either assist/facilitate or selectively restrict pivoting of the chosen upright extension 20′ and 22′ respective to the rear reinforcing strut 112.

As another example, FIGS. 8-13 depict a configuration of the external ankle brace 50′ shown as having one restraining bolt 118A at a center pivot location of the lateral or medial ankle joint 32 and 34, and at least one second restraining bolt 118B at a location off-center from the pivoting point but still configured to selectively attach the respective lateral or medial upright extension 20′ or 22′ directly to a corresponding structure associated with the ankle joint 32 or 34 and thus substantially prevent relative pivoting therebetween. For example, and as shown in at least FIGS. 9-12, the second restraining bolt 118B may selectively mutually connect the lateral or medial strut stub 114 or 116 to a respective lateral or medial reinforcing strut 106 or 108.

It is contemplated, however, that one or more second restraining bolts 118B could instead be used at a center pivot location of the lateral or medial ankle joint 32 and 34, and at least one restraining bolt 118A could be placed at a location off-center from the pivoting point but still configured to selectively attach the respective lateral or medial upright extension 20′ or 22′ directly to a corresponding structure associated with the ankle joint 32 or 34 and thus substantially prevent relative pivoting therebetween as desired. In this alternate situation, the second restraining bolt 118B shown in FIG. 8 could be located for pivotal alignment with the malleolus and the first restraining bolt 118A could be off-center for selectively preventing pivoting. That is, the off-center restraining bolt(s) 118—regardless of specific configuration—could be manipulated by a prescriber or user to prevent pivoting of one or both of the lateral or medial upright extensions 20′ or 22′ with respect to the respective lateral or medial sidewalls 18 and 16 at a desired “restrict second direction pivoting” time, and could be manipulated to allow at least partial pivoting of one or both of the lateral or medial upright extensions 20′ or 22′ with respect to the respective lateral or medial sidewalls 18 and 16 at a desired “allow second direction pivoting” time. One of ordinary skill in the art will be able to provide one or more restraining bolts 118 having a desired type, location, and other physical configuration for a particular use environment of the external ankle brace 50′.

Regardless of how the restraining bolts 118 are configured and located on the various other components of the external ankle brace, it is contemplated that the restraining bolts 118 could be manipulated by any suitable party, at any desired time before, during, and/or after wear of the external ankle brace 50 and 50′, and for any desired reason. For example, the pivoting could be further restricted once an already tender ankle is further stressed, or the pivoting could be further permitted if a previously tender ankle responds well to light, restricted-pivoting duty. It is contemplated that one or more components of the external ankle brace 50 and 50′ could include a slot (e.g., a curved slot) within which a corresponding restraining bolt 118 can relatively travel or slide during use, in order to permit a limited amount of pivoting. It is also contemplated that one or more of the restraining bolt(s) could be a Chicago screw/bolt type, include a post and/or sleeve feature, or otherwise be configured to facilitate smooth rotation (and/or avoid wear) between two or more components of the external ankle brace 50 and 50′, whether or not they are permitted to selectively pivot relative to one another.

Via the aspects of the external ankle braces 50 and 50′ shown and described herein, a user can place the external ankle brace 50 and 50′ around an existing shoe (thus obviating the expense and inconvenience of special and/or mismatched shoes to accommodate an inside-the-shoe brace), tighten as desired, and accordingly selectively restrict movement of an ankle in a first direction and selectively permit movement of the ankle in a second direction (e.g., through selective use of the pivoting restriction schemes described above). The external ankle brace 50 also restricts rotation of the ankle (“windshield wiper” motion of the toes with a stationary heel, or vice versa). Accordingly, an ankle can receive a desired amount of support—capable of changing very quickly, even during a single wear session (e.g., an athletic or daily-activity event) via use of the restraining bolt(s) 118—and thus avoid initially or additionally injuring an ankle, foot, leg, or other portion of the wearer's body.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Padding or other cushioning material could be placed on any desired surface(s) of the components of the external ankle brace 50, 50′ to assist with force absorption, spacing, abrasion resistance, or for any other reason. A restraining bolt 118 could be selectively associated with only a chosen one of the lateral and medial upright extensions 20′ and 22′ to prevent with respect to the respective lateral and medial sidewall 18 and 16 at a desired “restrict second direction pivoting” time, with pivoting of the other of the lateral and medial upright extensions 20′ and 22′ being indirectly limited via the connection to the other of the lateral and medial upright extensions 20′ and 22′ through the upright connecting strap(s) 42. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Embodiments include an external gauntlet ankle/foot orthosis which applies over the shoe providing ankle and foot stability prophylactically or following acute or chronic trauma. Embodiments relate to an orthotic that limits or prevents ankle inversion, eversion, dorsiflexion and plantar flexion and well as providing mid foot support.

Embodiments can minimize ankle inversion and eversion during physical activity and/or minimize ankle medial and later rotation during physical activity and/or minimize ankle plantar flexion and dorsiflexion during physical activity and/or provide stability to the mid foot in limiting pronation and supination of the foot.

Embodiments can include a foot/ankle orthotic that includes a lateral sidewall, a medial sidewall, a heel enclosed backing connecting the sidewalls, a lateral upright extension, a medial upright extension and a bottom strapping system connecting sidewalls. The lateral and medial upright extensions are attached to the sidewalls with an overlapping ankle joint off-set to accommodate for medial and lateral malleolus anatomical positioning. The lateral sidewall coincides with the outer or exterior portion of the foot/ankle and the medial sidewall coincides with the inner portion of the foot/ankle. The lateral upright extension coincides with the outer or exterior portion of the lower leg and the medial upright extension coincides with the inner portion of the lower leg. Lateral and medial extension walls are configured to rise above the ankle of the wearer of the orthotic by approximately 8-9 inches (from the bottom of the hinge to the top of the extension walls). When donned by the wearer, lateral and medial side walls also partially wrap over the top or dorsum of the foot leaving a gap of approximately 3 to 4 inches between the sidewalls. The width of the medial and lateral upright extensions is approximately 3-4 inches wide.

A feature of an embodiment is to have the securing mechanism include a hook and loop strap across the dorsal (top) of the foot. This Velcro securing strap is riveted to the in place on both the medial and later side walls. A D ring is utilized on the lateral fixation in which the Velcro strap can be fed through and secured back upon itself. The lateral and medial upright extensions are secured by two removable Velcro straps and D rings. Male component Velcro is adhesively attached to each upright and the female component Velcro strap can connect to the uprights are desired positions for appropriate fitting. As an option, the brace may also be applied with various types of athletic adhesive tape in conjunction with or instead of the Velcro strapping and D ring system.

Another feature of an embodiment is an overlapping ankle joint hinge to allow the ankle to move freely through plantar flexion and dorsiflexion. The overlapping ankle joint is located on the medial and lateral aspects of the gauntlet where the medial and lateral side bodies attach with the medial and lateral uprights respectively. The ankle joint hinge components are off set to produce a more anatomically correct gauntlet for a more fluid mobility.

Foam padding (approximately ¼ inch) is attached to the inside of both the medial and lateral uprights to provide additional comfort and protection for the wearer. The gauntlet is sized so that one size can fit multiple size shoes. A separate gauntlet is needed to accommodate both right and left ankles.

A sheet of vacuum formable thermoplastic large enough to cover the entire mold is cut and placed in an oven to be heated to a formable temperature. These are several types and thicknesses of plastic that may be used for this fabrication including orthotic grade polypropylene, polyethylene, and copolymer.

Embodiments include the combination of any one or more elements/features disclosed herein with any one or more other elements/features disclosed herein, unless otherwise specified, providing that the art enables such. Embodiments also include the combination of any one or more elements/features of any of Ser. No. 15/074,339, filed 18 Mar. 2016, U.S. patent application Ser. No. 15/642,430, filed Jul. 5, 2017, U.S. patent application Ser. No. 16/374,865, and/or Provisional Patent Application Ser. No. 62/135,823, filed 20 Mar. 2015, with any one or more other elements/features disclosed herein, including one or more of the elements/features in any one or more of the Appendices and/or with any one or more other elements/features of the above noted patent applications, unless otherwise specified, providing that the art enables such. Embodiments also include the exclusion of any one or more elements/features disclosed herein with any one or more other elements/features disclosed herein, unless otherwise specified, providing that the art enables such. Embodiments also include the exclusion of any one or more elements/features of any of Ser. No. 15/074,339, filed 18 Mar. 2016, U.S. patent application Ser. No. 15/642,430, filed Jul. 6, 2017, U.S. patent application Ser. No. 16/374,865, and/or Provisional Patent Application Ser. No. 62/135,823, filed 20 Mar. 2015, with any one or more other elements/features disclosed herein, including one or more of the elements/features in any one or more of the Appendices and/or with any one or more other elements/features of the above noted patent applications, unless otherwise specified, providing that the art enables such. Unless defined otherwise, all technical and scientific terms used herein have the same meaning as is commonly understood by one of skill in the art to which the present disclosure pertains.

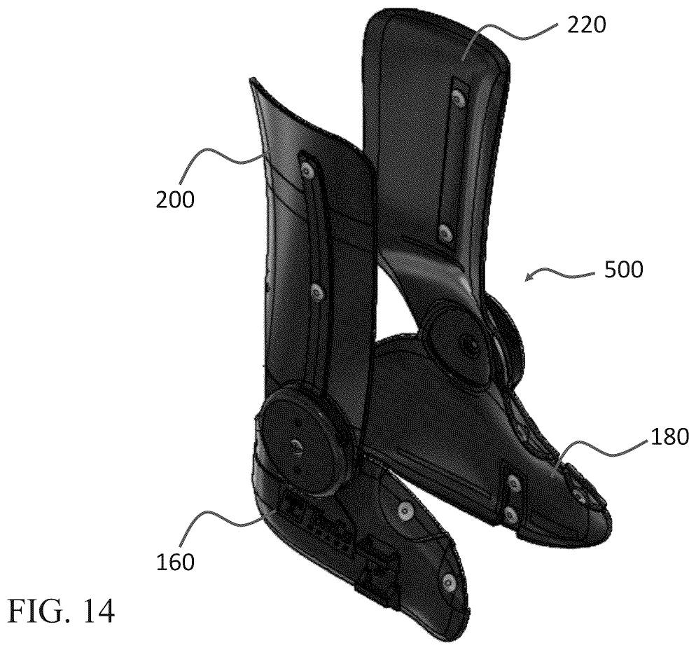

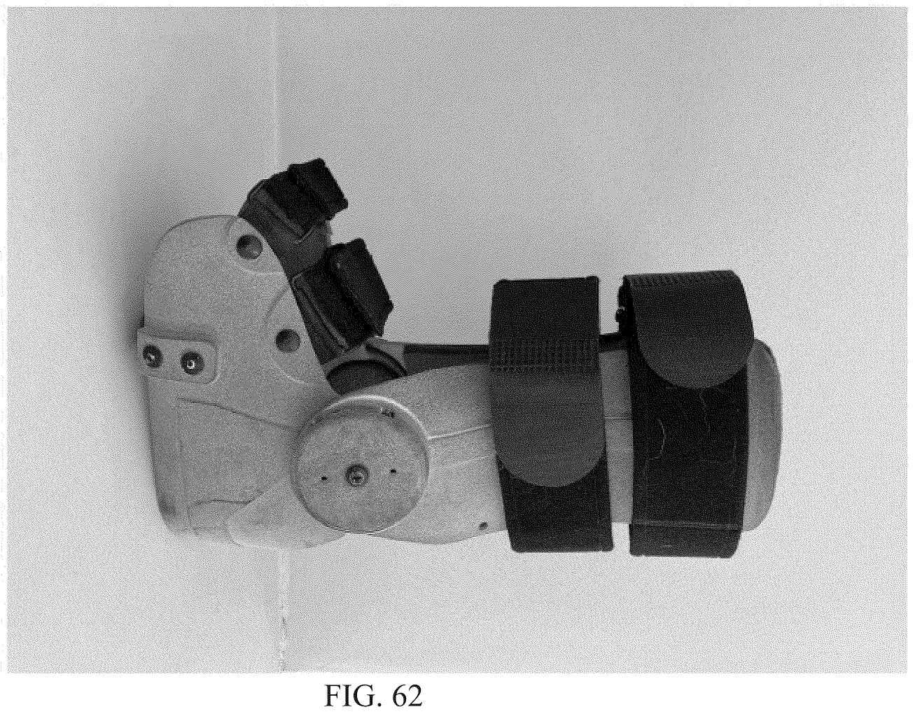

FIGS. 14 and 62 present another, different embodiment, from the embodiments of FIGS. 1-13 described above. By way of example only and not by way of limitation, the embodiments of the external ankle brace 500 of FIG. 14 and onward include an adjustable bottom strap and a three or four or more state adjustable hinge. Moreover, embodiments of the external ankle brace 500 of FIG. 14 and up for the most part include a medial upright extension that is anatomically correct. For example, the upright is contoured to accommodate the medial malleolus. These aspects another aspect will be described in greater detail below. Unless otherwise noted, all of the disclosure from here forward corresponds to a disclosure relating to embodiments that are different from the embodiments of FIGS. 1 to 13. That said, the embodiments disclosed below can use one or more of the features of the embodiments of FIGS. 1 to 13 described above. Indeed, the following description below will not address at least some of the features that are common with the embodiments above (e.g., the upper fastening system, the strap used with the uprights, etc.), but at least some of those features are also present in the embodiments described below. Any interpretation of claim language relating to 35 USC 112, sixth paragraph/(f) corresponds to the embodiments detailed below, although such claims can also include the features detailed above if there is no feature disclosed below that falls within such claim construction. And note that embodiments can include combining one or more of the features below with one or more of the features of the aforementioned patent applications unless otherwise noted providing the art enables such (thus this constitutes additional disclosure made by way of reference to achieve textual economy).

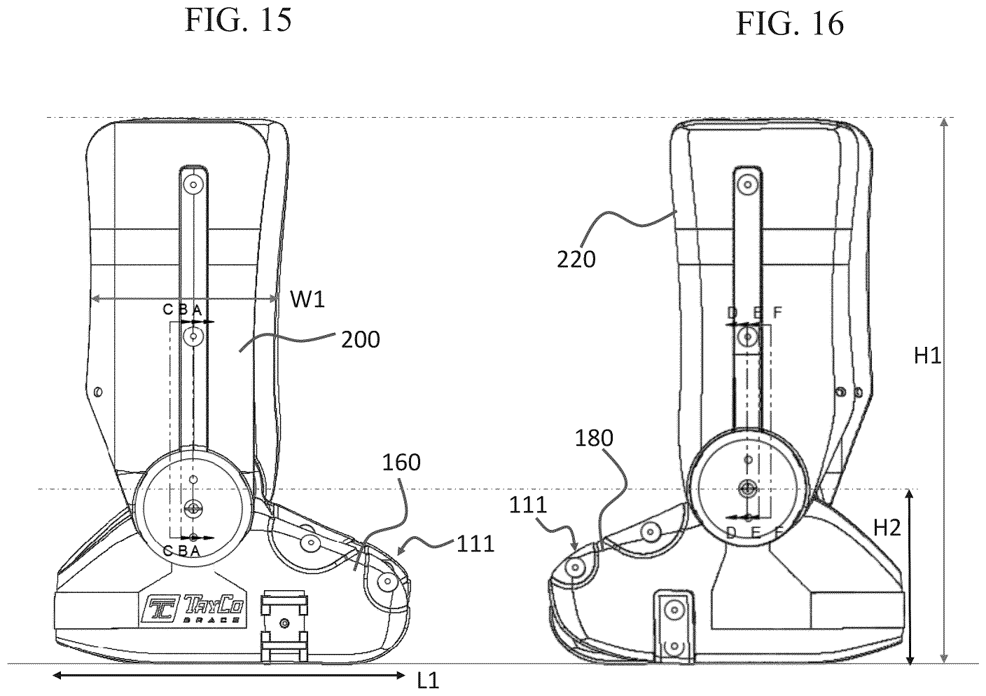

FIGS. 15 and 16 presents side views of the extra level brace 500 according to an exemplary embodiment. Here, it can be seen that there are lateral upright extension 200 and medial upright extension 220. In an exemplary embodiment, these uprights can have one or more of the features detailed above with respect to the uprights of the embodiments of FIGS. 1 to 13. These uprights are jointly connected to the rigid heel enclosure 96. The rigid heel and closure 96 includes a lateral sidewall 160, and a medial sidewall 180. Again, the sidewalls can have one or more the features detailed above with respect to the sidewalls of the embodiments of FIGS. 1 to 13. As will be described in greater detail below, the joints of the respective lateral and medial upright extensions have a male-female joint assembly, some respect to portions of which are monolithic with the remainder of the bulk of the extensions and sidewalls respectively. Again, additional details of this will be described below.

FIG. 16 shows a dimension H1, which is the height from the bottom to the top of the brace 500. In an exemplary embedment, H1 can be less than and/or equal to and/or greater than (meaning that it can be less than one of the following numbers and greater than another of the following numbers for example) 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75 or 15 inches or any value or range of values therebetween in 0.0001 inch increments (to achieve English units for example). And note that the sides of the brace can be different heights (e.g., the top of the lateral upright extension can be at a different height than the top of the medial upright extension), and thus one side can have one of the aforementioned values, and the other side can have another of those values. Indeed, embodiments are such that that is the case. In an exemplary embodiment, H1 on one side, such as the medial side, can be less than, equal to and/or greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18% or more or any value or range of values therebetween in 0.01% increments than the H1 on the opposite side (where the smaller one is the denominator).

FIG. 15 shows a dimension Wi, which is the width of the upright 200 (and can be a width of the upright 220) at its narrowest portion in the parabolic portion. In an exemplary embodiment, H1 can be less than and/or equal to and/or greater than 2.5, 2.75, 3, 3.25, 3.5, 3.75, 4, 4.25, 4.5, 4.75, 5, 5.25, or 5.5 inches or any value or range of values therebetween in 0.0001 inch increments (to achieve English units for example). And note that these drawings herein (all drawings unless otherwise noted) are to scale, and thus disclosures constitute values that are scaled relative to other specific values for purposes of textual economy.

FIG. 16 shows a dimension H2, which is the height of the axis of rotation of the medial joint/hinge from the bottom of the medial sidewall. In an exemplary embodiment, H2 can be less than and/or equal to and/or greater than 2, 2.25, 2.5, 2.75, 3, 3.25, 3.5, 3.75, 4, 4.25, 4.5, 4.75 or 5 inches or any value or range of values therebetween in 0.0001 inch increments (to achieve English units for example).

FIG. 15 shows a dimension L1, which is the length of the lateral sidewall (and can be the length of the medial sidewall) from the rearmost end to the foremost end. In an exemplary embodiment, L1 can be less than and/or equal to and/or greater than 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75 or 11 inches or any value or range of values therebetween in 0.0001 inch increments (to achieve English units for example).

It is noted that the dimensions disclosed herein and the values disclosed herein are values and dimensions associated with the brace in a completely relaxed state unless otherwise noted. In this regard, tensioning of the straps and/or the action of wearing the brace can result in deformation of the structures of the brace, even though those structures are rigid (more on this below). Accordingly, the dimensions and values disclosed herein can be dimensions that corresponded components in a completely disassembled state and/or components in a completely relaxed state and/or a state where all straps are completely removed. In an exemplary embodiment, the dimensions and values disclosed herein are those that exist when the brace is positioned in exact alignment of the X, Y and Z axes. In an exemplary embodiment, the dimensions and values disclosed herein are those that correspond to the dimensions and values when the lower connecting strap is completely removed and in no way attached to the sidewalls and the brace is positioned so that the bottoms of the sidewalls are located on a flat level surface without any other forces of the gravity acting on the brace.

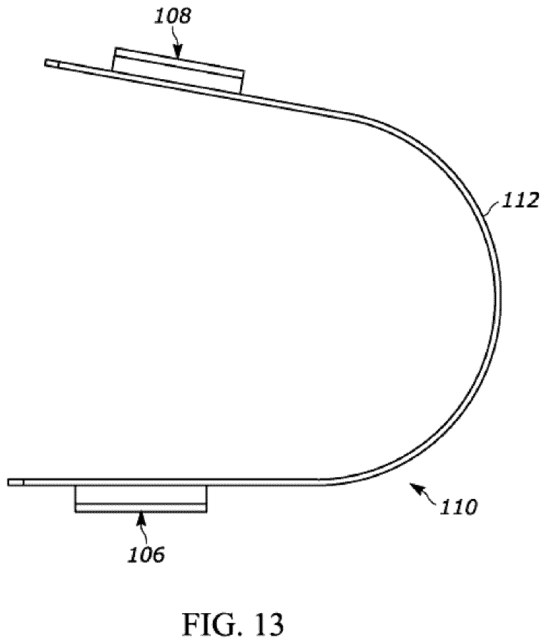

These dimensions are given so that the other portions of the brace can be scaled to within 10, 9, 8, 7, 6, 5, 4, 3, 2 or 1%. Herein, the figures of FIG. 13 and thereafter are to scale (more on this below).

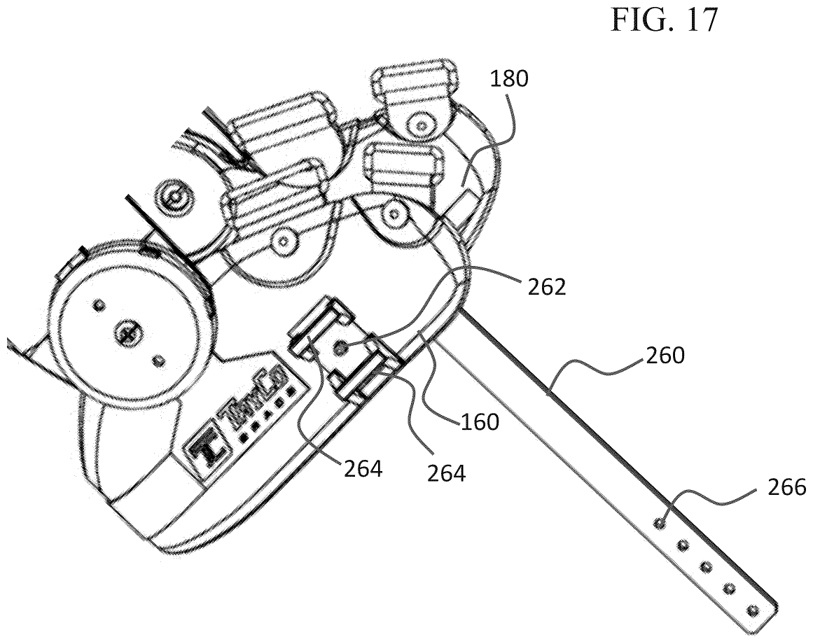

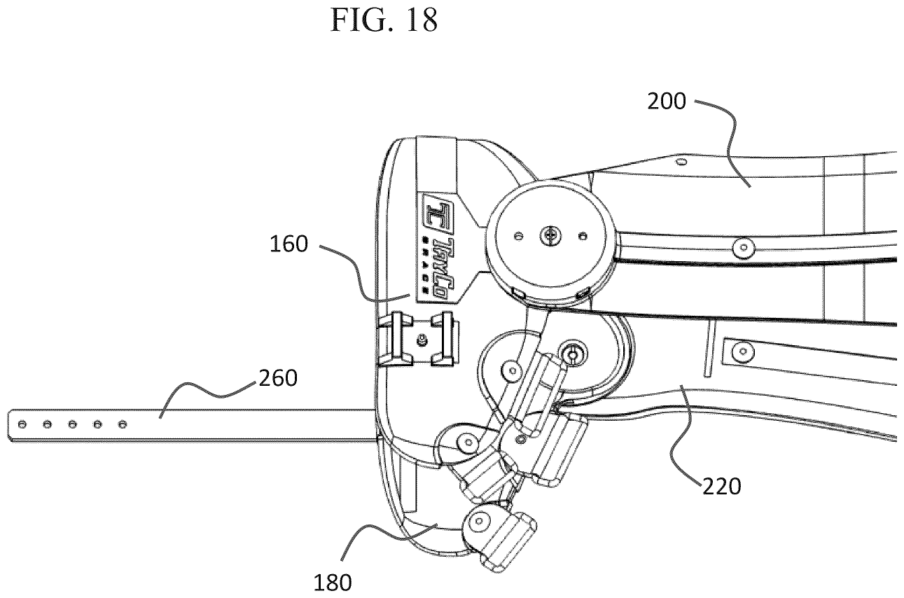

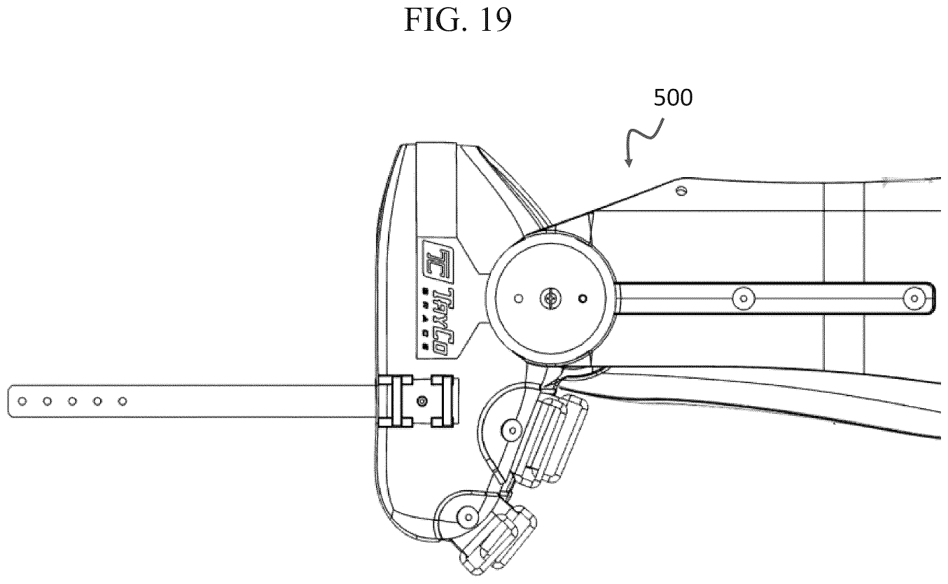

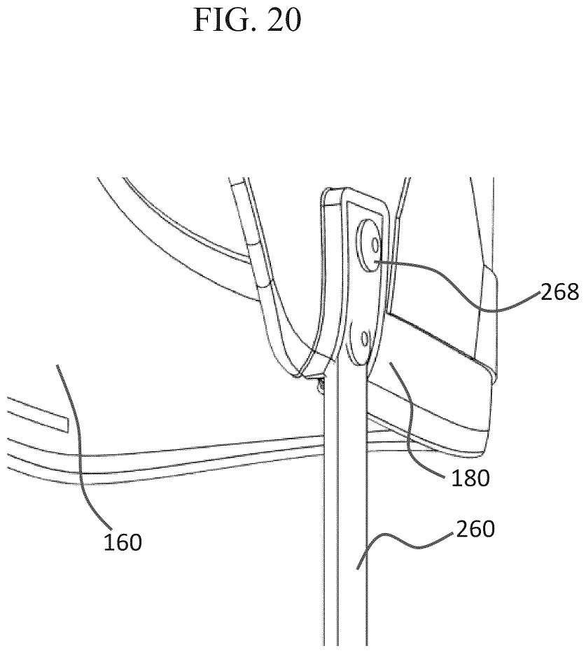

FIGS. 18 to 22 provide additional views of the external ankle brace 500 according to exemplary embodiments. FIGS. 18 to 20 show the lower connecting strap 260. Unlike the embodiments of FIGS. 1 to 13, the lower connecting strap in this embodiment is configured to be adjustable and is configured to have one end that can be freed by a user from connection with the respective sidewall. In these embodiments, it is the lateral sidewall where the releasable connection is present, although in other embodiments, it can be the medial sidewall and/or can be both sidewalls in some embodiments. Still, with respect to the embodiment shown in the figures, the lower connecting strap 260 is fixedly mounted to the medial sidewall 180 by 2 rivets 268. One rivet can be used, or three or more rivets can be used. Bolts or Chicago screws can be utilized. In an exemplary embodiment, the medial sidewall can be molded around the strap 260. By way of example only and not by way limitation, holes through the strap 260 can be present, and during the molding operation, the material of the sidewalls will flow into the holes and thus create a positive retention between the strap and the sidewall. That said, alternatively and/or in addition to this, a male portion at the end of the strap can be present such that the sidewalls will be molded around the strap thus also creating positive retention (like molding around the top of a T or the bottom of an L—the character cannot be pulled own or up, respectively, because of the material around the horizontal portions.

In an exemplary embodiment, the strap 260 can have a width (in the long direction of the rigid heel enclosure) of less than and/or equal to and/or greater than 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9 or 2 inches or more or any value or range of values therebetween in 0.0001 increments (or less), at least to achieve the standard English measurements (e.g., 0.375 inches, 0.625 inches, 0.0625 to 0.875 inches, etc.). The strap 260 can have a thickness of less than and/or equal to and/or greater than 0.03, 0.04, 0.05, 0.06, 0.07, 0.08, 0.09, 0.1, 0.125, 0.15, 0.12, 0.13, 0.14, 0.15, 0.16, 0.17, 0.18, 0.19, 0.20, 0.21, 0.22, 0.23, 0.24, 0.25, 0.3, 0.35 inches or more or any value or range of values therebetween in 0.0001 increments (or less), at least to achieve the standard English measurements (e.g., 0.125 inches, 0.0625 inches, 0.0625 to 0.33 inches, etc.).

The length can be less than and/or equal to and/or greater than 3, 3.5, 4, 4.5, 5, 5.5, 6, 6.5, 7, 7.5, 8, 8.5, 9, 9.5, 10, 10.5, 11, 11.5, 12, 12.5 or 13 inches or more or any value or range of values therebetween in 0.0001 increments (or less), at least to achieve the standard English measurements. In an exemplary embodiment less than and/or equal to and/or greater than 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2, 2.25, 2.5, 2.75, 3, 3.25 or 3.5 inches or more or any value or range of values therebetween in 0.0001 increments (or less), at least to achieve the standard English measurements (e.g., 0.375 inches, 0.625 inches, 0.0625 to 0.875 inches, etc.) on the strap 260 can be overlapping and/or embedded in the sidewall to which the strap is fixedly attached.

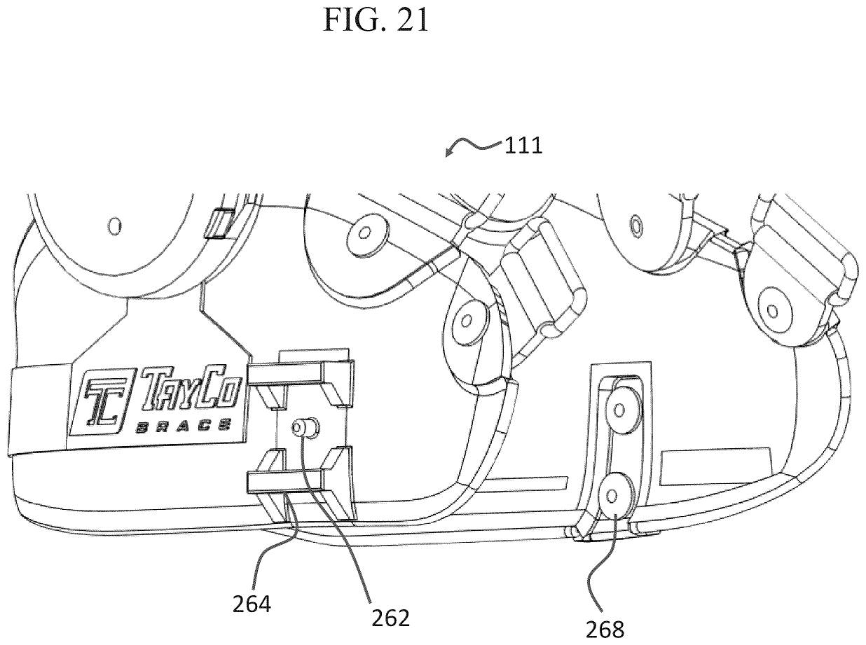

FIG. 21 shows a view of the rigid heel enclosure 111 without the strap 260 for clarity. This shows the inside of the medial sidewall, and showing the interior facing heads of the rivets 268 that are utilized to secure the strap to the sidewall. Also shown is the cavity in the sidewall on the interior side thereof that is sized and dimensioned to accept the end of the strap 260. In an exemplary embodiment, the cavity is sized and configured so that the strap is at least flush with the surface on either side of the cavity so that the strap does not stand proud of the surface, and thus create a pressure point against the shoe. In an exemplary embodiment, the strap and/or the cavity is sized and dimensioned so that the heads of the minutes 268 are also not proud or otherwise are flush with the surrounding surface of the sidewall. In an exemplary embodiment, the surrounding surface is proud of the side of the strap and/or the highest most portion of the rivets 268 (and thus in the direction facing the shoe when the braces worn) by zero inches (flush) and or less than and/or equal to and/or greater than 0.03, 0.04, 0.05, 0.06, 0.07, 0.08, 0.09, 0.1, 0.125, 0.15, 0.12, 0.13, 0.14, 0.15, 0.16, 0.17, 0.18, 0.19, or 0.20 inches or more or any value or range of values therebetween in 0.0001 increments (or less), at least to achieve the standard English measurements.

Unless otherwise noted, all dimensions and or measurements disclosed herein are in inches (below metric values are used).

FIG. 21 also shows the fixed buckle portion of the lower connecting system that enables the releasable connection of the strap 260 to the lateral sidewall. In this exemplary embodiment, there are two female portions 264 and a single male portion 262.

In use, the free end of the strap 260 is snaked through the lower female receptacle 264, and then over the male portion 262, and then into the upper female receptacle 264, and then a desire tightness is achieved by pulling and/or pushing the strap 260 further through at least one of the receptacles in combination with the alignment of one of the holes 266 in the strap 260 aligning over the top of the male portion 262. The strap is then pressed towards the lateral sidewall so that the male portion 62 extends into the desired hole 266 in the strap 260 to connect the strap 260 to the lateral sidewall in a manner that will keep the strap from pulling out of the female receptacles by at least 0.01, 0.02, 0.03, 0.04, 0.05, 0.06, 0.07, 0.08, 0.09, 0.1, 0.125, 0.15, 0.12, 0.13, 0.14, 0.15, 0.16, 0.17, 0.18, 0.19, 0.20, 0.21, 0.22, 0.23, 0.24, or 0.25 inches or any value or range of values therebetween in 0.0001 increments (or less), at least to achieve the standard English measurements.

In an exemplary embodiment, the strap and/or the structure that supports the strap (e.g., the sidewalls and/or the male portion 262, for example), are configured to withstand a tension of at least and/or equal to and/or no more than 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 pounds any value or range of values therebetween in 0.01 pound increments without experiencing a failure mode. In an exemplary embodiment, the aforementioned tensions can be applied without experiencing an effective deleterious degradation in the strap and/or the support structure. And to be clear, the aforementioned values need not the same for both the strap and the support structure. They can be different. In an exemplary embodiment, the strap can be configured to fail before the support structure/connecting structure or vice versa, for a given tension.

The desire tautness and/or limitation of pullout can be achieved in part based on the material properties of the strap and/or the size and or dimensions of the strap. In an exemplary embodiment, the strap is made of a polymer that is both flexible and resistant to wear. In an exemplary embodiment, the strap 260 extends beneath the sole of the shoe and thus interfaces with the ground upon which the user walks and/or runs. In an exemplary embodiment, the strap when utilized with the bottom of a shoe is such that when a standard sneaker and/or a standard shoe is worn, a portion of the sole of the shoe does not contact the ground even though that portion is on a side otherwise spaced away from the strap. That is, the strap supports a portion of the soul of the shoe, and depending on the flexibility of the soul of the shoe, there will be a portion that is held or otherwise supported away from the ground until the sole of the shoe flexes towards contact with the ground.

In an exemplary embodiment, the strap 260 is sized and dimensioned so that a bending radius of less than and/or equal to and/or greater than 0.01, 0.02, 0.03, 0.04, 0.05, 0.06, 0.07, 0.08, 0.09, 0.1, 0.125, 0.15, 0.12, 0.13, 0.14, 0.15, 0.16, 0.17, 0.18, 0.19, 0.20, 0.21, 0.22, 0.23, 0.24, or 0.25 inches or any value or range of values therebetween in 0.0001 increments (or less), at least to achieve the standard English measurements, can be achieved 30, 35, 40, 45, 50, 55, 60, 65, 70, 75, 80, 85, 90, 95, 100, 125, 150, 175, 200, 225, 250, 275, 300, 325, 350, 375, 400, 450, 500, 550, 600, 700, 800, 900 or 1000 times or more or any value or range of values therebetween in 1 increment from a completely straight state without stretching and/or without reducing the strength of the strap by an effectively deleterious amount.

In an exemplary embodiment, the aforementioned performance features of the strap and or connecting structure are such that no elastic deformation of the pertinent component occurs. In an exemplary embodiment, the aforementioned performance features of the strap and/or connecting structure are such that to the extent plastic deformation occurs, the plastic deformation is limited by an amount that still enables the external ankle brace to be an effective external ankle brace or otherwise to maintain efficacy for at least 30, 35, 40, 45, 50, 55, 60, 65, 70, 75, 80, 85, 90, 95, 100, 125, 150, 175, 200, 225, 250, 275, 300, 325, 350, 375, 400, 450, 500, 550, 600, 700, 800, 900 or 1000, 1250, 1500, 1750, 2000, 2500, 3000 or any value or range of values therebetween in one increment hours of use and/or usages of the brace (one usage is putting the brace on and taking it off, with potentially some walking/running in between).

In an exemplary embodiment, the aforementioned performance features of the strap and/or the connecting structure are such that all things being equal, when the strap and the connecting structure are tested with a standardized and/or model shoe of male size 9 in US measurements that has a rigidity at least 10 times higher than that of the rigid heel enclosure (e.g., a model shoe made out of wood or iron or aluminum), an elastic extension of the strap from a distance from the location where the strap “leaves” contact with the sidewall to the location where the strap comes into contact with the opposite sidewall does not expand more than and/or equal to 0.1, 0.2, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2, 2.25, 2.5, 2.75, 3, 3.25, 3.5, 3.75, 4, 4.5, 5, 5.5, 6, 6.5, 7, 7.5, 8, 8.5, 9, 9.5, or 10 percent or any value or range of values therebetween in 0.01 increments (or less), relative to the strap when new and not having undergone any of the performance features or having undergone any of the performance features 1, 2, 3, 4, 5, 6, 7, 8, 9 and/or 10 times and/or hours.

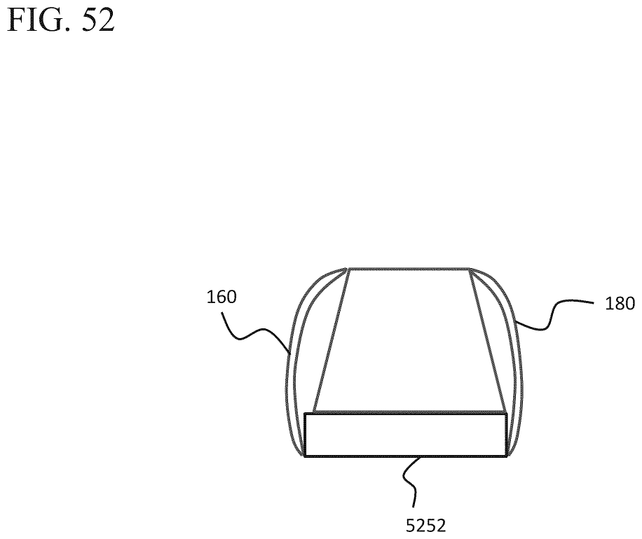

FIG. 52 shows an exemplary standard block arrangement 5252 that can be used to test the aforementioned performance features. This simple block arrangement can be utilized in various dimensions providing that it is sufficiently structurally dimensionally stable and otherwise will not flex or give in a meaningful amount upon the tensioning of the upper strap and/or the lower strap. The block can be positioned as shown (where the minimum amount of extension past the bottom lips is used (so as to not interfere with the bottom strap) and/or in any other position that does not interfere with the bottom strap.

The utilization of an adjustable lower connecting strap 260 enables the distance between the sidewalls at the top and/or at the bottom to be varied for the same size shoe and/or foot and/or control standard. Briefly, it is noted that the bottom strap has 2, 3, 4, 5, 6, 7, 8, 9 and/or 10 holes or any value or range of values therebetween in 1 hole increments every half inch along the length of the strap, in other embodiments, the holes can be present every quarter inch or every two thirds of an inch or every three eights of an inch, etc. The holes need not necessarily be evenly distributed along the length of the strap. The distance between one hole from another hole can be less than or equal to and/or greater than 0.1, 0.2, 0.3, 0.4, 0.5, 0.6, 0.7, 0.8, 0.9 or 1 inch or any value or range of values therebetween in 0.0001 inch increments.

And it is briefly noted that while a traditional belt buckle concept has been proffered in the embodiment shown, in other embodiments, other types of connection regimes can be utilized, such as a Velcro arrangement (which would permit adjustment in quasi-infinitely incremental amounts) and/or a tile can be utilized, etc.

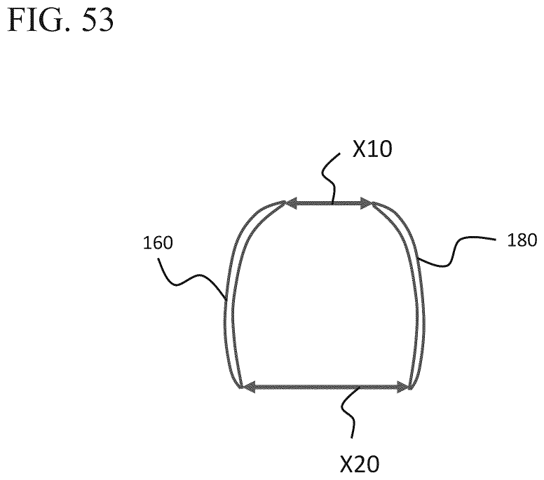

But in any event, it is noted that the adjustable lower strap can enable the distance between the sidewalls of the top and/or at the bottom to be varied for the same size shoe and/or foot and/or control standard. With reference to FIG. 53, it can be seen that there are two dimensions shown, X10 and X20. X10 is the distance between the top of the sidewalls at locations on a plane that is parallel to and on the direction of looping of the strap 260, and X20 is the distance between the bottom of the sidewalls at locations on a plane that is parallel to and on the direction of looping of the strap 260. That is, the measurements are taken at the strap. It is noted that in an exemplary embodiment, the plane can be located immediately forward and/or immediately and back of the strap, to account for embodiments where the sidewall has a cavity for the strap at the location where there is permanent attachment. In any event, the distance X20 can be adjusted, for example, by the increments of the holes in the strap, to be a value less than and/or equal to and/or greater than 1.5, 2, 2.5, 3, 3.5, 4, 4.5, 5, 5.5 or 6 inches or any value or range of is therebetween in 0.0001 inch increments, and the distance X10 is also adjustable, by increments of the upper strap, for example, which could be an infinite adjustment gradient where the strap utilizes Velcro, to be a value less than and/or equal to and/or greater than 1, 1.5, 2, 2.5, 3, 3.5, 4, 4.5, 5, or 5.5 inches or any value or range of is therebetween in 0.0001 inch increments, all depending on the standard that is utilized, whether that be a shoe or a block or test article, etc. The point is, unlike a fixed lower strap, where the distance X10 would often be an effectively “fixed” distance because it would be governed by the overall shape of the shoe or standard, where the bottom distance would also be a fixed distance because the bottom strap is fixed, by enabling adjustments of the bottom strap, this renders the distance X10 to be a variable distance for the same size shoe and/or foot and/or standard. That is, if let us say for example X20 would be a distance of 4 inches for a shoe that has a given size, the distance X10 for that shoe would always be about the same when the sidewalls were tightened around the shoe by the same amount of tautness (again, this is all things being equal). However, if the distance X20 was adjustable to 3.75 inches, the distance X10 would be different for the same tautness for the same size shoe or standard. Embodiments thus include ratios of X10 to X20 to achieve the same tautness for the same size shoe or standard. That is, the ratios of X10 over X20 can be different for the same exact shoe and/or foot, depending on the user. Adjustments can be made so that the ratio can be less than, equal to and/or greater than 0.7, 0.75, 0.8, 0.85, 0.9, 0.95, 1.0, 1.05, 1.1, 1.15, 1.2 or 1.25 or any value or range of values there between in 0.01 increments, all for a given shoe/foot within a shoe.

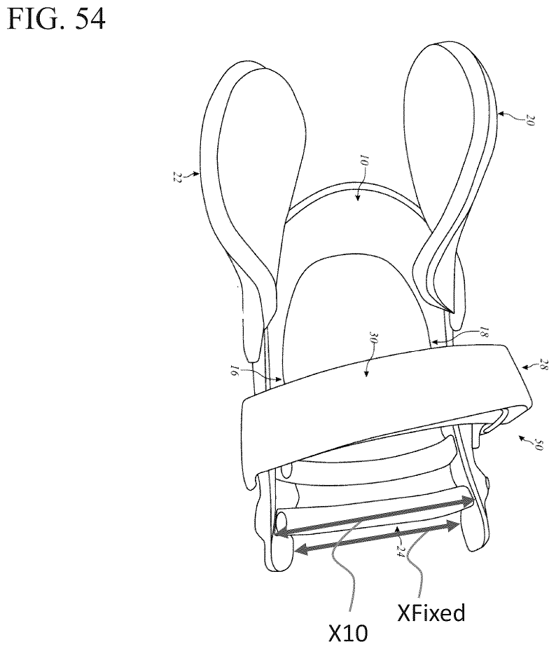

FIG. 54 schematically illustrates how the fixed bottom strap creates a fixed value of XFixed when these measurements are applied to the embodiment of FIG. 5 for a given shoe.

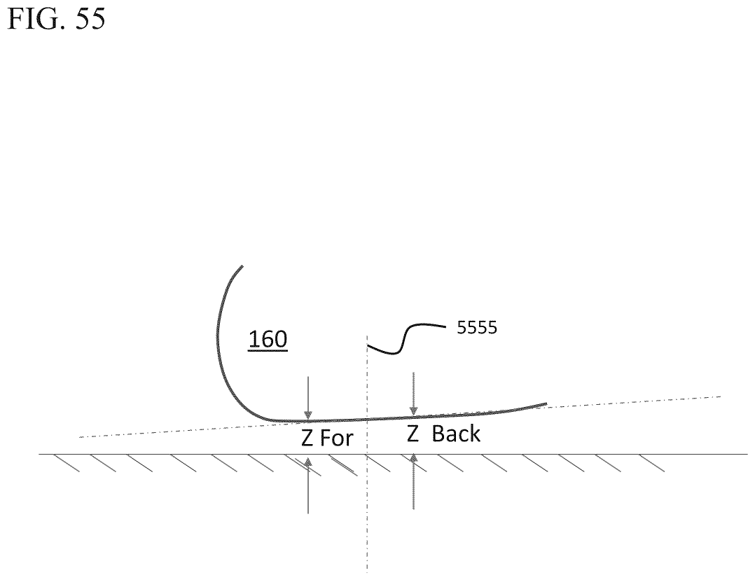

By utilizing an adjustable bottom strap as opposed to a fixed strap, the distance of the bottom of the sidewalls from the ground (or from the bottom of the sole of the shoe) can be varied for a given shoe or otherwise can be more controlled relative to the embodiments that utilize a fixed strap. In this regard, FIG. 55 depicts a portion of the outer contour of the lateral sidewall 160 relative to a level floor. Axis 5555 represents the centerline of the strap 260 when viewed from the side. In an exemplary embodiment, the distance from the bottom of the lateral sidewall to the floor when measured in the plane of axis 5555 and/or the dimension Z For and/or the dimension Z Back which are distances measured at distances that are 0.25, 0.3, 0.35, 0.4, 0.45, 0.5, 0.55, 0.6, 0.65, 0.7 and/or 0.75 inches forward and backwards from the axis 5555 (and the distances need not be the same) can vary with the same amount of tautness for the same size shoe and/or standard and/or foot. This in contrast to that which would be the case for the fixed strap. In an exemplary embodiment, the values for Z For and Z Back and Z at the axis 5555 (and they can be the same or different) can be 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4 or 1.5 inches or any value or range of values therebetween in 0.001 inch increments for the same size shoe and/or foot and/or standard such as a standard size 5, 6, 7, 8, 9, 10, 11, 12 or 13 shoe of USA measurements (herein, any standard used herein can be a standard male size 9 shoe or sneaker worn by a properly fitted foot). And Z For can be different from Z Back. The values can also be the case for the medial sidewall, and note that the values need not be the same for the lateral and medial sidewall. In an embodiment, Z For and/or Z Back is the closest that one or both sidewalls come to the flat ground when worn properly.

While the values and dimensions herein are often presented in terms of absolute values, in other embodiments, the values and dimensions can be considered in terms of percentages or ratios. In this regard, with respect to the aforementioned example of the Z values, the values for Z for example could be increased to an amount up to 750% from the smallest/lowest value (1.5 inches divided by 0.2 inches). Accordingly, any disclosure herein of any dimension or value that is adjustable or has a range corresponds to a disclosure in terms of a non-dimensional disclosure based on the lowest and/or the highest value and or any value within those ranges. Thus, in an exemplary embodiment, any of the Z values of FIG. 55 can correspond to an increase to an amount that is 125%, 150%, 175%, 200%, 225, 250, 275, 300, 325, 350, 375, 400, 425, 450, 475, 500, 525, 550, 575, 600, 625, 650, 675, 700, 725 and/or 750% from the smallest value possible.

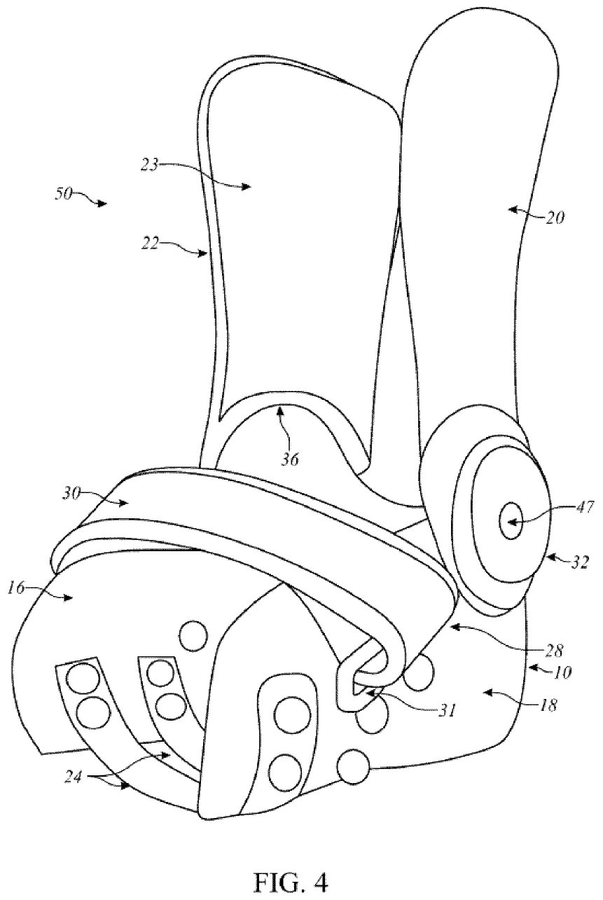

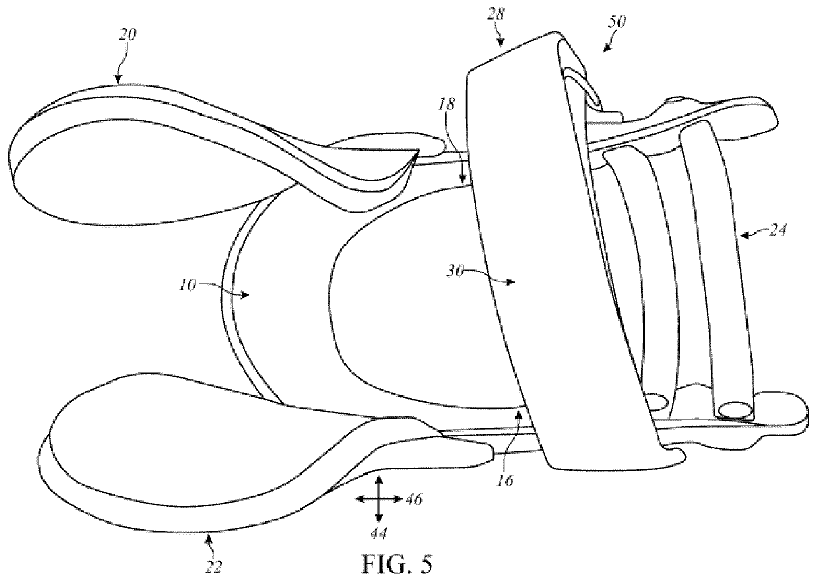

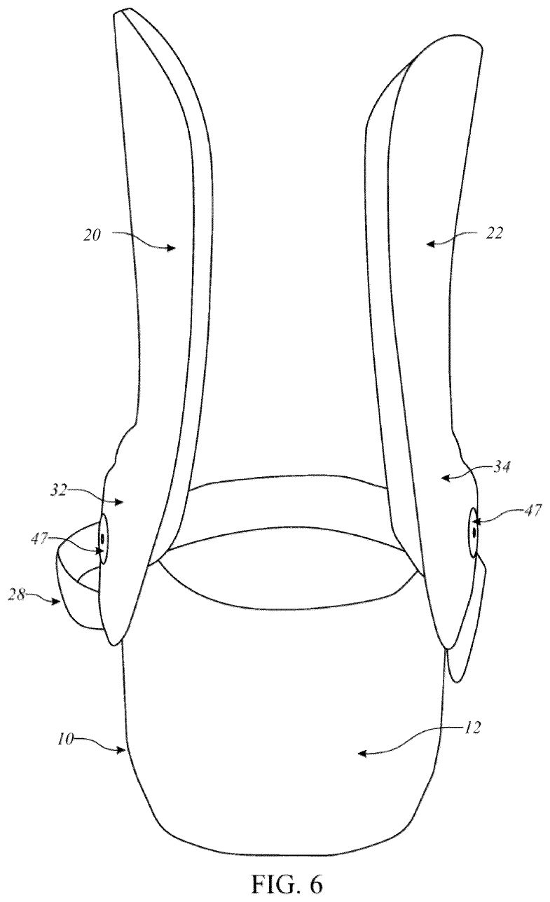

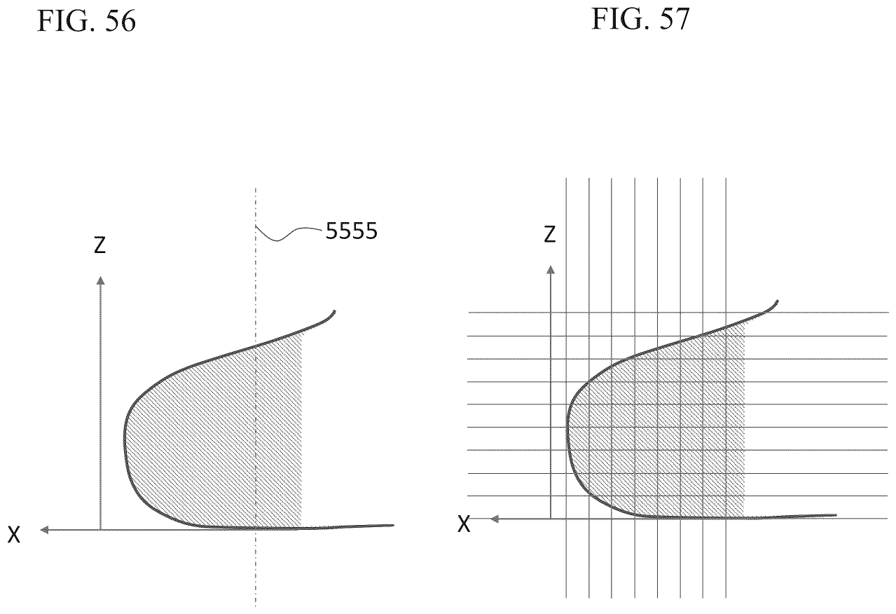

Embodiments that utilize the adjustable lower strap 260 can result in a more uniform and/or an increase in the overall friction force of the sidewalls on to the shoe worn with the external ankle brace. Referring to FIG. 56, there is shown a perfect side view of the lateral wall or the medial wall. The crosshatched area can be an area of measurement. In an exemplary embodiment, this section can be the entire forward section forward of the centerline 5555 of the lower strap 260. As shown in FIG. 6, the crosshatched area also includes some of the area in back of the centerline 555. In an exemplary embodiment, the area under consideration can extend 0.25, 0.5, 0.75, 1, 1.25, 1.5, 1.75 or 2 inches for example to the rear of centerline 5555. The rearmost boundary would be parallel with the centerline 5555. In an exemplary embodiment, utilizing the same standard shoe for the same amount of tautness in the straps, the friction force in the area in question can be more uniformly distributed and/or can be increased relative to that which would be the case with the nonadjustable bottom strap. By way of example only and not by way limitation, on a per unit area where the friction forces are measured in quarter inch square areas within the shaded area (FIG. 57 for example, where partial size quarter inch squares can be weighted or discounted (e.g., eliminated from the calculation, the average deviation (mean and/or median)) the friction force in one or more (1, 2, 3, 4, 5, 6, 7, 8, 9, 10, 11, 12, 13, 14 or 15, 16, 17, 18, 19, 20, 21 or 22 or more) of the square areas from the overall total friction force of the shaded area can be more than and/or equal to 10, 15, 20, 25, 30, 35, 40, 45, 50, 55, 60, 65, 70, 75 or 80% or any value or range of values therebetween in 1% increments less than (or greater than in some embodiments) that which is the case for the fixed lower strap for the same amount of tautness on the same standard (again, all things being equal). And note that there is no dichotomy between reducing the friction force at one area and an increase in the overall friction force, as this can be a result of more uniformly distributing the friction force. Where quarter inch squares have been utilized for the above example, squares of other size, such as 8^{th} inch squares or ⅜^{th} inch squares, or half inch squares, etc., can be utilized, or the above percentage values would still be indicative of the embodiments utilizing the adjustable strap. Any control subarea that can utilize to evaluate the adjustable strap.

FIG. 58 depicts another concept for measuring friction force. Here, circular areas having defined distances from the centerline 5555 of the bottom strap and from the side/edges of the sidewalls can be utilized as the areas in which the friction forces are measured in a manner concomitant with the above. In an exemplary embodiment, the circular areas can have a radius of 0.1, 0.15, 0.2, 0.25, 0.3, 0.35, 0.4, 0.45, 0.5, 0.55, 0.6, 0.65, 0.7, 0.75 inch or any value or range of values therebetween in 0.001 inch increments.

And it is noted that while the embodiments disclosed above focus on measuring the friction forces all the way from the front forward edge of the sidewalls, in other embodiments, consistent with the embodiment where the rearward extension of the shaded area from the centerline 5555 is limited, so can the forward extension of the shaded area be limited. It can be limited to any of the values detailed above for which the rearward extension is limited, and the forward extension need not be the same as the rearward extension.

And in an alternate embodiment, a total friction force can be measured to determine the difference between the fixed lower strap and the adjustable lower strap. In an exemplary embodiment, for the same size shoe and for the same standard and for the same tautness (all tautnesses are measured in the straps), again, all things being equal, for that shoe or standard fixed at the forward toe or at a location in front of the straps, the amount of force applied to the rigid heel enclosure in a rearward direction (with the upright extensions removed—this is purely a test of the friction forces associated with the rigid heel enclosure and the shoe) to create a 0.125, 0.25, 0.375 and/or a 0.5 inch gap (or an increase if a gap was present, which should not be) between the rear portion of the shoe and the rear portion of the rigid heel enclosure on the inside (or that size increase in the gap) can be measured. In an exemplary embodiment, the force required to create that gap can be equal to and/or greater than 105, 110, 115, 120, 125, 130, 135, 140, 145, 150, 155, 160, 165, 170, 180, 190, 200, 225, 250, 275 or 300% or more or any value or range of values therebetween in 1% increments than the force required to create that gap with the nonadjustable strap such as that detailed above in FIGS. 1-13.

In an exemplary embodiment, utilizing the same standard shoe for the same amount of tautness in the straps, the friction force in the total shaded area can be increased relative to that with the fixed lower strap. By way of example only and not by way limitation, the friction force in the shaded area can be increased 5, 10, 15, 20, 25, 30, 35, 40 or 45% or any value or range of values therebetween in 1% increments over that which is the case for the fixed lower strap for the same amount of tautness on the same standard (again, all things being equal).

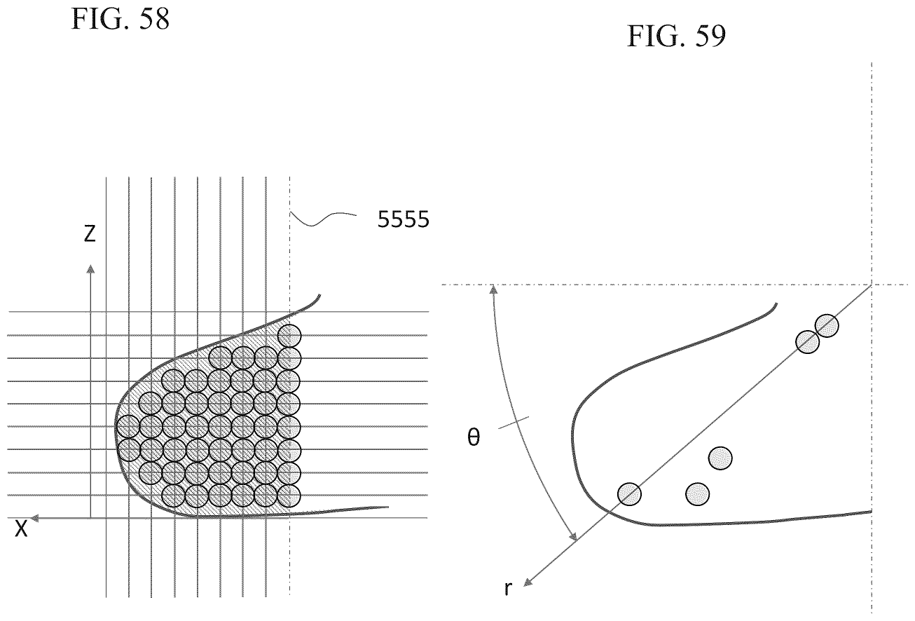

And it is noted that while the strap 260 centerline has been used as the basis for the locational measurements above, in other embodiments, other references can be used, such as, for example, the longitudinal axis of the through hole in the male medial joint portion and a plane flying on in parallel to that longitudinal axis, and parallel to the Z axis. Also, it is noted that while the embodiments have been described in terms of the two dimensional Cartesian coordinate system, other important systems, such as a polar coordinate system can be used. In this regard, by way of example, FIG. 59 depicts a polar coordinate system centered about the longitudinal axis of the through hole for the Chicago screw in the male joint portion. It can be seen how based on the angle from the horizontal/X direction and/or from the vertical/Z direction, and the distance (r) of the center of a circle (or square), the reference areas can be calculated or otherwise controlled. In an exemplary embodiment, r values can be 1.5, 1.75, 2, 2.25, 2.5, 2.75, 3, 3.25, 3.5, 3.75, 4, 4.25, 4.5, 4.75, 5, 5.25, 5.5, 5.75 or 6 inches or any value or range of values therebetween in 0.001 inch increments. Theta can be 30, 35, 40, 45, 50, 55, 60, 65, 70, 75 or 80 degrees or any value or range of values therebetween in 0.1 degree increments. The size of the circles can be those detailed above. Note that square areas can be used instead, the squares centered along the r and theta values.

While the embodiments above have focused on friction forces, other embodiments can focus on pressure. In an exemplary embodiment, the features associated with the aforementioned friction forces can be instead associated with pressures. By way of example only and not by way limitation, on a per unit area where pressure is measured in quarter inch square areas within the shaded area (FIG. 57 for example, where partial size quarter inch squares can be weighted or discounted (e.g., eliminated from the calculation, the average deviation (mean and/or median)) for the pressure in one or more or all of the square areas from the overall total pressure of the shaded area can be more than and/or equal to 10, 15, 20, 25, 30, 35, 40, 45, 50, 55, 60, 65, 70, 75 or 80% or any value or range of values therebetween in 1% increments lower (or greater in some other embodiments) than that which is the case for the fixed lower strap for the same amount of tautness on the same standard (again, all things being equal). And note that the values need not be the same for each square/circle.

In an exemplary embodiment, utilizing the same standard shoe for the same amount of tautness in the straps, the pressure in the total shaded area can be increased relative to that with the fixed lower strap. By way of example only and not by way limitation, the pressure in the shaded area can be increased 5, 10, 15, 20, 25, 30, 35, 40 or 45% or any value or range of values therebetween in 1% increments over that which is the case for the fixed lower strap for the same amount of tautness on the same standard (again, all things being equal).

And it is also noted that instead of using the total shaded area as the baseline, the sum total of the individual sub-areas can instead be used (thus alleviating any need to calculate the friction force/pressure in the space between the circles).

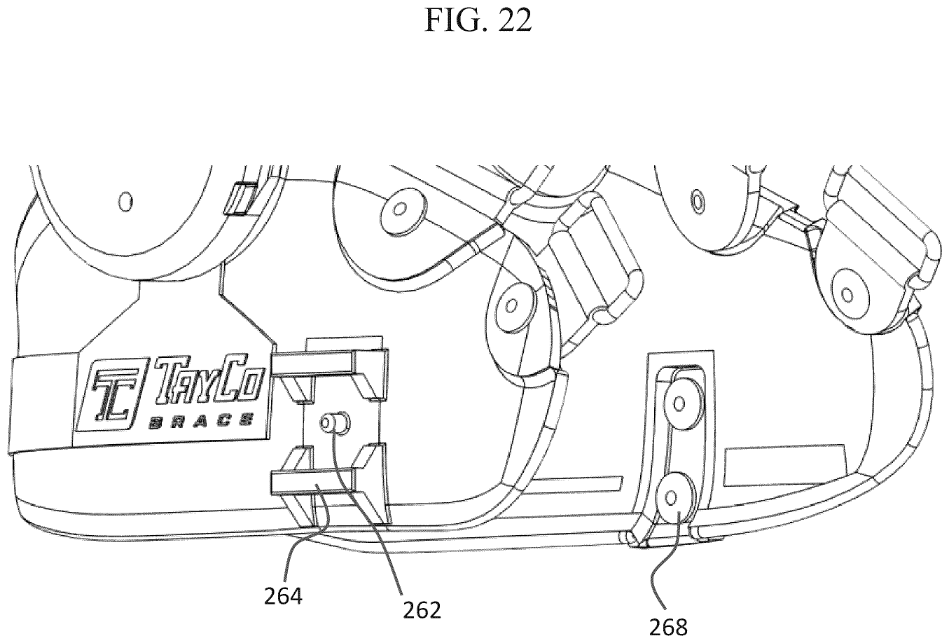

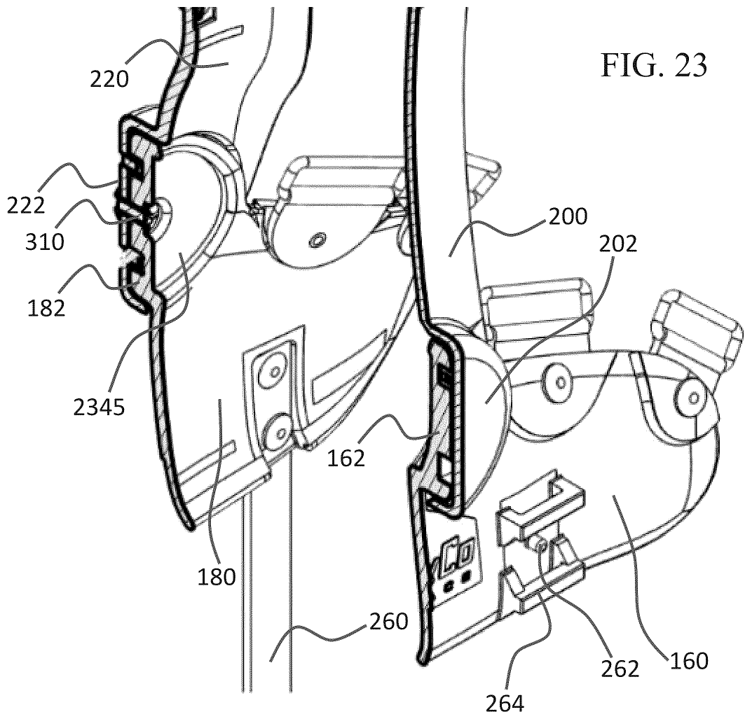

FIG. 23 depicts a cross-sectional view of the external ankle brace 500 on a plane lying on and parallel to the rotation axis of the medial joint and/or the longitudinal axis of the Chicago screw 310 that establishes the pivot of that joint. Owing to the offsets of the medial and lateral joints as will be described in greater detail below, the cross-section does not extend through the axis of rotation of the lateral joint and/or the longitudinal axis of the Chicago screw of that joint (more on this below). The cross-sectional view shows that the lateral and medial joints are made up of a male portion (respectively 162 and 182) that is monolithic with the bulk of the structure that makes up the respective sidewalls, and a female portion (respectively 202 and 222) that is monolithic with the bulk of the structure that makes up the respective uprights.

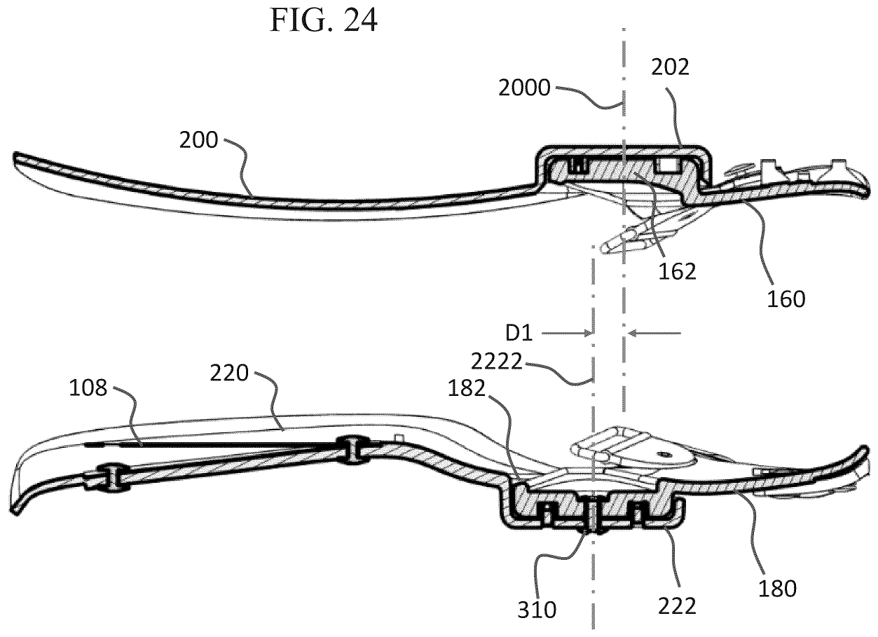

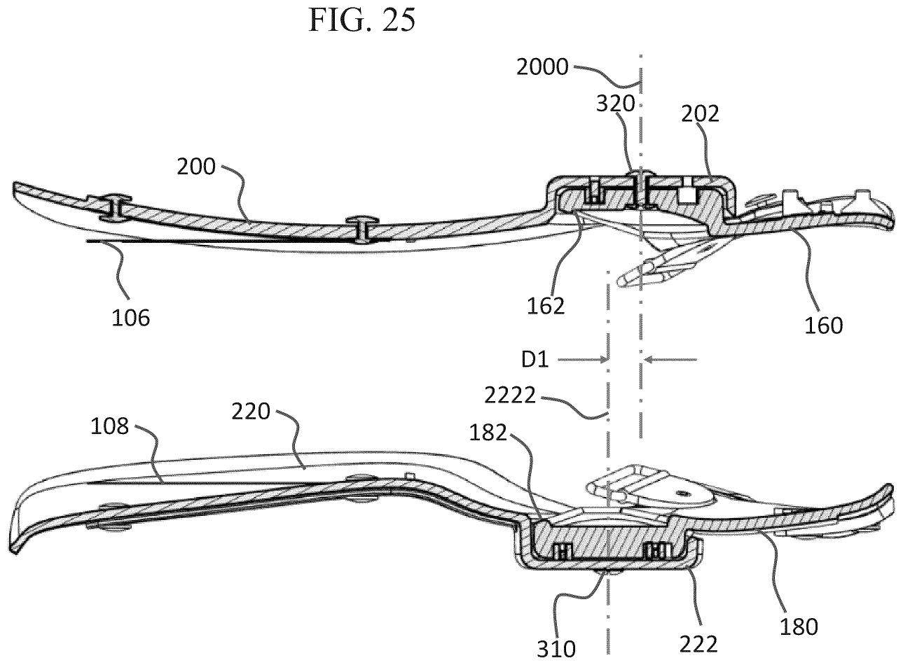

In an exemplary embodiment, on a per mass and/or per volume basis, the respective male and female portions are monolithic with structure that makes up at least 70, 75, 80, 85, 90, 95, 96, 97, 98 or 99 or 100% of the respective upright and/or the respective sidewall and/or the rigid heel enclosure structure (the control would not include the straps or the rivets, etc.—the control would include for example the reinforcing structures such as elements 108, 106 and/or 110). In an exemplary embodiment, the joint portions are monolithic with a structure of the respective sidewalls and/or heel enclosure and/or the uprights, which structure extends in a monolithic manner from the bottom most portion of that component to the top most portion of that component and/or from the forward most portion of that component to the rearward most portion of that component and/or a distance that is 70, 75, 80, 85, 90, 95, 96, 97, 98 or 99 or 100% of the overall topmost distance and/or the forward and/or reward-most distance. By way of example only and not by way limitation, FIG. 24 depicts the cross-sections of the bulk of the structure of the uprights (the crosshatched portions with major components extending horizontally—this excludes the element 108 and the rivets therefor and the Chicago screw 310 for example) and the bulk of the structure of the sidewalls and the rigid heel enclosure. FIG. 25 depicts the cross-sections of the bulk of the structure of the uprights (excluding the element 106 and the rivets therefore and the Chicago screw 320 for example) and the bulk of the structure of the sidewalls and the rigid heel enclosure. As seen in FIG. 24 with respect to the lateral upright extension 200, the cross-section extends unbroken from the top to the bottom most portion thereof, the structure that establishes such extension is monolithic with the female portion 202 of the lateral joint. As seen in FIG. 24 with respect to the lateral sidewall 160, the cross-section extends unbroken from the top to the bottom most portion thereof, the structure that establishes such extension is monolithic with the male portion 162 of the lateral joint. As seen in FIG. 25 with respect to the medial upright 220, the cross-section extends unbroken from the top to the bottom most portion thereof, the structure that establishes such extension is monolithic with the female portion 222 of the medial joint. As seen in FIG. 25 with respect to the medial sidewall 180, the cross-section extends unbroken from the top to the bottom most portion thereof, the structure that establishes such extension is monolithic with the male portion 182 of the medial joint. But again, in an exemplary embodiment where the structure is not monolithic, the distance could be 70 or 80 or 90% as detailed above. And note that the just described features are not excluded because there are rivets for example in the lateral upright as shown in the cross-section of FIG. 25. Because the structure extends monolithically about those rivets, the aforementioned features are still present. That is, the distance need not lie in an exact plane to meet the aforementioned features. Still, by taking the cross-section in a plane that is offset and otherwise away from the rivets and/or Chicago screw, one can have an unbroken cross-section from the top to the bottom that includes the hinge portions as seen in FIGS. 24 and 25.

It is noted that in an exemplary embodiment, a circular foam pad is placed on the inboard face 2345 of the male joint portions so as to pad between the joint portion and the respective malleolus (or, more accurately, the skin over the respective malleolus).

It is noted that in an exemplary embodiment, there is a washer that is located between the faces of the male and female portions that are normal to the pivot axis. The washers has a hole through which the Chicago screws extend. The washers can be low friction washers that are made from a polymer or any other material that is suitable. In an exemplary embodiment, the washers maintain a space between the aforementioned faces of the male and female joint portions that is at least and/or equal to and/or no more then 0.005, 0.01, 0.015, 0.02, 0.25, 0.03, 0.04, 0.05, 0.06, 0.07, 0.08, 0.09, 0.1, 0.125, or 0.15 inches or any value or range of values therebetween in 0.0001 increments (or less), at least to achieve the standard English measurements. In an exemplary embodiment, the aforementioned values are the thickness of the washer. In an exemplary embodiment, the outer diameter of the washer (it can be circular outside diameter and or with respect to the inside diameter) and/or maximum outer diameter of the washer can be less than and/or equal to and/or greater than 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2, 2.25, 2.5, 2.75 or 3 inches or any value or range of values therebetween in 0.001 increments (or less) to achieve the English measurement units at least.

In an exemplary embodiment, the interfacing or otherwise facing surfaces of the male and female joint portions are circular, concomitant with the fact that the portions rotate relative to one another. It is also noted that in an exemplary embodiment, the figures are drawn to scale in totality and/or on a percent basis (e.g., if a view is shown in a shrunken manner, that is still to scale on a percent basis).

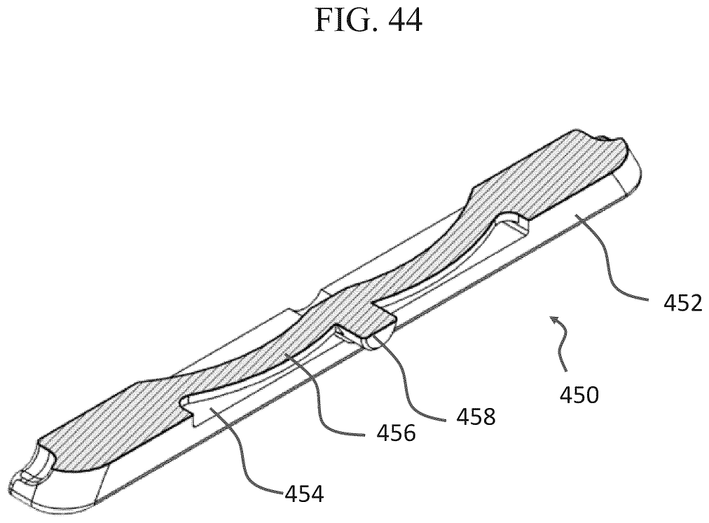

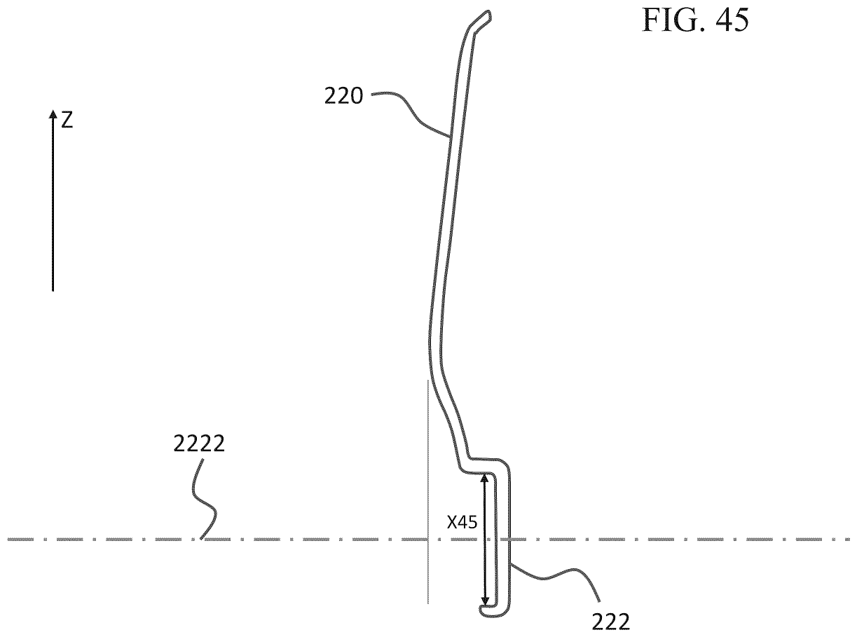

FIG. 45 shows an abbreviated cross-section of section D-D. As seen the diameter of the female portion normal to the axis of rotation 2222 of the medial upright extension can be 1.5, 1.75, 2, 2.25, 2.5, 2.75, 3, 3.25 or 3.5 inches or any value or range of values therebetween in 0.0001 inch increments. It is noted that the comparable outer diameter of the male joint portion can be, relative to X45, less than and/or equal to and/or greater than 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29 or 0.30 or more inches or any value or range of values therebetween in 0.0001 inch increments undersized relative to the female portion (so if X45 is 2.25 inches, the male outside diameter might be 2.10 inches). Wall the cross-section shown in FIG. 45 is for the female medial joint portion 222, this can also represent the dimensions for the female lateral joint portion (and the undersize detailed above for the male lateral joint portion).

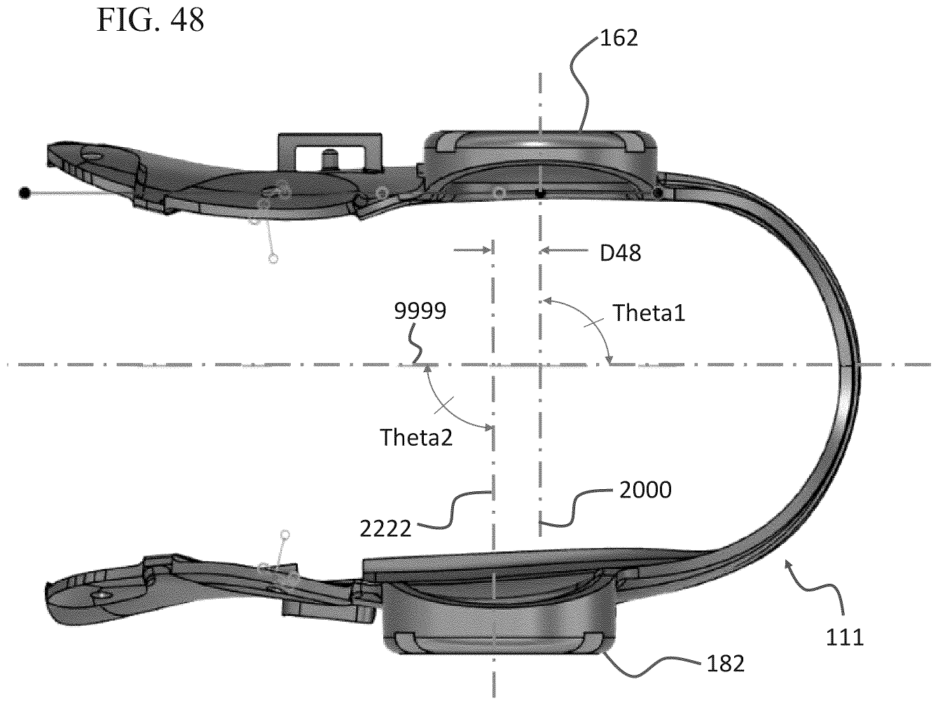

As can be seen in FIGS. 24 and 25, the respective axes 2222 and 2000 of joint rotation/joint pivot for the medial joint and the axial joint are offset by a distance D1 in the Z axis (the axis normal to the ground/bottom of the brace). In an exemplary embodiment, D1 can be less than and/or equal to and/or greater than 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9 or 2 inches or more or any value or range of values therebetween in 0.0001 increments (or less), at least to achieve the standard English measurements. With reference to FIG. 48, which shows a top view of the rigid heel enclosure 111 according to an exemplary embodiment, the respective axes can also be offset with respect to the X direction (forward-backwards—the direction of axes 9999), where D48 can be any of the values of D1, and the values for the offset in the X direction need not be the same as the values for the offset in the Z direction. (That is, we are not repeating the values of D1 for D48 in the interests of textual economy.) And note that in some embodiments, there may not be an offset in one or both of the dimensions.

And it is also noted that one or both of axes 2000 and axes 2222 can be at an oblique angle relative to axes 9999. In an exemplary embodiment, the angle Theta1 and/or Theta 2 (both shown as 90 degrees), can be less than, greater than and/or equal to 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 25, 16, 107, 109, 110, 111, 112, 113, 114, 115, 116, 117, 118, 119 or 120 degrees (plus or minus). The axes 2000 and 2222 can be parallel to each other or oblique to one another in the plane shown in FIG. 48 (plane normal to the Z axis). Theta1 need not be the same value (absolute and/or plus or minus) as Theta2. Theta1 can be the same as Theta2.

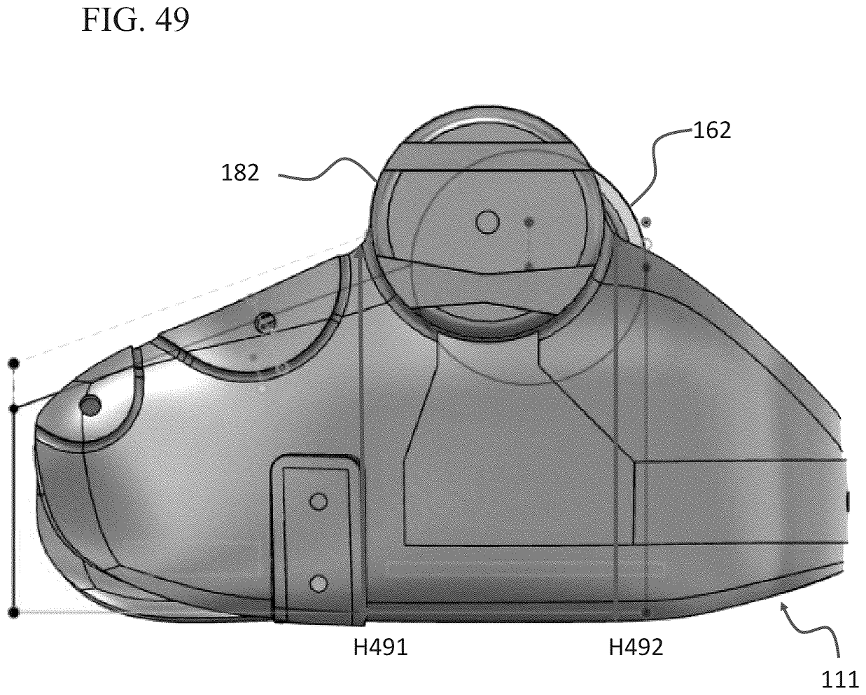

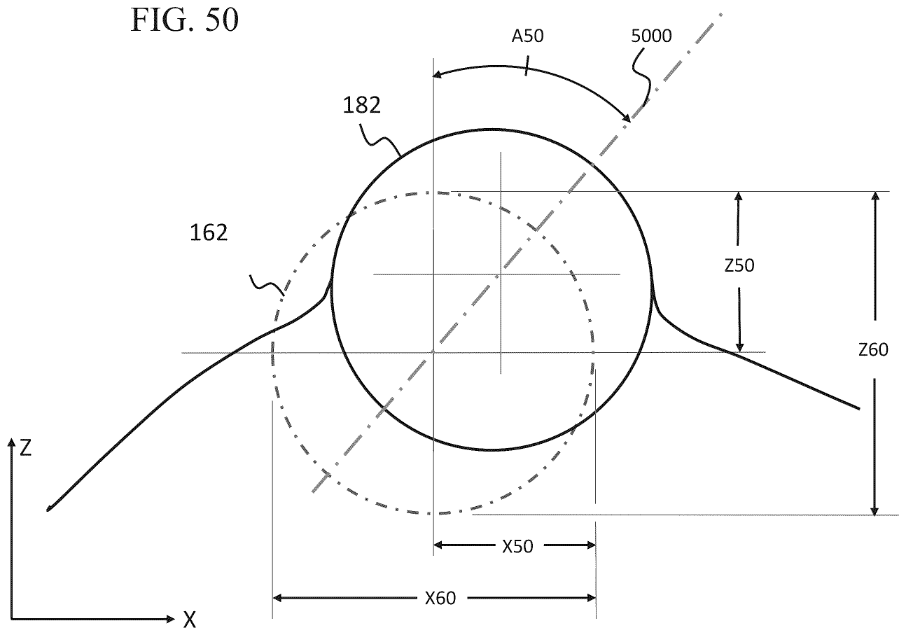

FIGS. 49 and 50 are side views of the rigid heel enclosure 111 for different sides of the foot. As can be seen, the male medial joint portion 182 is located higher and more forward than the male lateral joint portion 222, and thus the axis of rotation of the medial joint is higher and more forward than the axis of rotation of the lateral joint. FIG. 49 shows dimensions H491 and H492. These are the heights from the bottom of the sidewall to the top of the sidewall just forward and just behind the male portion 182 (between 0.01 and 0.25 inches from the forward most and/or rearward most portion of the male portion 182 in the X axis). In an exemplary embedment, H491 and/or H492 is less than and/or equal to and/or greater than 2.5, 2.75, 3, 3.25, 3.5, 3.75, 4, 4.25, 4.5, 4.75 or 5 inches or any value or range of values therebetween in 0.0001 inch increments. Again, this is to scale, so the other heights of the sidewall can be scaled.

And as can be seen, the heights of the medial sidewall are higher than those of the lateral sidewall, at least at the portions proximate the hinge portions. But still, referring to FIGS. 15 and 16, it can be seen that the overall profile of the lateral sidewall is lower than that of the medial sidewall, the difference being less than and/or equal to and/or greater than 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15 or 1.2 inches or any value or range of is therebetween in 0.0001 inch increments at one or more or all of the locations along the length of the sidewall (and the values need not be the same—the slopes could be different, and thus the differences could change along the length).

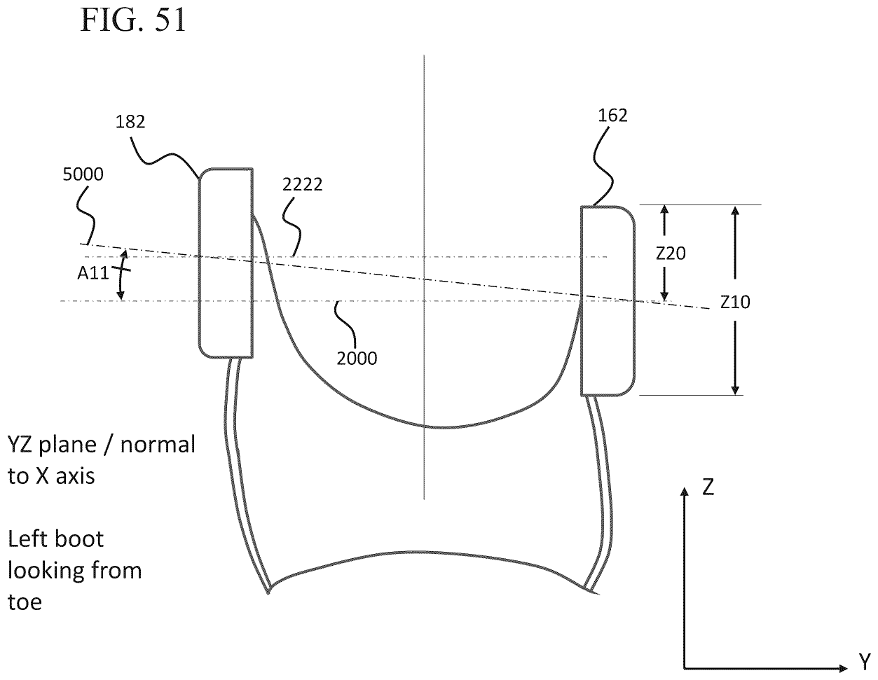

FIG. 50 and FIG. 51 (the latter showing a view of the rigid heel enclosure 111 looking down the X axis) present the axes 5000. In an exemplary embodiment, as shown in FIG. 51, the axis 5000 extends through the respective axes of rotation of the joints. In this exemplary way of describing the axis 5000, the location where it bisects the joint axes of rotation are at the outboard most locations of the male joint portions, or, more accurately, at extrapolated outermost portions thereof (because there are through holes at those locations for the Chicago screws that are utilized as the bearing surfaces in the X and Z direction (the washer is utilized for the bearing surfaces in the Y direction)). One can describe this extrapolated outermost portion as the tangent surface to the Y direction bearing faces of the male joint portions. In another exemplary embodiment, the axis 5000 bisects the joint rotation axes at the extrapolated innermost portions of the male joint portions or the comparable reference locations described in the preceding sentence. In an exemplary embodiment, the axis 5000 bisects the joint rotation axes in between those extrapolated surfaces, etc. In an exemplary embodiment, A11 and/or A50 can be less than and/or equal to and/or greater than (and they need not be the same, but can be) 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 degrees or any value or range of values therebetween in 0.1 degree increments (and note that angle A11 probably will not be some of the higher numbers just referenced (we are using groupings here for textual economy)). Z60 can be the OD of the male portion 162 detailed above, with Z50 has half that value. The cross-center of the axis of rotation for the respective male portions are shown.

While the male joint portions were utilized as the frame of reference for the aforementioned axis 5000, in an alternate embodiment, the female joint portions can be utilized as the frame of reference. For example, the outboard most portion (the extrapolated surface thereof) and/or the inboard most portion of the female joint portions or the middle, etc. (any comparable reference described for the male joint portion can be utilized in at least some exemplary embodiments as the location where the axis 5000 bisects the rotation axes).

It is also noted that in an exemplary embodiment, the rotation axis 2222 is not parallel with rotation axis 2000 and/or one or both of those axes are not parallel with the Y axis and/or not parallel to the XY plane and/or the YZ plane and/or the ZX plane. In an exemplary embodiment, one or both (and they need not be the same) is less than and/or equal to and/or greater than 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 degrees or any value or range of values therebetween in 0.1 degree increments from the XY plane and/or the YZ plane and/or the ZX plane (and the values related to the respective planes need not be the same values, but can be).

In an exemplary embodiment, the maximum and/or minimum distance in the Y direction/the direction of the rotation axes of overlap of the male and female “housings” can be less than and/or equal to and/or more than 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49 or 0.50 or more inches or any value or range of values therebetween in 0.0001 inch increments undersized relative to the female portion (so if X45 is 2.25 inches, the male outside diameter might be 2.10 inches). The measurements would be based on the female joint portion walls that laterally encircle (potentially only partially in some embodiments) the male portion/the radial wall that establishes the female portion.

In an exemplary embodiment, the axes of joint rotation/joint pivot are anatomically aligned, at least generally, with the respective medial malleus and the lateral malleus of a 50 percentile human factors engineering male and/or female having been born in the United States of America and/or the European Union and/or Japan on Aug. 15, 1961, 1971, 1981, 1991, 2001, 2011 or 2016, or 2017 or 2018 or 2019, and, for example, the age of that person being calculated from Aug. 15, 2021. By way of example only and not by way limitation, in an exemplary embodiment, the axes of rotation extend through the respective outboard most portions of the bone that establishes the medial malleus and the lateral malleus when the braces worn on a human and/or less than and/or equal to 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 or 1 inch from that point or any value or range of values therebetween in 0.001 increments. Again, consistent with any of the values detailed herein, unless otherwise specified, the values for one need not be the same as the other. Thus, the pivot axis of the medial joint could extend 0.13 inches from the outboard most portion of the medial malleolus bone and the pivot axis of the lateral joint could extend 0.28 inches from the outboard most portion of the lateral malleolus bone.

And it also that any one or more of the values detailed herein can be for a recipient specific person (i.e., the person who is actually utilizing the external ankle braces detailed herein—embodiments thus include an ankle brace being worn by specific person having one or more of the aforementioned teachings detailed herein).

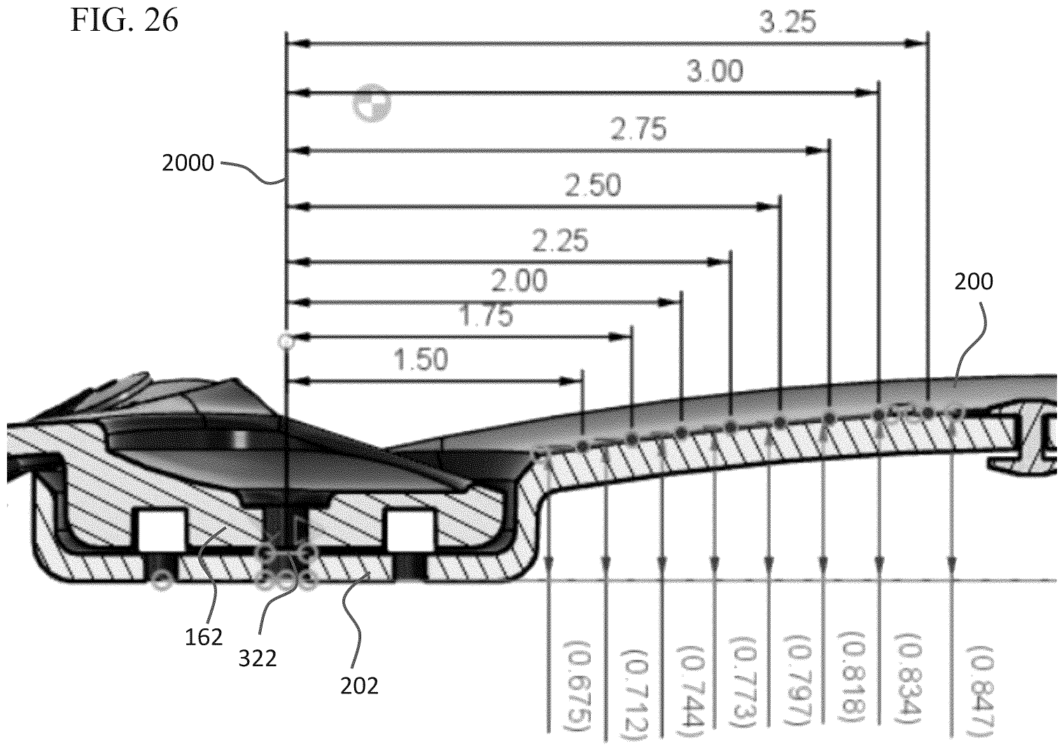

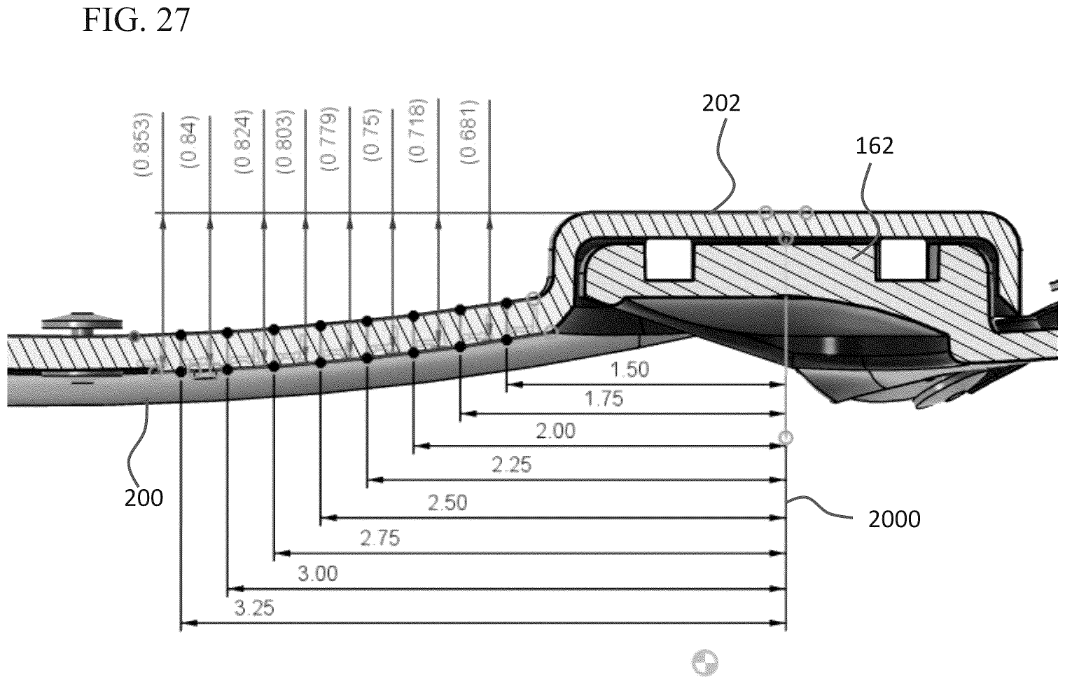

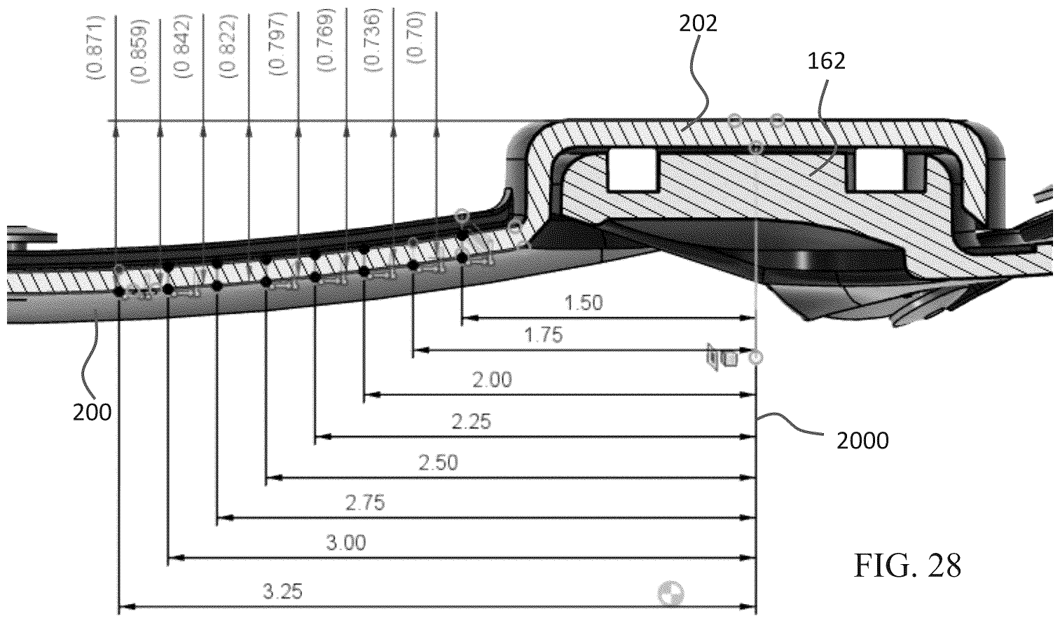

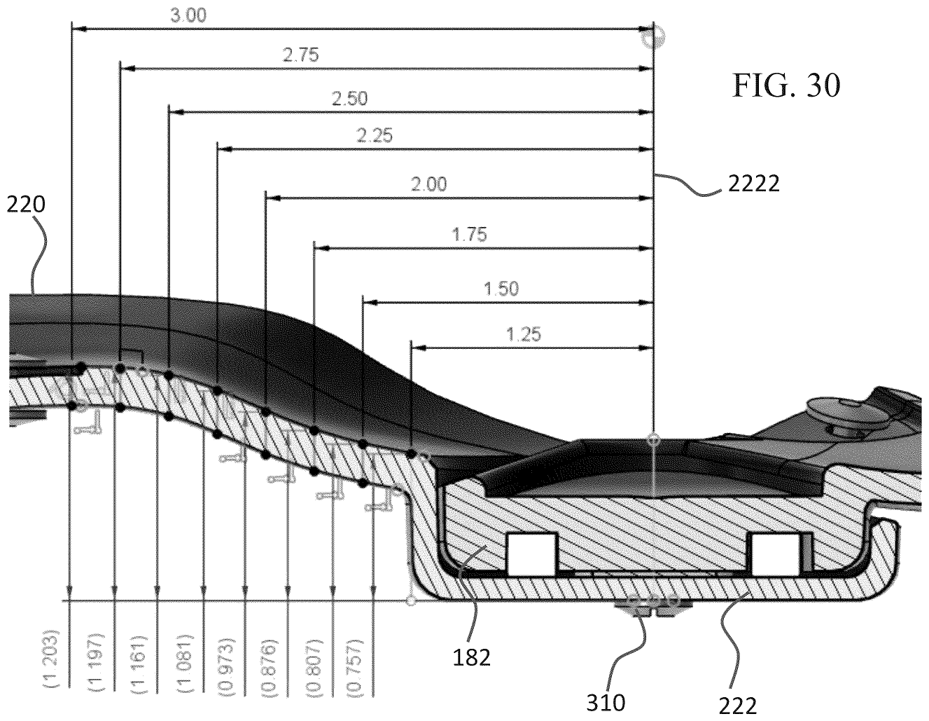

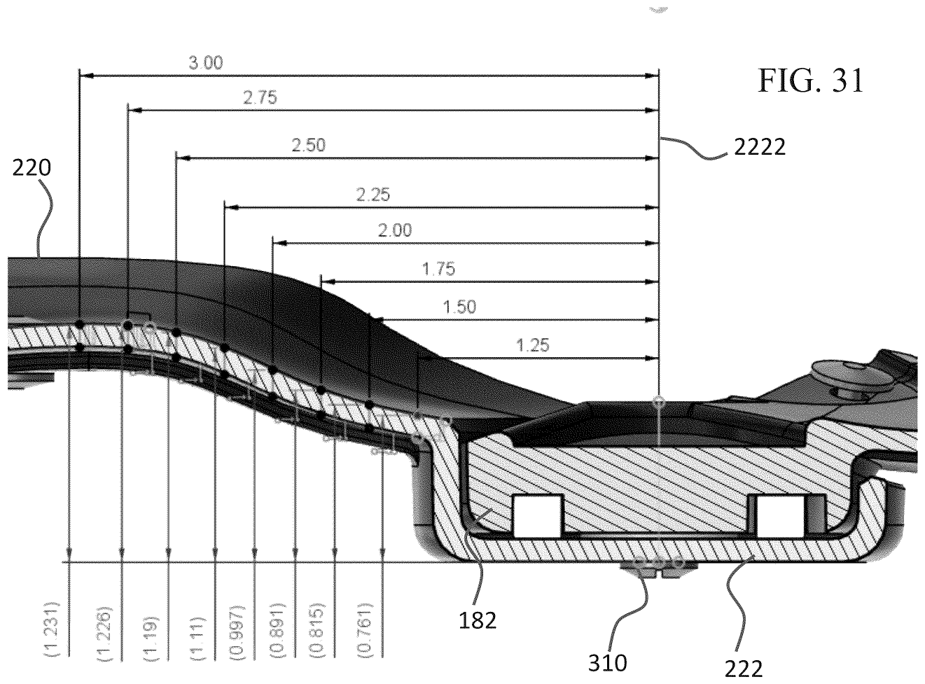

FIG. 26 to 31 show dimensioned cross-sectional views respectively corresponding to the cross-sections A-A to F-F of FIGS. 15 and 16. Cross-section A-A are taken through the axis of rotation/pivot of the lateral upright extension 200 and/or through the longitudinal axis of the respective Chicago screw, which cross-section lies on a plane that is normal to the bottom of the lateral sidewall when the lateral upright is at the 90° position and/or the medial joint is in the zero pivot state (the multi-mode pivot system is locked so that the lateral upright cannot pivot—more on this below) and/or normal to a flat level surface upon which the user of the external ankle brace would walk. Cross-section D-D is taken through the axis of rotation/pivot of the medial upright extension 220 and/or through the longitudinal axis of the respective Chicago screw, which cross-section lies on a plane that is normal to the bottom of the medial sidewall when the medial upright is at the 90° position and/or the medial joint is in the zero pivot state (the multi-mode pivot system is locked so that the medial upright cannot pivot—more on this below) and/or normal to a flat level surface upon which the user of the external ankle brace would walk. Cross-section B-B shown in FIG. 27 is on a plane parallel to and 0.25 inches behind that of cross-section A-A, and cross-section C-C shown in FIG. 28 is on a plane parallel to and 0.5 inches behind that of cross-section A-A. Cross-section E-E shown in FIG. 30 is on a plane parallel to and 0.25 inches behind that of cross-section D-D, and cross-section F-F shown in FIG. 31 is on a plane parallel to and 0.5 inches behind that of cross-section D-D.

It is noted that in an exemplary embodiment, cross-sections B-B, C-C, E-E and F-F are representative of cross-sections that would be in front of the cross-sections A-A and D-D, respectively, by the amounts detailed.

It is noted that the values of FIGS. 26 to 31 along with the structural features thereof associated there with can be utilized to scale other structural features disclosed herein in some embodiments. But it is noted that in some embodiments, the structures can have other dimensions and/or configurations.

In an exemplary embodiment, any dimension of any structure disclosed herein corresponds to an alternate disclosure where the dimensions of this structure in the alternate disclosure vary from those presented herein by less than and/or equal to and/or greater than 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 percent or any value or range of values therebetween in 0.01% increments where the disclosed dimension is the baseline. By way of example only and not by way limitation, this can be the case with respect to external ankle braces that are utilized for toddlers, where the values disclosed herein are for an external ankle brace that is used for a person wears a standard male size 9 shoe (USA) and/or a size 5, 6, 7, 8, 10, 11, 12 or 13 shoe. Further by way of example only and not by way limitation, this can be the case with respect to an external ankle brace that is utilized for someone who wears a male size 13 shoe. Also by way of example, this can be the case for someone who wears a male size 9 shoe but where the embodiment has a different shape for whatever reason (accommodate a different anatomical ankle, for example).

It is noted that while the cross-sections presented in FIG. 26 to 31 stop at 0.5 inches from behind the pivot points, it is to be understood that there will be contours that can be located at cross-sections further behind (and thus further forward) than those cross-sections presented. In an exemplary embodiment, the contours for the cross-sections at 0.75 inches and at 1 inch and at 1.25 inches and at 1.5 inches behind and or in front of the planes from the pivot points can have the extrapolated values from the values disclosed herein. In an exemplary embodiment, linear extrapolation can be utilized and/or a curve fit can be utilized (cubic (such as obtained via a cubic spline), simple linear, quadratic, polyratio (1,1), (2,2), or any other curve fitting technique that can be utilized, etc.), and those that can be accomplished utilizing three points. Also, values in between these cross-sections can be extrapolated, also utilizing linear and or curve fitting techniques, such as curve fitting techniques that can be applied utilizing three points. Cubic spline interpolation can be used. And note that in at least some exemplary embodiments, the values in the Z direction are the control values. Thus, in an exemplary embodiment, any disclosure herein corresponds to an alternate disclosure of values for the not specifically stated values just detailed correspond to those that are obtained via the linear interpolation and/or the curve fitting techniques and/or the cubic spline interpolation techniques.

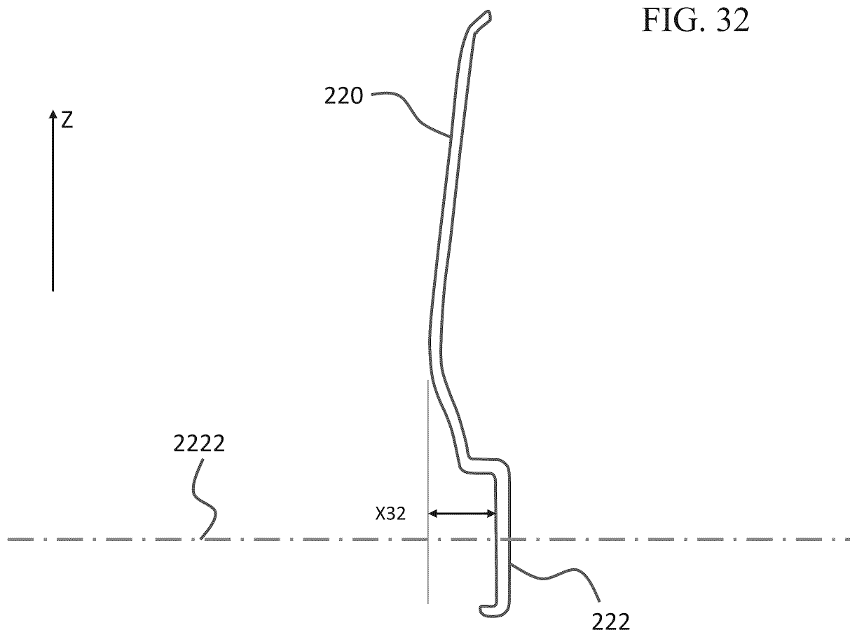

FIG. 32 depicts an abbreviated cross-section of the medial upright extension 220, which cross-sections taken on a plane lying parallel to and on the medial joint axis 2222, and also lying parallel to the Z direction of the overall brace. As can be seen, there is a maximum value X32 which corresponds to the maximum distance from the most outboard surface that faces inboard (the inboard face of the female joint portion 222 that is normal to the axis 2222) and the most inboard surface that faces inboard. In an exemplary embodiment, X32 is greater than, less than, and/or equal to 0.4, 0.45, 0.5, 0.55, 0.6, 0.65, 0.7, 0.75, 0.8, 0.85, 0.9, 0.95, 1, 1.1, 1.2, 1.3, 1.4 or 1.5 inches or any value or range of values therebetween in 0.01 increments.

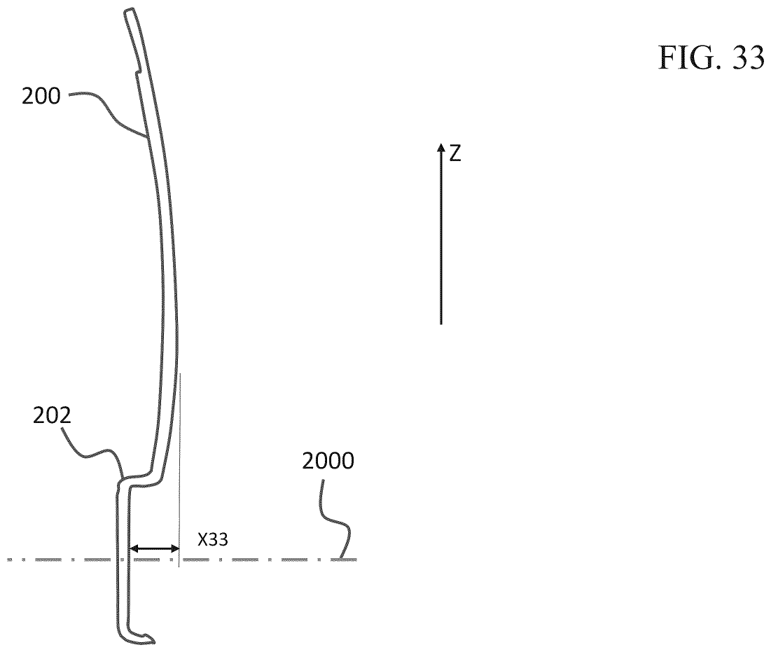

FIG. 33 depicts an abbreviated cross-section of the medial upright extension 200, which cross-sections taken on a plane lying parallel to and on the medial joint axis 2000, and also lying parallel to the Z direction of the overall brace. As can be seen, there is a maximum value X33 which corresponds to the maximum distance from the most outboard surface that faces inboard (the inboard face of the female joint portion 202 that is normal to the axis 2000) and the most inboard surface that faces inboard. In an exemplary embodiment, X33 is greater than, less than, and/or equal to 0.4, 0.45, 0.5, 0.55, 0.6, 0.65, 0.7, 0.75, 0.8, 0.85, 0.9, 0.95, 1, 1.1, 1.2, 1.3, 1.4 or 1.5 inches or any value or range of values therebetween in 0.01 increments. In an exemplary embodiment, X33 is different from X32.

And as can be inferred from the above, embodiments include a medial upright extension that has an interior surface that has contours that are distinctly different from those of the lateral upright extension, at least with respect to locations within a certain radius from the respective pivot axes. In an exemplary embodiment, as can be seen, the cross-section of the medial upright extension has a concave portion (with a frame of reference facing inboard) just above the female portion of the joint, and then the cross-section transitions to a convex portion (again with a frame of reference facing inboard), and then the radius of curvature of this convex section changes to then curve upwards and outwards with increasing location in the Z direction. This is contrasted to a corresponding cross-section of the lateral upright extension, which as constant convex cross-section (with a frame of reference facing inboard) with increasing Z direction. This complex and interchanging curvature of the medial upright extension has utilitarian value with respect to accommodating the medial malleus of a 50 percentile human factors engineering male and/or female according to those detailed herein.

Embodiments can include a multistage pivot system that has at least three states: a first state where one or both of the uprights cannot pivot relative to the respective sidewalls to which they are attached (and in an exemplary embodiment constitutes the 90° direction from the bottom of the rigid heel enclosure and/or from the level surface upon which a person walks when utilizing the brace); a second state which permits one or both of the uprights to pivot relative to the respective sidewalls forward and/or backwards by respective amounts (which respective amounts could be the same or different backwards relative to forward, and can be different for each upright); and a third state that limits pivoting forward and/or backwards toward amount that is less than the forward and/or backward amounts of the second state.

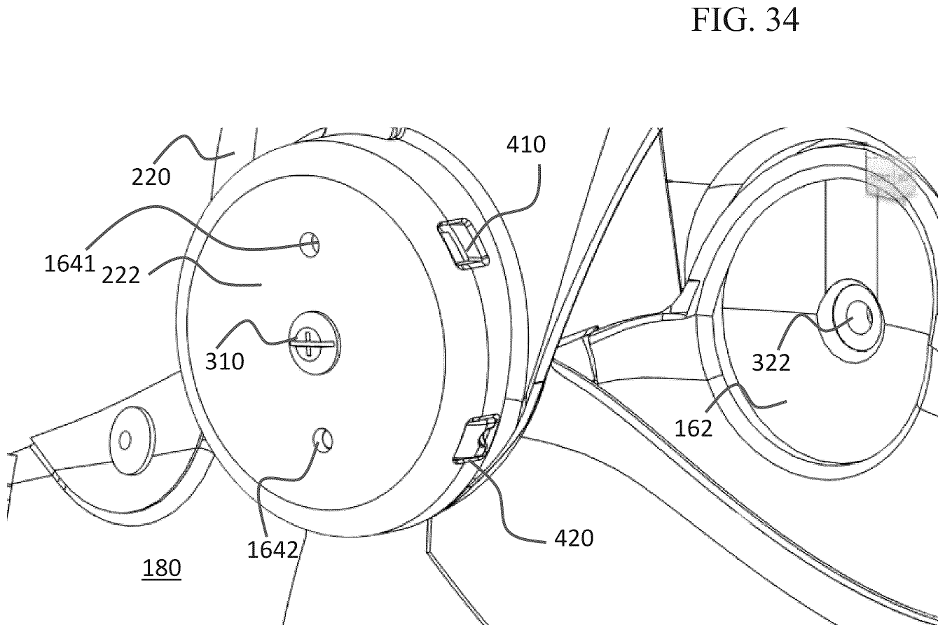

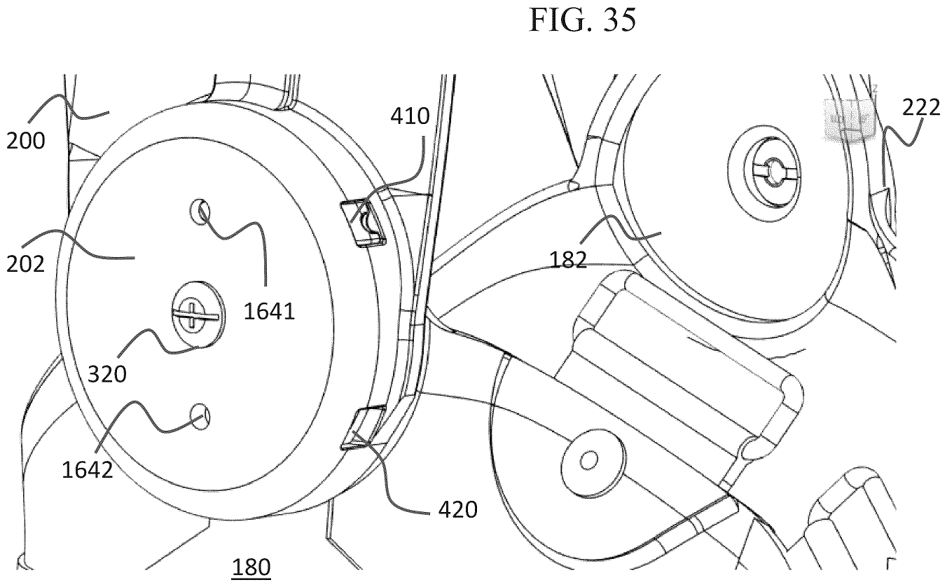

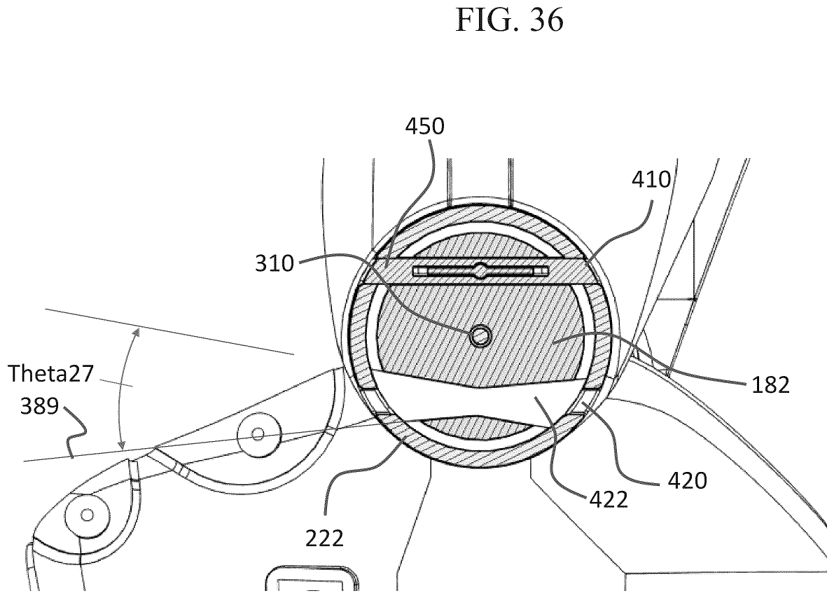

FIGS. 34-39 depict various views associated with the medial joint and lateral joint according to an exemplary embodiment, and a multi-stage pivot arrangement. FIGS. 34 and 35 present an isometric view of the medial and lateral joints according to an exemplary embodiment. As can be seen, there are openings 410 and 420 in the sidewalls of the female medial joint portion 222 and corresponding openings in the sidewalls of the female lateral joint portion 162. These openings enable lock bar 450 to be inserted into respective joints. FIGS. 36 and 37 show a cross-sectional view of the medial joint. These figures show the passage 412 and 422 through the male medial joint portion 182. As can be seen, there is specific alignment with passage 412 and openings 410, and general alignment with passage 422 and openings 420.

FIG. 36 shows the lock bar 450 in passageway 412 and openings 410. This prevents the medial upright extension 220 from pivoting owing to the contact of the lock bar 450 with the surfaces of passage 412 through the male medial joint portion 182 and the surfaces that established the opening 410 in the sidewall of the female medial joint portion 222. This is roughly analogous to sticking a pipe between the spokes of a bicycle wheel where the pipe cannot move in any direction except axially. It is noted that in an exemplary embodiment, the lock bar need only contact one of the openings 410. Indeed, in this exemplary embodiment, the reason why there are two openings 410 is to enable the lock bar to be both inserted and removed utilizing a pushing action. The lock bar 450 can be pushed into the position shown in FIG. 36 to lock the upright, and then pushed further in that same direction to remove the lock bar and thus enable the upright to pivot.

And FIG. 36 shows the angles of passageway 422, which angles permit the movement while limiting the overall movement. Here, Theta27 can be less than greater than and/or equal to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 degrees or any value or range of values therebetween in 0.1 degree increments. Consistent with having different forward angular movement than backward angular movement, the lower boundary of the angle (reference 389) can be an angle that is less than greater than and/or equal to 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 40 degrees or any value or range of values therebetween in 0.1 degree increments from the bottom surface or the top surface of the lockbar 420 when present when the top lockbar 410 is present, thus locking the upright.

FIGS. 37 and 38 depict the lock bar 450 in passageway 422 and openings 420. This prevents the medial upright extension 220 from pivoting the full amount forward and/or the full amount backward owing to the contact of the lock bar 450 with the surfaces of passage 422 through the male medial joint portion 182 and the surfaces that established the opening 420 in the sidewall of the female medial joint portion 222. This is roughly analogous to sticking a pipe between the spokes of a bicycle wheel where the pipe can move a bit about the axis of rotation but not too much. It is noted that in an exemplary embodiment, the lock bar need only contact one of the openings 420. Indeed, in this exemplary embodiment, the reason why there are two openings 420 is to enable the lock bar to be both inserted and removed utilizing a pushing action. The lock bar 450 can be pushed into the position shown in FIG. 37 to lock the upright, and then pushed further in that same direction to remove the lock bar and thus enable the upright to pivot.

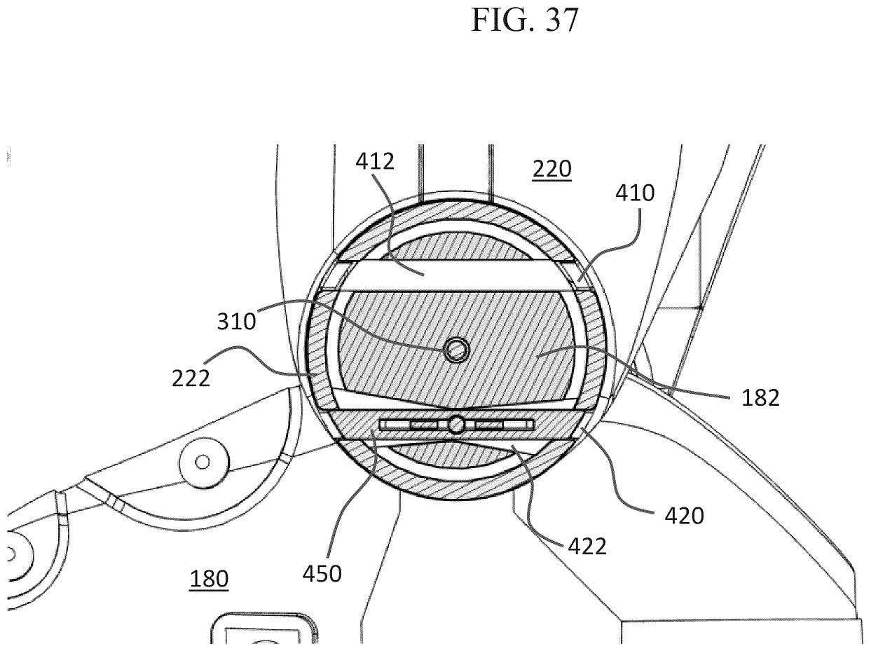

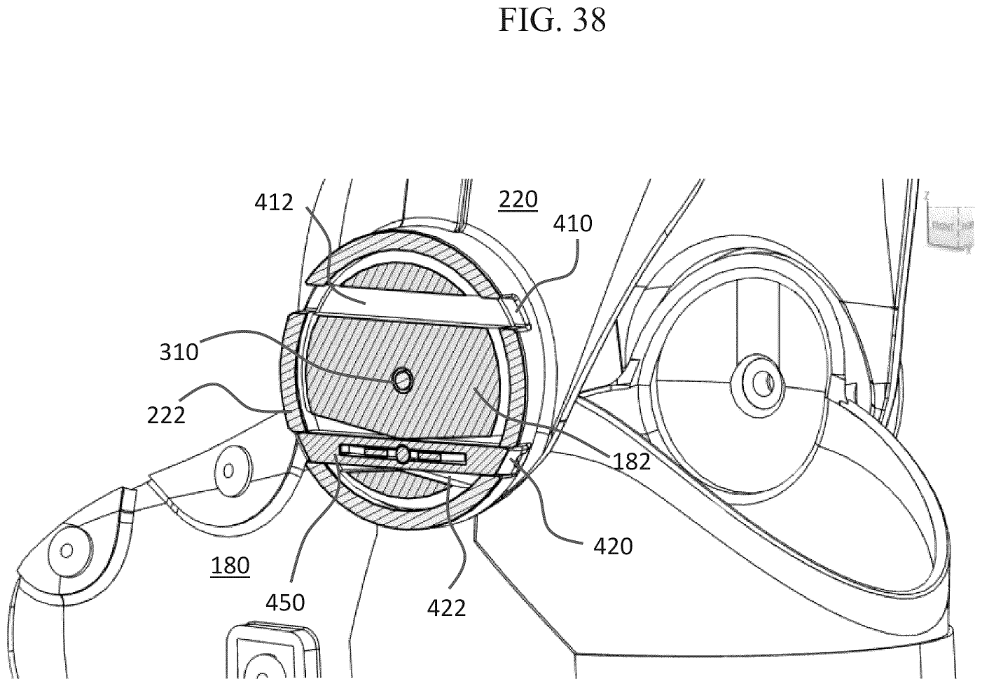

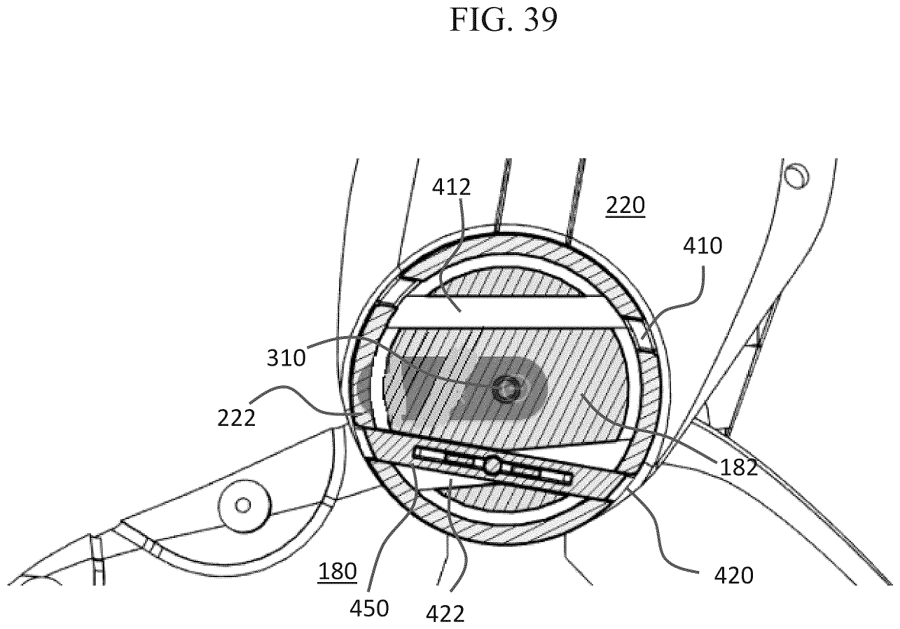

As seen in FIG. 37, the passageway 422 is a compound passageway as compared to the passageway 412. The oblique angles of the surfaces that establish passageway 422 are sized and dimensioned so that a limited amount of pivoting forward and backward can be accomplished. the pivoting is limited by the angle, and thus when the bar rotates with pivoting of the upright, when the bar hits the pertinent surface of the male medial joint portion 182 that establishes the passageway 422, further pivoting will be halted. FIG. 39 presents an exemplary scenario of use where rearward pivoting of the medial upright extension 220 is at a maximum because lock bar 450 has been rotated to hit the left upper surface of the passageway 422 and or the right lower surface of the passageway 442 as shown. Further rotation can thus not occur. The opposite would be the case if the upright was pivoted forward.

And note that the limited angle for forward pivoting can be different than the limited angle for rearward pivoting by adjusting the angles of the pertinent surfaces of the passageway. If the surfaces that are shown in FIG. 39 that contact the back bar were angled by a less amount than the opposite surfaces of the passageway, forward pivoting would be greater than rearward pivoting, and vice versa.

And to achieve the full range of forward and/or rearward pivoting, the lock bar(s) are not utilized in the joint. And this is just an exemplary embodiment—in some embodiments, the utilization of the lock bar may only limit pivoting in the forward direction or pivoting in the rearward direction relative to the full range of pivoting in those directions—for example, the lock bar might limit pivoting in the rearward direction to only ⅔rds of the total amount possible, and may not limit forward pivoting by any amount (except that which is limited by the overall structure without the lock bar).

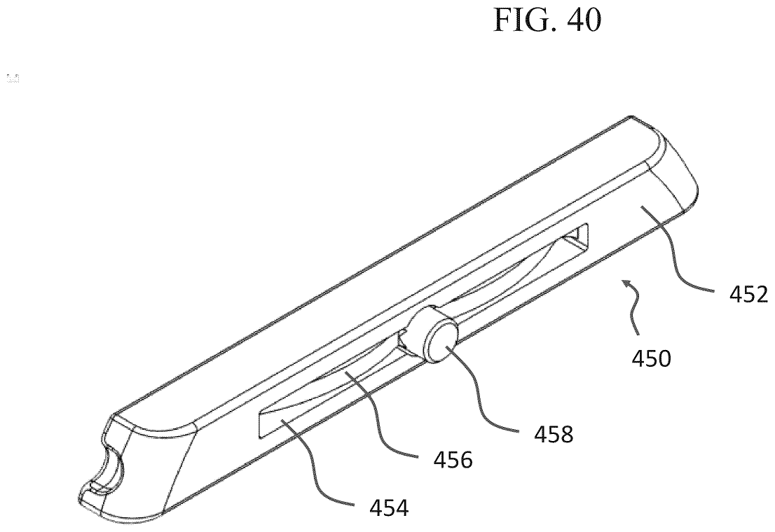

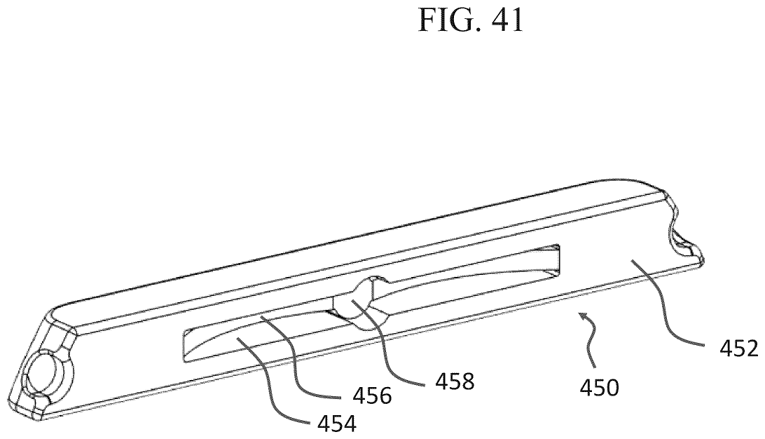

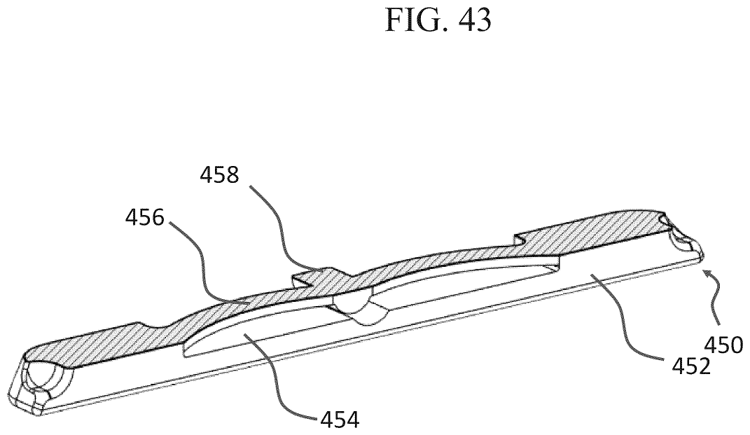

FIGS. 40 to 44 show various features associated with the lock bar 450. In an exemplary embodiment, the same design lock bar can be utilized for both the upper and the lower channels on either side. In an exemplary embodiment, the lock bar comprises a spring component 456 that is embedded in a polymer body 452 (the body can be formed/molded/casted about the spring). The spring component supports a male portion 458 that sat fits into the cylindrical bores/holes 1641 and 1642 in the female medial joint portion 222 (and the female lateral joint portion 202 when use therewith). The spring is a leaf spring analogous to the leaf springs of a truck or a car (as opposed to a coil spring, but a coil spring or ball (cylinder) detent could be used in some embodiments as the male portion 458 interfaces with the surface of the female joint portion that faces the male joint portion in the direction parallel to the axis of rotation, the male portion is pressed in word and thus the spring flexes inward into slot 454 in the body 452. When the male portion gets to the cylindrical bore. The male portion will pop up into the hole owing to the spring forces and because the male portion is in the bore, further longitudinal movement of the lock bar will be limited or otherwise prevented unless additional force in the longitudinal direction is applied. When the male portions 458 snap into the holes 1641, it will lock the lock bar is in place and keep them from moving in the longitudinal direction. The spring and the bodies and the male portions are sized and dimensioned so that upon a sufficient amount of pressing force in one direction or the other in the longitudinal direction, the spring force will be overcome and the locks can be moved out of the passageways so as to “unlock” the joint. (It is noted that we use “unlock” to also refer to the second stage where there is limited movement.) In an exemplary embodiment, the lock bar can be inserted and removed from the same side of the female joint portion or the lock bar can be inserted from one side and removed from the other side of the female joint portion. And in some embodiments, different lock bars can be utilized such that one lock bar will not fit into the passageway for the other lock bar. This can have utilitarian value with respect to providing a lock bar that is used to totally eliminate pivoting, and another lock bar that is configured to limit pivoting but not prevent pivoting. This can have utilitarian value with respect to not requiring the user to remember that it is the top passageway that is utilized for total elimination of pivoting, and vice versa. And note that while the top passage has been presented for the elimination of pivoting, the bottom passageway could be utilized for such. And note that in some embodiments, there is only a two mode approach, so one of the passageways might not be present and in an exemplary embodiment, one of the passageways can be closed off to prevent a user from utilizing one of the modes if such has utilitarian value.

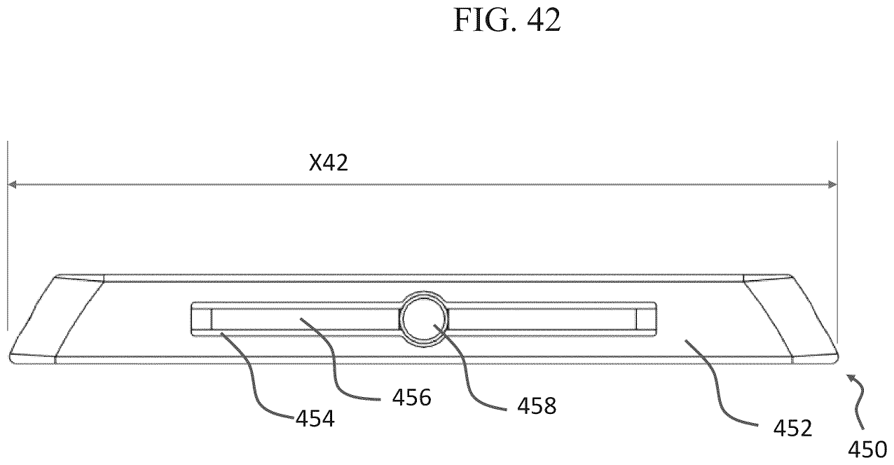

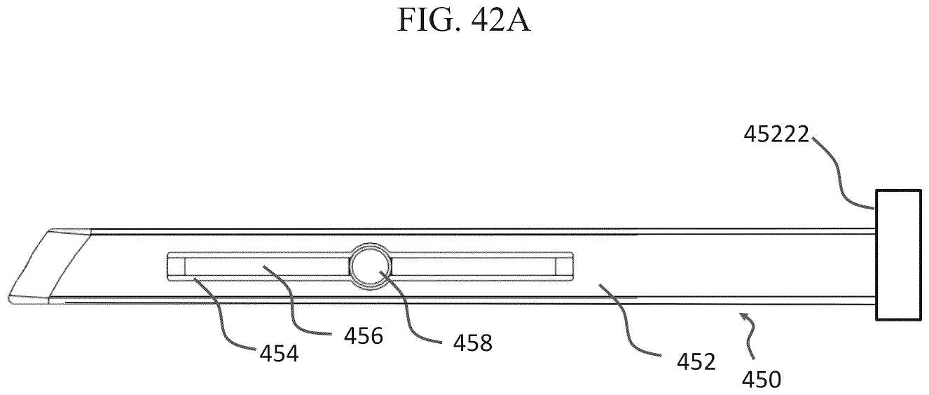

An exemplary length of the lock bar can be X42, which can be 1.5, 1.75, 2, 2.25, 2.5 or 3 inches or more or less or any value or range of values therebetween in 0.0001 inch increments. Consistent with the embodiments herein, the size and dimension of the lock bar can be scaled. A thickness of the lock bar can be 0.125, 0.1875, 0.25 or 0.3875 or more or less or any value or range of values therebetween in 0.0001 inch increments. A height of the lock bar can be 0.125, 0.1875, 0.25 or 0.3875 or more or less or any value or range of values therebetween in 0.0001 inch increments. Note that the lock bar need not be symmetric. Indeed, FIG. 42A shows that the bar extends further to the right to provide a clearance for a handle/finger grip 45222 to better facilitate insertion and/or removal of the bar from the brace.

The lock bar body (as opposed to the spring) can be made of the same material as the bulk of the uprights and/or the heel enclosure (bulk thereof). The male portion can be made of the same material or part of the spring material or a composite thereof. The male portion can be a separate component attached to the spring by welding or adhesion or screwing.

As noted above, the hinge assembly (pivot assembly) can be configured to provide different modes of use. In this regard, in some embodiments, the hinge is a lockable hinge that can be unlocked to enable movement and/or adjustment. In some exemplary embodiments, the hinge is set to be locked at a specific angle (90 degrees from the horizontal, for example). In some exemplary embodiments, the hinge is adjusted to enable free range of motion over a wide degree of angular movements relative to the sidewalls. By way of example only and not by way of limitation, the amount of angular movement could be, relative to the locked angle, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 degrees or any value or range of values therebetween in 0.1 degree increments dorsiflexion and/or plantar flexion (and the forward can be the same or different than the backwards angular movement).

In an exemplary embodiment, the hinge assembly is configured to provide only a limited range of motion, or more accurately, configured to permit only a limited range of motion (when the lock bar is present, for example). By way of example only and not by way limitation, limited range of motion can be a fraction of the aforementioned full range of motion. By way of example only and not by way limitation, the limited range of motion could be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32 or 32 degrees or any value or range of values therebetween in 0.1 degree increments less than and/or equal to those which corresponds of the movements noted above. By way of example only and not by way limitation, the limited range of motion could be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 degrees dorsiflexion and/or plantar flexion, or any value or range of values therebetween in 0.1° increments, and again, the range of motions can be different (e.g., the hinge can allow for 5° of dorsiflexion and 10 degrees of plantar flexion (from the locked angle)).

To be clear, a forward rotation limit could be greater than the reward rotation limit. For example, the forward rotation could be 1.5, 2, 2.5 or 3 times that of the rearward rotation (or visa-versa). It is noted that there might be no rearward rotation in some embodiments. Only forward rotation.

It is noted that some but not all embodiments of the lock bar/passageway arrangement allows a very limited amount of flexure/rotation even when the lock bar is in the top location. In this regard, for a torque applied to an upright equal to 10, 20, 30, 40 or 50 foot-pounds forward and then in the backward direction (or visa-versa), there will be less than and/or equal to 5, 4.5, 4, 3.5, 3, 2.5, 2, 1.5, 1, 0.5, 0.4, 0.3, 0.2 or 0.1 degrees or any value or range of values therebetween in 0.01 degree increments in total rotation (from the most forward location to the most rearward or visa-versa). That said, the aforementioned values can be considered.

In an exemplary embodiment, the brace is configured so that a torque applied to an upright equal to 10 foot-pounds forward and/or backward so that the stops are hit in the respective directions will have the upright at a first location (potentially for example 0.1 degrees off of the Z axis), and then an increase from that torque equal to 10, 15, 20, 25, 30, 35, 40, 45 or 50 foot-pounds (e.g. to a total of 60 foot-pounds) will result in no more than and/or equal to 3, 2.5, 2, 1.5, 1, 0.5, 0.4, 0.3, 0.2 or 0.1 degrees or any value or range of values therebetween in 0.01 degree increments in additional rotation (from the most forward location to the most rearward or visa-versa).

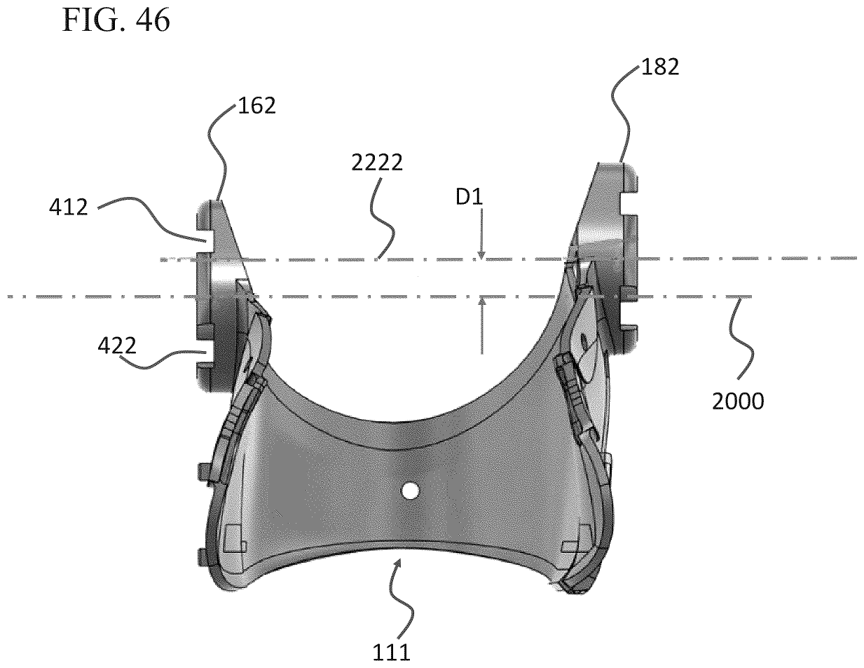

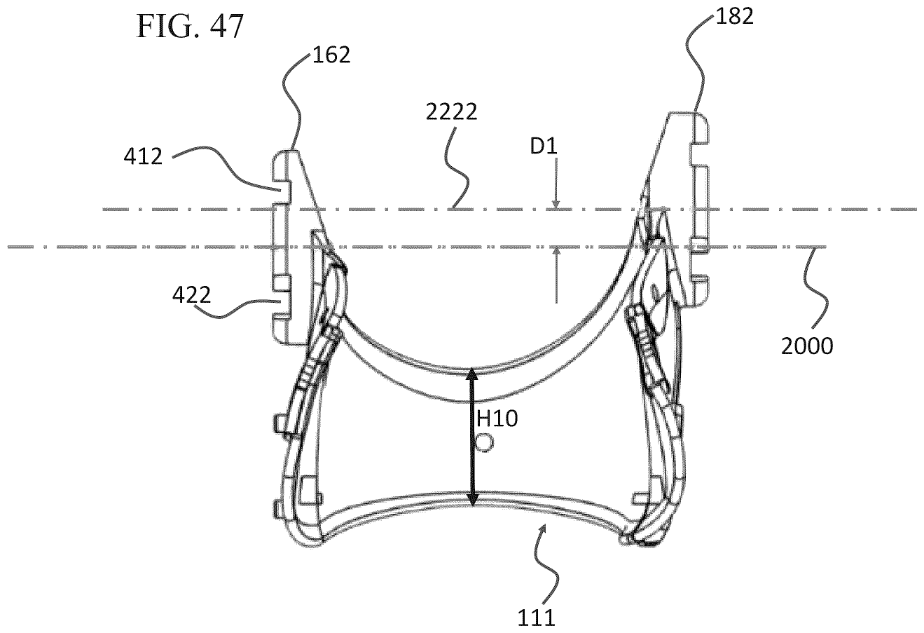

FIGS. 46 and 47 present a view looking down the X axis of the rigid heel enclosure 111 of the embodiment of FIG. 14.

FIG. 47 shows H10, which is a height of the rearmost portion of the rigid heel enclosure. In an exemplary embodiment, H10 can be less than and/or equal to and/or greater than 0.5, 0.75, 1, 1.25, 1.5, 1.75, 2, 2.25, 2.5, 2.75, 3, 3.25 or 3.5 inches or any value or range of values therebetween in 0.0001 inch increments (to achieve English units for example).

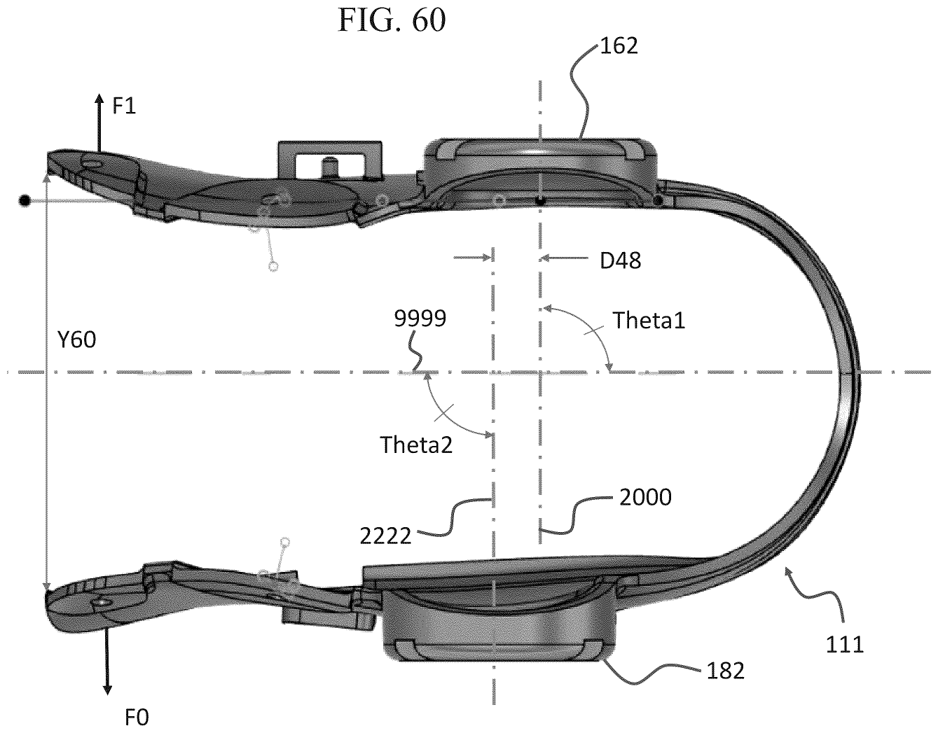

FIG. 60 shows a dimension Y60 corresponding to the distance between the most forward tips of the respective sidewalls in the relaxed state of the rigid heel enclosure. F1 represents a force applied away from the centerline at an angle of 90° there from in the XY plane, and F0 represents a holding force in the opposite direction. F0 and F1 can be located 0.25, 0.5, 0.75 or 1 inch from the ends or can be located at the ends of the sidewalls. In an exemplary embodiment with the straps unsecured (the bottom strap disconnected from the side and the top strap decoupled from each other), upon the application of a 0.1, 0.2, 0.3, 0.4, 0.5, 0.6, 0.7, 0.8, 0.9, 1, 1.1, 1.2, 1.3, 1.4, 1.5, 1.75, 2, 2.25, 2.5, 2.75 or 3 pound force for F1 at the noted locations, the dimension Y60 will increase from the relaxed state by less than and/or equal to and/or greater than 1, 2, 3, 4, 5, 6, 7, 8, 9, 10, 11, 12, 13, 14, 15, 16, 17, 18, 19, 20, 21, 22, 23, 24 or 25% or any value or range of values therebetween in 0.1 percent increments. Note that this can also be the case for the uprights. For purposes of textual economy, if F0 and F1 are taken from the ends of the uprights, and Y60 is measured from the comparable location (at the end), the changes in Y60 can be any one or more of the above (they need not be the same as that for the sidewalls—this is presented simply for purposes of textual economy).

Some exemplary embodiments will be described herein by way of example only and not by way limitation with reference to a device referred to in some instances as TayCo, TayCo Brace, TayCo External AFO, etc., and variations thereof. These constitute exemplary braces according to at least some exemplary embodiments. In some instances, the embodiments of the braces disclosed herein and/or variations thereof will be compared to other types of braces and/or other types of devices that provide some utilitarian value with respect to treatments of elements associated with the lower leg/ankle/foot. These are not embodiments of the braces disclosed herein, but are instead devices upon which the braces disclosed herein, such as the TayCo brace, improve upon, and are thus presented herein by way of example for comparison purposes and to further illustrate or otherwise provide details of the utilitarian features associated with the braces of the embodiments disclosed herein. By way of example only and not by way limitation, an exemplary CAM boot is disclosed below. This is not an embodiment of the brace disclosed herein, but instead is a device over which at least some exemplary embodiments disclosed herein provide utilitarian value.

Embodiments can include an all external ankle/hindfoot AFO for use in acutely injured football players, for example. The device can be a custom fitted device which can be dispensed for a wide variety of patients with various ankle/hind foot injuries and pathologies at the time of their initial presentation to the clinic. This custom fit, functional AFO can avoid, for example, the significant complications associated with using other ankle/hindfoot immobilization devices such as cam walkers, internal AFOs, and casts. All of these devices also have significant compliance and complications associated with them, and some embodiments can overcome at least one or more of these complications.

Embodiments include a Custom Fit TayCo External Ankle Brace (TayCo XAB) which can be a truly custom fit ankle foot orthosis which allows the patient to be immobilized as if they were in a traditional AFO, but without one or more of the associated complications of prior braces. The custom fit device is constructed of material which can be bent, molded, and trimmed right in the supplier's facility and/or at another location. Spring steel can be used to aid bending. The device has an option to limit range of motion and can also be converted to a free range of motion as per the utilitarian value of a given acutely injured patient.

By fitting external to the shoe/work boot, the Custom Fit TayCo External Ankle Brace permits the patient to wear his or her own shoe. This avoids one or more or all compliance issues associated with wearing clumsy or heavy walking boots and internal AFOs. Embodiments enable patients to wear their own shoes also avoids the induced limb length discrepancies and associated secondary knee, back, and hip pain and compliance issues with cam walkers and other internal AFOs. Thus, some embodiments enable users to wear their own footwear and/or can enable the avoidance of one or more compliance issues often associated with traditional AFOs, increasing patient compliance and patient comfort.

In some embodiments, the rigidity of the Custom Fit TayCo External Ankle Brace is equal to or superior to that of any AFO currently available in North America and/or the United States, and/or those approved by the US FDA as of Mar. 24, 2021. Embodiments can achieve this by, for example, the uniquely designed uprights and/or the manner by which the foot plate attaches to the shoe. By using circumferential forefoot compression as far distally as the metatarsal heads and custom uprights which extend to the calf, the brace can create a fundamental and/or intrinsic union with the shoe. This can enable the maintenance of the ankle foot correction, as, for example, prescribed by the physician. This can be the case even though in some embodiments, the foot plate is primarily surrounding, rather than simply underneath, the foot. In some embodiments, the device is sufficiently rigid so as to be designed to be in compliance with the DME MAC AFO LCD requirements.

As disclosed herein, some embodiments of the AFO can be a truly Custom Fit arrangement.

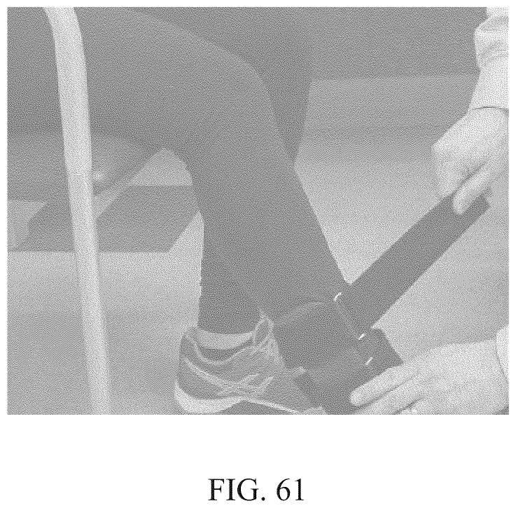

FIG. 61 presents a schematic of healthcare professional attaching a brace according to at least some exemplary embodiments to a patient. FIG. 62 presents a detailed view of the brace.

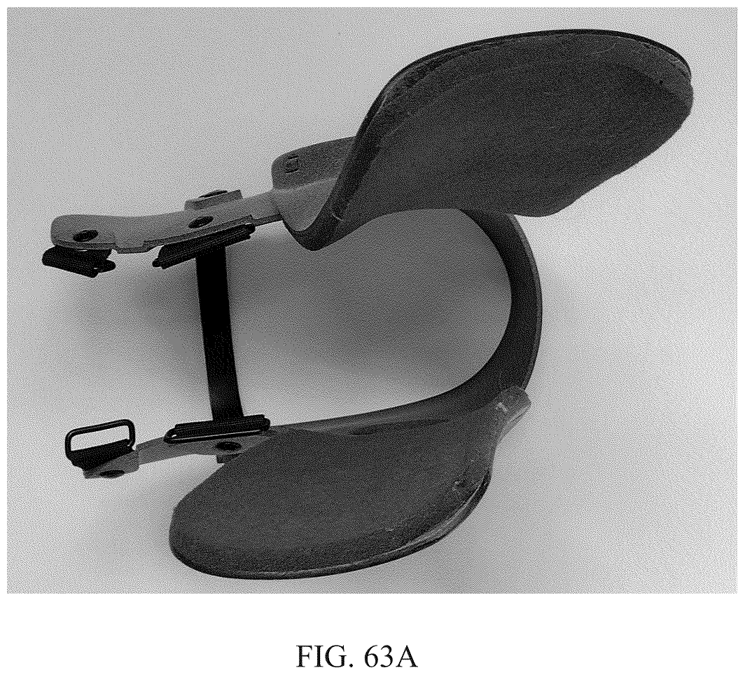

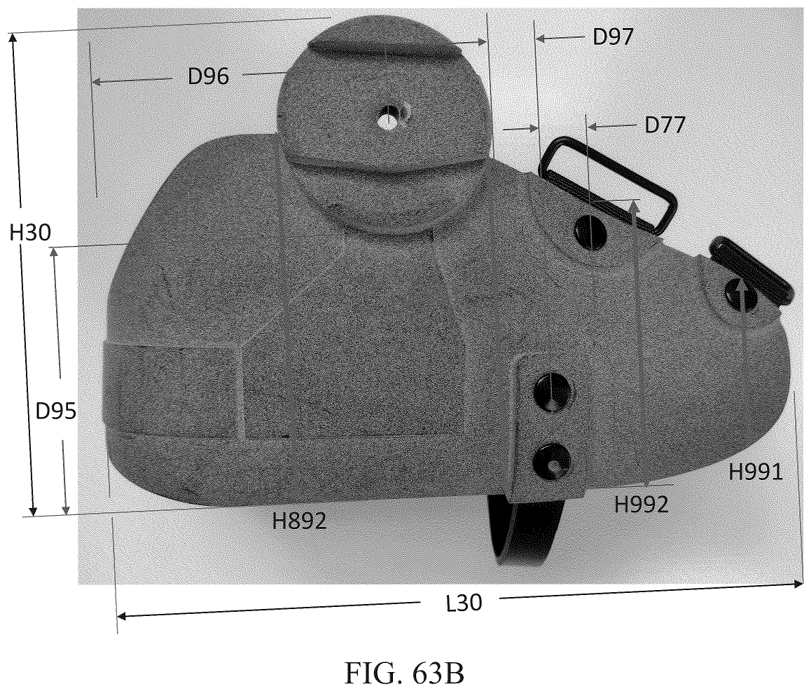

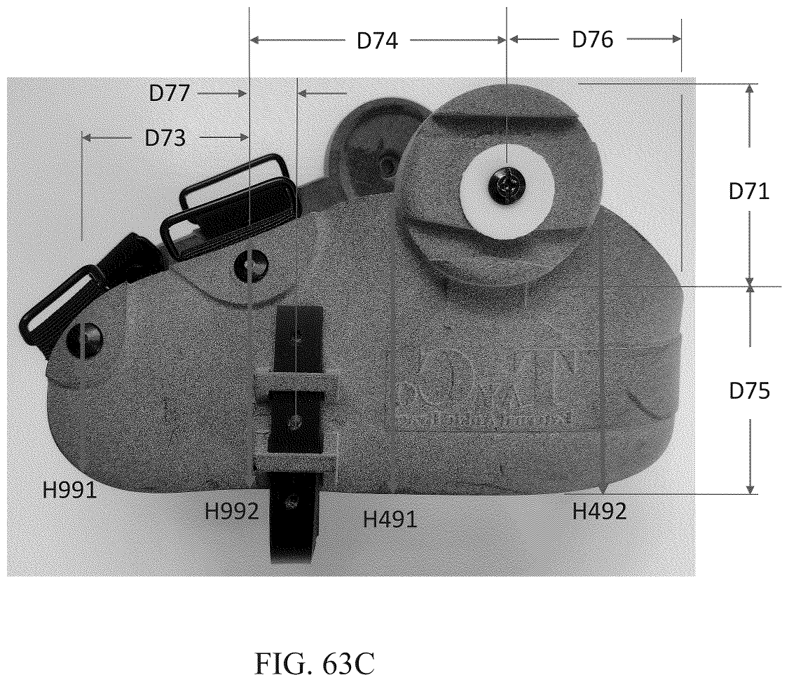

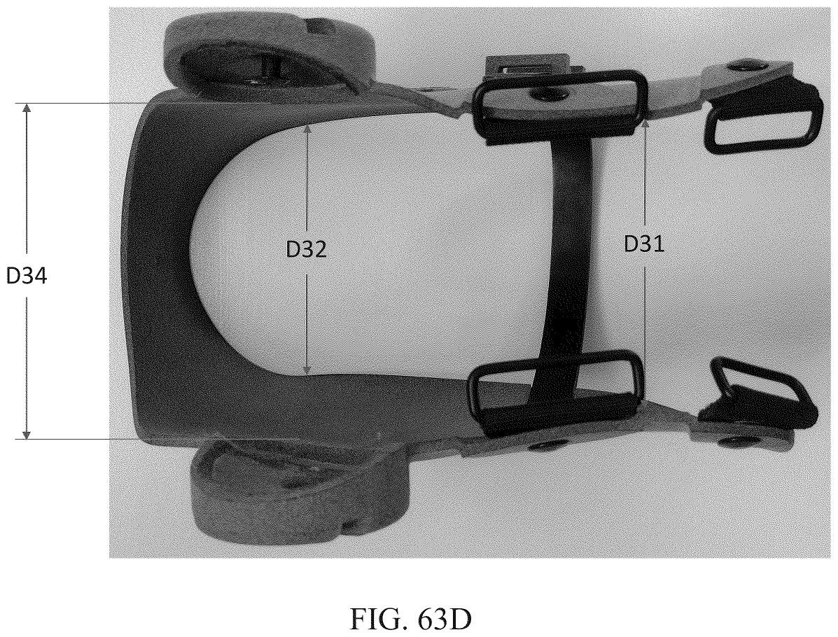

FIG. 63A shows a top view of the brace without the straps for clarity, and showing how the uprights can be articulated independent of each other (the hinges can be independently set/adjusted). FIGS. 63B and 63C show side views of the lower section with the uprights removed. FIG. 63D shows a top view of the lower section with the uprights removed. FIG. 63E shows an end view of the lower section with the uprights removed. FIG. 63F shows the uprights free of the lower section from the outside. FIG. 63G shows the uprights from the inside, with the lock members in the upright for the medial upright and not in the lateral upright. FIG. 63H shows the uprights when viewed from the front.

FIGS. 62-63H are figures shown an actual product and are to scale and all features can be scaled off of what is shown. Also, all values are the relaxed, at-rest unrestrained values (unencumbered by straps for example). Values for these will be different than comparable values disclosed above, and in other instances may be the same. L30 can be less than greater than or equal to 10, 11, 12, 13, 14, 15, 16, 17, 18, 19, 20, 21, 22, 23, 24, 25, 26, 27, 28, 29 or 30 cm or any value or range of values therebetween in 1 mm increments. H30 can be less than greater than or equal to 7, 8, 9, 10, 11, 12, 13, 14, 15, 16, 17, 18, 19 or cm or any value or range of values therebetween in 1 mm increments. The dimensions of the product shown are H30 equals 13 cm and L30 equals 21 cm. And thus because the values can be scaled, for example, if L30 is 27.3 cm, then H30 can be 27.3/21 times 13 cm, etc.

D31 can be less than greater than or equal to 4, 5, 6, 7, 8, 9, 10, 11, 12, 13, 14 or 15 cm or any value or range of values therebetween in 1 mm increments. D32 can be less than greater than or equal to 5, 6, 7, 8, 9, 10, 11, 12, 13, 14, 15 or 16 cm or any value or range of values therebetween in 1 mm increments. D34 can be less than greater than or equal to 6, 7, 8, 9, 10, 11, 12, 13, 14 or 15 cm or any value or range of values therebetween in 1 mm increments. The values of the product shown are D34=9.5 cm D32=9.5 cm D31=7.5 cm.

D41 can be less than greater than or equal to 2, 2.5, 3, 3.5, 4, 4.5, 5, 5.5, 6, 6.5 or 7 cm or any value or range of values therebetween in 1 mm increments. D40 can be less than greater than or equal to 4, 5, 6, 7, 8, 9, 10, 11, 12, 13, 14 or 15 cm or any value or range of values therebetween in 1 mm increments. The values of the product shown are D40=8 cm and D41=4 cm.

D51 can be less than greater than or equal to 4, 4.5, 5, 5.5, 6, 6.5, 7, 7.5, 8, 8.5, or 9 cm or any value or range of values therebetween in 1 mm increments. D52 can be less than greater than or equal to 5, 5.5, 6, 6.5, 7, 7.5, 8, 8.5, 9, 9.5, 10, 10.5, 11, 11.5, 12, 12.5, 13, 13.5 or 14 cm or any value or range of values therebetween in 1 mm increments. L55 can be less than greater than or equal to 14, 15, 16, 17, 18, 19, 20, 21, 22, 23, 24, 25, 26, 27, 28, 29, 30, 31 or 32 cm or any value or range of values therebetween in 1 mm increments. The values of the product shown are L55=23.5 cm, D52=9.2 cm, D51=6.4 cm.

D61 can be less than greater than or equal to 3, 3.5, 4, 4.5, 5, 5.5, 6, 6.5, 7 or 7.5 cm or any value or range of values therebetween in 1 mm increments. D71 can be less than greater than or equal to 3.5, 4, 4.5, 5, 5.5, 6, 6.5, 7, 7.5, 8 or 8.5 cm or any value or range of values therebetween in 1 mm increments.

FIG. 63C shows dimensions H491 and H492, which are presented above. Also shown are dimensions H991 and H992, which are vertical and taken through the center of the rivets for the top straps (the center of attachment of the top straps). H991 can be less than greater than or equal to 3, 3.5, 4, 4.5, 5, 5.5, 6, 6.5, 7, 7.5, or 8 cm or any value or range of values therebetween in 1 mm increments. H992 can be less than greater than or equal to 5, 5.5, 6, 6.5, 7, 7.5, 8, 8.5, 9, 9.5, 10, 10.5, 11, 11.5, 12, 12.5, 13, 13.5, 14, 14.5 or 15 cm or any value or range of values therebetween in 1 mm increments. With respect to the product shown, H991 is 5 cm. H992 is 8 cm.

D73 can be less than greater than or equal to 3, 3.5, 4, 4.5, 5, 5.5, 6, 6.5, 7, 7.5, or 8 cm or any value or range of values therebetween in 1 mm increments. D74 can be less than greater than or equal to 5, 5.5, 6, 6.5, 7, 7.5, 8, 8.5, 9, 9.5, 10, 10.5, 11, 11.5 or 12 cm or any value or range of values therebetween in 1 mm increments. D73 is 5 cm in the product shown, and D74 is 7 cm in the product shown. D75 can be less than greater than or equal to 4, 4.5, 5, 5.5, 6, 6.5, 7, 7.5, 8, 8.5, 9, 9.5 or 10 cm or any value or range of values therebetween in 1 mm increments. D75 is 6 cm in the product shown. Note that the screw in the joint portion is centered with respect to D71. D76 is 7.5 cm in the product shown, and can be less than greater than or equal to 5, 5.5, 6, 6.5, 7, 7.5, 8, 8.5, 9, 9.5, 10, 10.5, 11, 11.5 or 12 cm or any value or range of values therebetween in 1 mm increments. D77 is 1.5 cm in the product shown, and can be less than greater than or equal to 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4 or 2.5 cm or any value or range of values therebetween in 1 mm increments.

D95 can be less than greater than or equal to 4.5, 5, 5.5, 6, 6.5, 7, 7.5, 8, 8.5, 9, 9.5, 10, 10.5 or 11 cm or any value or range of values therebetween in 1 mm increments. D95 is 7 cm in the product shown. Note that the hole in the joint portion is centered with respect to the circular joint. D96 is 9.5 cm in the product shown, and can be less than greater than or equal to 6, 6.5, 7, 7.5, 8, 8.5, 9, 9.5, 10, 10.5, 11, 11.5, 12, 12.5, 13 or 13.5 cm or any value or range of values therebetween in 1 mm increments. D97 is 1.5 cm in the product shown, and can be less than greater than or equal to 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4 or 2.5 cm or any value or range of values therebetween in 1 mm increments.

H892 is 9.0 cm in the product shown and can be less than and/or equal to and/or greater than 5, 5.5, 6, 6.5, 7, 7.5, 8, 8.5, 9, 9.5, 10, 10.5, 11, 11.5, 12, 12.5, 13, 13.5, 14, 14.5 or 15 cm or any value or range of values therebetween in 1 mm increments. Again, this is to scale, so the other heights of the sidewall can be scaled. H891 is 9.0 cm in the product shown and can be less than and/or equal to and/or greater than 5, 5.5, 6, 6.5, 7, 7.5, 8, 8.5, 9, 9.5, 10, 10.5, 11, 11.5, 12, 12.5, 13, 13.5, 14, 14.5 or 15 cm or any value or range of values therebetween in 1 mm increments.

H892 is 10.5 cm in the product shown and can be less than and/or equal to and/or greater than 5, 5.5, 6, 6.5, 7, 7.5, 8, 8.5, 9, 9.5, 10, 10.5, 11, 11.5, 12, 12.5, 13, 13.5, 14, 14.5 or 15 cm or any value or range of values therebetween in 1 mm increments. Again, this is to scale, so the other heights of the sidewall can be scaled. H891 is 9.0 cm in the product shown and can be less than and/or equal to and/or greater than 5, 5.5, 6, 6.5, 7, 7.5, 8, 8.5, 9, 9.5, 10, 10.5, 11, 11.5, 12, 12.5, 13, 13.5, 14, 14.5 or 15 cm or any value or range of values therebetween in 1 mm increments.

All dimensions herein can be exact and/or plus or minus 0.010, 0.02, 0.03, 0.04, 0.05, 0.06, 0.07, 0.08, 0.09 or 0.1 inches or any value or range of values therebetween in 0.001 inches. Again, all values are at rest/unrestrained/without load.

All dimensions can be scaled from the figures, and disclosures include taking those dimensions and adding plus and/or minus 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35, 36, 37, 38, 39, 40, 41, 42, 43, 44 or 45% or any value or range of values therebetween in 0.1% increments, providing that the art enables such. (E.g., D77 can be increased by 20% of 1.5 cm (thus resulting in 1.8 cm), or 5% of 1.5 cm, or decreased by 17% of 1.5 cm, etc.).

In an exemplary embodiment, the lower portion of the brace is configured so that when one sidewall is supported at the very distal portion thereof or within 1 cm of the distal portion by a line/point reaction device 110011, as shown in FIG. 96, a force F110 can be applied to an opposite sidewall at the most distal portion thereof or within 1 cm of the distal portion by a point force or a line force to compress the two sidewalls together so that the sidewalls and/or components carried thereby that are proximate the sidewalls first contact each other at the force F110, wherein F110 can be less than, greater than and/or equal to 0.15, 0.2, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65 or 1.7 pounds or any value or range of values therebetween in 0.01 pound increments. (Note that the sidewalls are free to move—straps are removed, and the uprights are unrestrained—all is unrestrained, the only resistance is the material of the lower portion.

In an exemplary embodiment, the brace is configured so that when one upright is supported at the very distal portion thereof or within 1 cm of the distal portion by a line/point reaction device 110011, as shown in FIG. 111, a force F111 can be applied to an opposite sidewall at the most distal portion thereof or within 1 cm of the distal portion by a point force or a line force to compress the two uprights together so that the uprights and/or components carried thereby (here, the foam) that are proximate the sidewalls first contact each other at the force F111, wherein F 111 can be less than, greater than and/or equal to 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, or 2.3 pounds or any value or range of values therebetween in 0.01 pound increments. (Note that the uprights are free to move—straps are removed, and the uprights are unrestrained—all is unrestrained, the only resistance is the material of the lower portion which carries the uprights.

In an exemplary embodiment, there are medial and lateral uprights made of orthotic grade plastic with optionally spring steel insert that aids in the custom fit of the brace. The footplates (bottom portion) can be orthotic grade plastic that wraps tightly around the shoe with an under strap on the bottom and Velcro on the top. Foam padding can be on the inside of the uprights. The uprights can provide for soft tissue interface. In some embodiments, the uprights extend to the calf and/or the footplates extend to metatarsal heads of a given human factors engineering person male or female (e.g., a 5, 10, 15, 20, 25, 30, 35, 40, 45, 50, 55, 60, 65, 70, 75, 80, 85, 90 or 95 or 100 percentile or any value or range of values therebetween in 1% increments human factors engineering (HFE) male or female born in the United States in 1950, 1960, 1970, 1980, 1990, 2000, 2010, 2011, 2012, 2013, 2014, 2015, 2016, 2017, 2018, 2019, 2020, 2021 or 2022 as of this filing).

In some embodiments, Velcro straps wrap around and are attached to the uprights, and are adjustable to adjustably secure the uprights to the lower leg. Optional locking bars can be used to lock the uprights and/or enable the uprights to rotate. Also, a variable hinge can be present that permits rotation on a limited amount.

Adjustable urethane straps can be present on the top of the footplates (two are shown, but one or three or four can be used.

The orthotic grade plastic of at least some of the components can be bent, molded and/or trimmed to achieve a custom fit.

As seen in FIG. 64, in some exemplary embodiments, the brace can be custom fit to an individual (as opposed to custom fabricated—more on this below). It is noted that some embodiments do not necessarily require the ability for custom fitting. FIG. 62 also presents exemplary features that may be present in at least some exemplary embodiments, while it is noted that in some embodiments, these features may not be present or otherwise may be modified.

FIG. 64 presents a schematic coupled with annotations that provide an exemplary method according to an exemplary fitting method according to some exemplary embodiments. Here, it can be seen that there is a process that can be followed in some exemplary scenarios of use of the brace which can have utilitarian value with respect to fitting the brace on to a human relative to another human. In this regard, by way of example only and not by way limitation, embodiments include methods of fitting and/or utilizing at least some of the exemplary embodiments of at least some of the exemplary braces disclose herein and/or variations thereof. It is noted that any method action disclosed herein can be executed with other types of braces that are not necessarily disclosed herein providing that the art enable such. And as is detailed above, any one or more of the features of the method(s), such as for example, one of the steps or substeps of the method of FIG. 65 can be skipped or augmented or modified or otherwise combined with another method if there is utilitarian value in doing so.

FIG. 65 presents a schematic of some exemplary options of use that can be enabled utilizing some exemplary hinges according to some exemplary embodiments. In this regard, it can be seen that the hinge can be set to provide a free range of motion and/or a limited range of motion and/or can immobilize movement of the top of the brace relative to the bottom of the brace. In at least some exemplary embodiments, the user of the brace can “control” the various options of hinge movement. In this regard, the user can on his or her own change the range of motions that are available or otherwise that can be provided by the brace to suit his or her or desires or otherwise needs at that time and/or in the future. That said, in an alternate embodiment, the hinge is configured so that it requires a healthcare professional or at least someone with more detailed mechanical knowledge to change the hinge options. This can have utilitarian value with respect to ensuring that a patient maintains a given therapy regime desired by healthcare professional. For example, there could be utilitarian value in the recipients of the brace having a brace that is in the immobilize state for at least a week.

FIG. 65 thus shows a process that can be followed in some exemplary scenarios of use of the brace which can have utilitarian value with respect to setting different modes of the brace. FIG. 65 shows hinge options, and how the hinge can be adjusted/how the brace can be adjusted via adjustment of the configurations of the locking bar and hinge allowing for multiple treatment options.

More particularly, FIG. 65A shows the configuration where there is free motion of the uprights relative to the footplates. Here, the locking bar is removed to allow for full dorsiflexion/plantar flexion motion of the ankle joint. This provides for a free motion configuration of the brace. FIG. 65B shows a limited range of motion configuration of the brace where the locking bar is moved to the lower slot and allows for 5 degrees of dorsiflexion and 10 degrees of plantar flexion. FIG. 65C shows the immobilized configuration, where the locking bar prevents any dorsiflexion/plantar flexion of the ankle joint.

FIG. 66 shows an exemplary brace applied to a work boot. FIGS. 67 and 69 shows the application of a brace to a walking shoe/home shoe/sneaker while walking. FIG. 68 shows a brace for the opposite leg relative to that of FIG. 67. FIG. 74 shows the use of the brace while jogging.

FIG. 76 shows use of the brace while walking down a set of concrete steps.

Methods thus include adjusting the configuration of the brace between the three modes detailed above and/or setting an adjustment based on a treatment regime.

By way of example only and not by way limitation, embodiments include methods of fitting and/or utilizing at least some of the exemplary embodiments of at least some of the exemplary braces disclose herein and/or variations thereof. It is noted that any method action disclosed herein can be executed with other types of braces that are not necessarily disclosed herein providing that the art enable such. And as is detailed above, any one or more of the features of the method(s), such as for example, one of the steps or substeps of the method of FIG. 65 can be skipped or augmented or modified or otherwise combined with another method if there is utilitarian value in doing so.

The healthcare professionals could desire that the recipient not adjust the hinge option to provide the various motions. Thus, by providing a more complicated locking arrangement, the user may not be able to adjust the hinge options. This can be particularly utilitarian with respect to children who may not be able to appreciate the utilitarian value of maintaining the brace in a given state, where the children would be less able to adjust the hinge options.

FIG. 66 presents some exemplary options they can be utilized in some exemplary embodiments.

In some embodiments, the teachings herein can be related to/have the following HCPCS Codes: L1971, L2820 and/or L2200 X2. For example, these can be related to an ankle foot orthosis, plastic or other material with ankle joint, prefabricated, includes fitting and/adjustment. Also by way of example, these can be related to addition to lower extremity orthosis, soft interface for molded plastic, below knee section. (This can be achieved via the foam padding on the uprights and/or ankle joint). Also, the teachings herein can be related to the addition to lower extremity, limited ankle motion, each joint. (This can be achieved by the hinge on both joints that can limit range of motions.)

Embodiments can be worn so that the uprights are on the outside of a pants leg (lower pants leg) or inside pants (and outside a sock, for example). The Footplate can wrap around the shoe providing compression and corrective forces to the foot/ankle/lower leg. The footplate and shoe assembly work together to control pronation and supination.

In some embodiments, the brace is designed to be custom fit for each individual patient, and thus there are methods of doing so. The brace can work in harmony with the patient's footwear and/or orthotics to control pronation and/or supination.

FIG. 75 is a pictorial depicting the brace according to at least some exemplary embodiments in use on a left leg of respective Homo sapiens wearing sneakers, and FIG. 70 depicts application of the brace.

FIG. 71 provides some details associated with observations made by users regarding the brace(s) according to some exemplary embodiments. In this regard, it is noted that at least some exemplary embodiments of the braces detailed herein have one or more of the features and/or utilitarian aspects indicated by one or more of the people who have used the brace and/or have knowledge associated with the brace(s) as detailed herein, such as is detailed in FIG. 71. Moreover, FIG. 71 presents an additional exemplary method according to a method that provides for utilitarian fitting of the brace according to some exemplary embodiments. The method algorithms depicted in the figures present exemplary methods of fitting (note that sometimes herein, the brace is referred to as the TayCo, and this is a trademarked name, and refers to the specific embodiment that is disclosed with reference thereto. That is the method algorithms provide additional details of an exemplary method of custom fitting at least some exemplary embodiments of the braces detailed herein, and also presents an exemplary method of practice according to some exemplary embodiments.

FIGS. 77 and 80 provide exemplary methods of sizing/fitting/applying the brace.

FIG. 78 presents an exemplary method of modifying the hinges according to an exemplary embodiment. FIG. 79 presents an exemplary method of using the brace with athletic tape.

Embodiments of the braces detailed herein can be utilized with standard walking and/or jogging shoes and/or workboots. Some embodiments can be utilized with standard dress shoes such as Oxford dress shoes. Embodiments can be utilized for a wide range of injuries, including, for example, ankle sprains, stable fractures, and/or operative fractures.

Embodiments enable a recipient of the brace to walk and permit the recipient to return to normal daily activities. Embodiments of the brace can weigh less than and/or equal to 3.5, 3, 2.5, 2, 1.9, 1.8, 1.7, 1.6, 1.5, 1.4, 1.3, 1.2, 1.1, 1.0, 0.9, 0.8, 0.7, 0.6 or 0.5 pounds or less or any value or range of values therebetween in 0.05 pound increments (1.15, 0.95, 0.8 to 1.25 pounds, etc.). In an embodiment, the footplates/bottom portion of the brace (sidewalls and connecting wall at the back) increase the volume of the shoe area by no more than 5, 6, 7, 8, 9, 10, 11, 12, 13, 14, 15, 16, 17, 18, 19, 20, 21, 22, 23, 24, 25, 26, 27, 28, 29 or 30% or any value or range of values therebetween in 0.1% increments when worn over the shoe.

FIGS. 72 and 73 presents an exemplary evaluation matrix that compares a control walking boot/non embodiment of the teachings herein to the brace according to at least some exemplary embodiments, identified as the “acute Tayco external angle brace.” As seen, at least some exemplary embodiments of the ankle braces detailed herein can provide superior utilitarian features as compared to control device walking boots.

FIG. 74 presents some exemplary features according to some of the exemplary embodiments that can, in at least some exemplary instances, provide utilitarian value over and beyond or otherwise remedy the perceived problems of the control devices articulated above.

Embodiments can provide stability for athletes, and can be worn while playing soccer, football, golf, baseball, including doing so professionally in games. Embodiments can be used to treat acute bone and/or soft tissue injuries. The braces herein can serve as an alternative/substitute to walking boots. The braces herein can be used to treat chronic ankle injuries and to treat balance and stability disorders.

Embodiments can be used to immediately treat injuries, and enable the injured to return to the activity to which he or she was engaging in shortly after donning the brace (e.g., within 5, 4, 3, 2 or 1 minutes).

Moreover, embodiments can be used as a prophylactic against ankle injury.

Embodiments can utilize the brace to provide stability without limiting function to patients recovering from acute ankle injuries. The brace can fit over the user's existing footwear, and thus the user utilizes the same footwear as she or he utilized prior to adopting the brace, and as noted above, can utilize the exact footwear that the patient was utilizing her otherwise the recipient was utilizing at the time of the injury that necessitated were otherwise induced the adoption of the utilization of the brace. The brace can provide comfort and/or protection against further soft tissue damage. The brace accomplishes this by restricting pronation and supination of the ankle with options for mobilization and range of motion for plantarflexion and dorsiflexion.

The brace can be an equivalent of treatment with a cast and/or a walking boot. Embodiments can decrease the relative amount of back, hip and/or knee pain compared with that which results from utilizing a walking boot. The braces detailed herein can provide for a device that is compatible with a custom orthotic, this is compared to a walking boot for example. This can lead to improved patient compliance as well as improved outcomes.

As compared to comparative ankle foot orthoses (AFOs) and braces, the embodiments herein provide an external stability AFO for functional ankle treatment. Embodiments of the braces herein can provide excellent stability while allowing patients to comfortably use their own jogging shoes and/or work boots. Embodiments thus include jogging and/or running with the brace. With the TayCo brace fitting over the shoe, such can, in some embodiments, reduce and/or eliminate the overcrowding of the brace in the shoe (which can, in some embodiments at least, allow for less restrictive shoe options) and/or reduced pressure points that cause irritation due to bony deformities.

Embodiments of the External Ankle Brace can also provide a viable lightweight alternative (typically 12 ounces) to the control cast boot (which can range from two to four pounds). In contrast to cast boots can cause leg length discrepancies that can lead to secondary back, hip and knee problems, embodiments of the brace herein avoid and/or reduce the occurrence of such. The TayCo brace allows a “much quicker functional recovery” for work, shopping, playing, etc., relative to the control cast boot.

Some embodiments explicitly exclude the use of the TayCo External Ankle Brace for conditions such as spasticity, severe dropfoot and/or severe ankle deformity

D exemplary braces herein can provide substantial benefits over normal walking boots. By way of example only and not by way of limitation, consider the scenario of a 75-year-old who suffers a lateral malleolar fracture, and has ORIF surgery. This patient could have a posterior splint for two weeks, would be in a walking boot for four weeks (a NWB), and could be in a walking boot WBAT for 4 weeks. The patient could have limited function after 10 weeks and the patient could be deconditioned after 10 weeks. It is entirely possible in the scenario that ADLs could not be performed, and the patient would be admitted to the nursing home for three months of physical therapy. Conversely, utilizing the braces according to the teachings herein, there would be the scenario the posterior splint for two weeks, but utilizing the fixed brace, in an NWB scenario, the patient will be allowed to ambulate during a four-week period after the posterior splint is removed. Then, the brace could be converted to a ROM/WBAT, with gradually improved ADLS for 4 weeks. After 10 weeks the patient could be fully independent and can perform ADLs. Thus, the patient can return to ADLs at least 2, 2.5, 3, 3.5 or 4 times faster, and there is a reduction in physical therapy, such as by 3 months.

Consider another scenario where a 40-year-old weekend athlete suffers a grade 2 ankle sprain. Atypical scenario treatment would be the provision of a walking boot, with PWB to FWB over three weeks, with limited ADLs. This person would also have physical therapy for 3 weeks. Conversely, with the braces herein, a fixed brace would be used for 1 week (WBAT), and then the brace would be converted to ROM-FWB for 1 week. Then, full ADLs would be available, and physical therapy might be given for 2 weeks. Again, we see the return to ADLs faster (see the just mentioned timeframes above), and there is a reduction in PT of 1 week).

As noted above, embodiments can be applied to workboots/returning a worker to work status in a quick manner/protecting a worker while working from further injury. Embodiments can return a worker to normal daily activities and reduce lost workdays by at least 50, 55, 60, 65, 70, 75, 80, 85 or 90% compared to walking boots. As with the embodiments above, the braces herein can be utilized for treatment of acute bone and soft tissue injuries. Braces can fit workboots and/or walking and/or jogging shoes. The braces herein can be utilized to treat chronic ankle injuries and address balance and/or stability diagnoses. Below is an exemplary competitive matrix comparing a walking boot to the acute take of external brace according to at least some of the teachings detailed herein.

| | | | | | | OWN | | MINIMIZE | LIMB | FIXED |
|---|---|---|---|---|---|---|---|---|---|---|
| | SUGGESTED | | STA- | | DAILY | FOOT- | ORDER- | DECON- | LENGTH | & ROM |
| PRODUCT | CODES | COMFORT | BILITY | WEIGHT | FUNCTION | WEAR | ING | DITIONING | IMBALANCE | CAPABILITY |
| WALKING | L4386 or | LOW | HIGH | HEAVY | LOW | NO | EASY | NO | YES | NO |
| BOOT | L4370 or | | | (2-4 lbs.) | | | | | | |
| | L4360 or | | | | | | | | | |
| | L4361 or | | | | | | | | | |
| | L4361 | | | | | | | | | |
| ACUTE | L1971 and | HIGH | HIGH | LIGHT | HIGH | YES | EASY | YES | NO | YES |
| TAYCO | L2820 | | | (~14 oz.) | | | | | | |
| EXTERNAL | | | | | | | | | | |
| ANKLE | | | | | | | | | | |
| BRACE | | | | | | | | | | |

An exemplary treatment scenario can include a 38-year-old employee with moderate to severe ankle sprain. In a normal scenario, this employee would be given a walking boot, with no return to work for four weeks, and this walking boot would be open toe. The patient will be unable to return to work after four weeks due to lower back pain caused by limits length inequality from the walking boot. Conversely, with a brace according to the teachings herein, the employee would utilize the fixed brace configuration is detailed herein, and would return for limited duty work for one week. After which, the braces converted to a ROM brace and work with no restrictions for 3 weeks using the brace. thus, we see a return to limited duty of work for weeks faster than that which would be the case with respect to a walking boot. We see the employee working with no restrictions, three weeks faster than the scenario with the walking boot. This results in a reduction of lost workdays by 75%.

Consider another scenario where a 46-year-old employee with a bimalleolar fracture, who has ORIF surgery. Here, this person would have a posterior splint for two weeks, and would be NWB for 2 weeks. The person would be also put in a cast for the next four weeks and also be NWB. Following this, the patient would be given a walking boot, which would transition him from PWB to FWB, along with physical therapy, and this person would not return to work for another four weeks, along with having an open toe walking boot. The walking boot would then be utilized for another four weeks, in an FWB scenario, and there still is no return to work, while still having the open toe. Finally, the person would return to work with no restrictions at 16 weeks after the surgery. Conversely, this patient would have a posterior splint for the same amount of time in the same scenario, but then would be given the fixed brace according to the teachings detailed herein, and would be NWB for four weeks. Then, the brace would be converted to ROM, and the patient would go from PWB to FWB, and return to limited duty work for four weeks. After that, the patient would return to work with no restrictions at 10 weeks. Thus, we see a return to limited duty work 10 weeks faster with the brace according to the teachings herein, we see work with no restrictions, six-week faster with the brace according to the teachings detailed herein, and the reduction of lost workdays by 62.5%.

Thus, embodiments can include a reduction of lost workdays by utilizing the braces compared to the walking boot by at least 50, 55, 60, 65, 70, 75, 80, 85 or 90%.

Embodiments also enable otherwise provide a closed toe solution, because the worker can utilize his or her normal workboot or work shoe. This as compared to the walking boot, which is an open toe solution, and even if there is some form of closure, does not amount to the closed toe protection given by a standard workboots. Certainly, the walking boot does not provide for a steel tipped toe application, which can be achieved utilizing a workboot with the brace according to the teachings detailed herein.

It is explicitly noted that at least some exemplary methods of practice detailed herein include method actions that relate to the improvement of a function of a medical device, such as for example, the brace disclosed herein, and/or are directed towards the treatment of an ailment or otherwise malady afflicting a human, such as by way of example only and not by way limitation, an injury to an ankle that requires healing, which healing can be facilitated or otherwise encouraged, or, in some embodiments, the brace can simply ensure that the healing process is not detracted or otherwise setback owing to certain actions by the recipient and/or actions that cause a spatial movement of the foot and/or ankle that can have deleterious results with respect to the healing regimen for which the brace is being utilized.

Embodiments can include treating grade 2 and/or 3 ankle sprains (lateral ankle sprain, deltoid sprain, high ankle sprain or syndesmosis injury) by applying the brace within 1, 2, 3, 4, 5, 6, 7, 8, 9, 10, 11, 12, 13, 14 or 15 minutes from the time of injury and/or later, and can include applying the brace over the shoe worn by the recipient at the time of injury, and returning to the activity that resulted in the sprain.

The recipient can retain at least 50, 55, 60, 65, 70, 75, 80, 85, 86, 87, 88, 89, 90, 91, 92, 93, 94, 95, 96, 97, 98, 99 or 100% of one or more or all of his/her capabilities (speed, step length for example) wearing the brace as compared to not wearing the brace.

Embodiments can allow players/workers who suffer ankle injuries to remain in the game/remain at work, within minutes or within a quarter of an hour or within a half hour of suffering the injury.

The brace is a completely external ankle brace vis-à-vis the shoe. The brace does not compromise the fit of the shoe worn by the recipient.

Exemplary scenarios of use can include a scenario where a running back suffers a non-operative high ankle sprain. with a normal standard walking boot or an internal brace, the running back would be unable to practice or play 45 or six weeks. Conversely, utilizing the braces disclosed herein, the running back would be able to practice and/or play within 3 to 4 weeks. Another exemplary scenario entails an offensive tackle suffering an ankle sprain and again. Without the teachings detailed herein, the player would be unable to return for three series with the utilization of an internal ankle brace, which would require a larger shoe. Further, the player will be effective upon his return. Conversely, utilizing the teachings detailed herein, the player is able to return the next series with the braces herein. The player can be confident and effective. Moreover, the utilization of the brace will encourage practice in other words facilitate practice, as it provides a prophylactic to further injury. Coaches and/or players will engage in more drills if not all normal drills because of the prophylactic nature of the device, or, more accurately, because they know of the prophylactic nature of the device.

In an embodiment, the braces herein return a person to at least effective full functionality at least 2, 3, 4, 5 or 6 times faster than a walking brace. (Herein, any statement regarding a walking brace corresponds to a statement about an external brace, and visa-versa, for purposes of textual economy, unless otherwise noted.

Embodiments can provide for a relatively light weight, sleek (reducing the likelihood of tripping/contact of the other foot with the brace), allowing for a return to normal daily activities and/or tasks faster without any deconditioning.

Some exemplary performance features associated with at least some exemplary braces according to some embodiments (the TayCo brace) as compared to other prior control embodiments, such as the walking boot and/or Breg and/or Aircast, which are not embodiments of the teachings detailed herein, exist. Below we present some details associated with the study that resulted in the findings. We also present some exemplary embodiments of some exemplary braces according to the embodiments detailed herein, as well as braces that are different than those embodiments. At least some exemplary embodiments do not have any one or more of the features of the non TayCo brace detailed below. The Long TayCo refers to the embodiments detailed above after FIG. 13 in that the uprights were longer that the Short TayCo (e.g., at least 0.5, 0.75, 1, 1.25, 1.5, 1.75, 2, or 2.25 inches longer as measured from the very bottom with the same size shoe), concomitant with embodiments above, and the Short TayCo refers to the device of FIGS. 1-13. On both the short and the long TayCo, the bottom portion was the same as the embodiment of FIGS. 1-13.

Ankle stiffness was measured in inversion and eversion using a custom fixture and an MTS 8500 load frame. The foot-ankle-tibia model was mounted horizontally in the load frame. For the unbraced condition and for the TayCo braces, a shoe was placed on the foot, and the shoe gripped against a foot plate. A ¼″ rod was placed through the foot plate fixture and the posterior portion of the shoe sole to further prevent motion relative to the fixture. For the walking boot, the boot was bolted to the foot plate. For all tests, an axial load of 37 lbs. was applied to the foot-ankle-shank complex via a rope and pulley. FIGS. 81-84 show the testing devices, etc. FIG. 81 shows an anterior view of test configuration for eversion of the left ankle with a long TayCo brace, FIG. 82 shows the posterior view of the same setup, FIG. 83 shows weights on a cord were used to apply an axial load to the tibia during testing through a pulley system and FIG. 84 shows the walking boot bolted to the foot plate to maintain a rigid connection.

In the testing, the straps of the TayCo brace were pulled very tightly around the ankle, tibia, and foot. The bladder of the walking boot was inflated, but no control over the amount of inflation was used. The inflation was the same for all tests. The distal end of the tibia was displaced vertically (lateral or medial relative to the foot), and the force and displacement were recorded at 100 samples per second. A total displacement of 1 inch was set in order to achieve linear-force displacement measurements without damaging the foot model or the braces. FIG. 85 shows the graphical results, where the load displacement data was collected from the material testing system, and the loading curve is linear, while the unloading curve is nonlinear, reflecting the viscoelasticity of the foam limb. FIG. 86 shows the limb in the deflected position.

Inversion and eversion tests with no brace, the two TayCo braces (short and tall—the tall having some of the features of the embodiments after FIG. 13, more on this in a moment), the CAM (controlled ankle motion) walking boot, a Breg Ultra brace, and an Aircast air-stirrup ankle brace (all control devices available from the manufacturers as of January 2000) were performed with the foot in 0° of flexion. For all tests, an axial load of 37 lbs. was applied to the leg model via a rope and pulley (see FIGS. 87-90). During testing, the straps of the braces were pulled very tightly, and the shoe was tied very tightly around the leg model. For the unbraced condition, Breg brace, and Aircast, ¼ inch lag screws were inserted through the sole of the shoe and into the heel of the foot to minimize motion of the foot within the shoe (see FIG. 91). A ¼″ rod was inserted through the foot plate fixture and the posterior portion of the shoe sole to further prevent motion relative to the fixture. For the CAM walker, the boot was bolted to the foot plate near the heel and ball of the foot. The bladder of the walking boot was inflated, but no control over the amount of inflation was used; the inflation was the same for all tests. More particularly, FIG. 87 shows an anterior view of test configuration for eversion of the left ankle with a long TayCo brace, FIG. 88 shows a posterior view of the same setup, FIG. 89 shows weights on a cord were used to apply an axial load to the tibia during testing through a pulley system and FIG. 90 shows the walking boot was bolted to the foot plate to maintain a rigid connection. FIG. 91A shows side, back, and bottom view of screws through the shoe and into the heel.

Inversion measurements were also performed with the foot in 20° plantar flexion with no brace and with the Long TayCo brace FIG. 91B, which shows, respectively, an anterior view of the test configuration with the ankle in 20° of flexion, a top view of the same configuration, and a posterior view.

For all tests, the force displacement curves were highly linear. The slope of the cure was determined by linear regression, resulting in measurement of the applied force per inch of displacement. The distance from the ankle joint to the point of load application was 15 inches, and the force was converted to a moment by multiplying the force by the 15 inch moment arm. The displacement was similarly converted to an angle using the approximation sin(q)>>q (in radians) for small angles. The total angle of ankle version was less than 4°, for which the error in this approximation is less than 0.004°. Because the foam bone ankle is only a representation of a true ankle, the best measure of the effect of the braces is to determine the difference in stiffness between the unbraced ankle and the braced conditions. Since all of the force-displacement relationships were linear, the principal of superposition applies, and the contributors to the stiffness can be decomposed additively.

With respect to inversion, the walking boot provided the greatest contribution to stiffness in inversion, followed by the long TayCo brace and the short TayCo brace (FIG. 92—showing the walking boot increased the resistance to inversion by the greatest degree—both TayCo braces increased the inversion resistance by twice as much as the Breg or Aircast). The walking boot contributed 56.6% greater inversion resistance than the short TayCo brace and 18.2% more than the long TayCo brace. The long brace provided 32.5% more resistance than the short brace.

For eversion, the long TayCo brace provided the greatest resistance to eversion. The contribution to the eversion resistance was 54.7% higher than the walking boot, and nearly twice as high as the short TayCo brace. The walking boot contributed 26.3% greater eversion resistance than the short TayCo brace (FIG. 93—the long TayCo brace increased the resistance to eversion by nearly twice as much as the short TayCo brace. The walking boot was similar to the short TayCo brace. The Breg and Aircast braces provided much lower support in eversion).

With respect to plantar flexed foot, only the long TayCo brace was tested with a plantar flexed foot, because the walking boot does not allow flexion of the ankle. The three locking screws were removed from the brace to allow flexion. The foot was in 200 plantar flexion, and the tibia was displaced medially to the ankle. In this configuration, the brace increased the inversion resistance of the ankle by 0.9404 N-m/deg (FIG. 94—the TayCo brace increased the inversion resistance of the ankle by 0.94 N-m/deg.). This was a 14.2% increase in stiffness from the unbraced ankle.

The data indicate that the long TayCo brace is much more effective at resisting ankle version than the short brace. It is comparable to a walking boot. The advantage over the walking boot is the ability to allow flexion of the ankle by removing the three locking screws. The results represent the stiffness of the construct under a reasonably high axial load, but lower than body weight and much lower than the force applied at heel strike during walking or running. The results are also consistent with low testing rates and loads. Higher loads could fracture the brace. The TayCo brace outperformed the walking boot in eversion. However, eversion is an uncommon injury mechanism. After an ankle sprain, the lateral ligaments are likely to be injured, and additional support is needed to resist inversion of the ankle. The injury limit of the ankle is most often defined by 30° to 40° of inversion, rather than applied moment (1). This reflects the limits on stretching of the ligaments. While different individuals may have ligaments of differing cross-sections and, therefore, stiffness, the maximum extension of ligaments is similar for all individuals. The moment resisted by the ligamentous structures at this point approaches an asymptote, reaching about 10 N-m (2). For all of the brace constructs tested, the resisting moment of the brace would exceed 10 N-m at approximately 10° of either inversion or eversion. The use of the foam foot/ankle/shank model complicates direct interpretation of the mechanics of the braced ankle.

Measurements of cadaver ankles suggest that the ankle has almost no resistance to inversion/eversion for up to 5° of motion (2). The moment at 10° of inversion is less than 25 kg-cm (2.45 N-m), and that in eversion is only slightly higher. This is predicted to be replicated in vivo due to the time required for the inversion/eversion muscles to fire during foot plants or landing from a jump (3). In contrast, the foam model had a resisting moment of approximate 20 N-m at 3.8° of version in either direction. However, given the stiffness of all of the braces, the resistance to version would increase much more rapidly than the unbraced ankles. In the case of the short TayCo brace, the moment borne by the brace increases at 1.115 N-m/deg, and would exceed the contribution of the ankle ligaments at only a few degrees of version in either direction (2). It is likely that the bending resistance increases more rapidly as the angle increases, but this was not tested to avoid damage to the braces and the artificial ankle. Some error was unavoidable. The testing fixture had finite stiffness. An estimate suggests that in the worst-case scenario, about 10% of the deflection may come from the fixture. However, when the results are converted to moment/degree, and the stiffness of the unbraced leg is subtracted, the resulting stiffness should represent the incremental stiffness of the brace within this linear range. That is the stiffness of the foam foot-ankle-shank complex and the stiffness of the fixture are captured in the measurement with no brace. These can be subtracted to understand the additional contribution of the brace, because the load-displacement curves were linear. An additional source of error was potential motion between the shoe and foot plate. Visual observation indicated that this was minimized under the applied axial load, but it is an unmeasurable error. The walking boot was firmly bolted to the foot plate, but some motion may still occur due to flexion of the plastic on the sole of the boot.

Embodiments of the TayCo External Ankle Brace can also provide a viable lightweight alternative (typically 12 ounces—embodiments of the exemplary braces can be less than and/or equal to 6, 7, 8, 9, 10, 11, 12, 13, 14, 15, 16, 17, 18, 19, 20, 21, 22, 23, 24, 25, 26, 27, 28, 29 or 30 ounces or any value or range of values therebetween in 0.1 ounce increments) to the control cast boot (which can range from two to four pounds). In contrast to cast boots can cause leg length discrepancies that can lead to secondary back, hip and knee problems, embodiments of the brace herein avoid and/or reduce the occurrence of such. The TayCo brace allows a much quicker functional recovery for work, shopping, playing, etc., relative to the control cast boot. In an exemplary embodiment, the recovery times are shortened by at least and/or equal to 10, 15, 20, 25, 30, 35, 40, 45, 50, 55, 60, 65, 70, 75, 80, 85 or 90% or more or any value or range of therebetween in 1% increments relative to the control cast boot.

Some embodiments explicitly exclude the use of the TayCo External Ankle Brace for conditions such as spasticity, severe dropfoot and/or severe ankle deformity.

Some embodiments include embodiments that result in an increased in compliance relative to the control cast boot. By way of example only and not by way of limitation, or on a apples to apples compliance comparison evaluation, compliance over a given recovery time is at least 20, 30, 40, 50, 60, 70, 80, 90, 100, 125, 150, 175, 200, 250, 300, 350 or 400% or more or any value or range of values therebetween in 1% increments over the control cast boot.

In an exemplary embodiment, utilizing the braces according to the embodiments detailed herein, an occurrence of limb length discrepancy causing pain in back, knee, and/or hip is reduced and or eliminated as compared to the control boots, by an amount that is at least and/or equal to 10, 15, 20, 25, 30, 35, 40, 45, 50, 55, 60, 65, 70, 75, 80, 85 or 90% or more, including 100%, or any value or range of therebetween in 1% increment.

In an exemplary embodiment, utilizing the braces according to the embodiments detailed herein, an occurrence of severely altered Gait (resulting in decreased balance and increased energy expenditure) is reduced and or eliminated as compared to the control boots, by an amount that is at least and/or equal to 10, 15, 20, 25, 30, 35, 40, 45, 50, 55, 60, 65, 70, 75, 80, 85 or 90% or more, including 100%, or any value or range of therebetween in 1% increment.

Embodiments can correspond to a total weight of the boot that is less than or equal to 0.25, 0.5, 0.75, 1, 1.25, 1.5, 1.75 or 2 pounds or any value or range of values therebetween in 0.01 pound increments.

In an exemplary embodiment, the braces detailed herein are workboot and/or workshoe compliant.

In an exemplary embodiment, utilizing the braces according to the embodiments detailed herein, an occurrence of slip and/or fall is reduced an or eliminated as compared to the control boots, by an amount that is at least and/or equal to 10, 15, 20, 25, 30, 35, 40, 45, 50, 55, 60, 65, 70, 75, 80, 85 or 90% or more, including 100%, or any value or range of therebetween in 1% increment.

In an exemplary embodiment, utilizing the braces according to the embodiments detailed herein, satisfaction of users of the braces detailed herein, which is a proxy/latent variable for compliance, as compliance is increased when patients are happy, is increased as compared to the control boots, by an amount that is at least and/or equal to 30, 50, 75, 100, 150, 200, 250, 300, 400 or 500 percent or more, or any value or range of therebetween in 1% increment.

Embodiments of some braces, at least, provide for devices that are functional, as compared to the control braces/boots, which are nonfunctional, and thus limit patients returning to normal daily activities and can cause muscle atrophy. In an exemplary embodiment, an occurrence and/or holistic amount of muscle atrophy, utilizing the braces according to the embodiments detailed herein, is reduced and or eliminated as compared to the control boots, by an amount that is at least and/or equal to 10, 15, 20, 25, 30, 35, 40, 45, 50, 55, 60, 65, 70, 75, 80, 85 or 90% or more, including 100%, or any value or range of therebetween in 1% increment.

At least some exemplary embodiments provide a fixed to ROM Conversion, decreasing a likelihood of long term functional impairment. In an exemplary embodiment, utilizing the braces according to the embodiments detailed herein, an occurrence of long term functional impairment is reduced an or eliminate as compared to the control boots, by an amount that is at least and/or equal to 10, 15, 20, 25, 30, 35, 40, 45, 50, 55, 60, 65, 70, 75, 80, 85 or 90% or more, including 100%, or any value or range of therebetween in 1% increment.

In an exemplary embodiment, utilizing the braces according to the embodiments detailed herein, stability is increased as compared to the control boots/braces, by an amount that is at least and/or equal to 30, 50, 75, 100, 150, 200, 250, 300, 400 or 500 percent or more, or any value or range of therebetween in 1% increment.

In an exemplary embodiment, again as noted above, one or both of the lower straps 26 are adjustable. In at least some exemplary embodiments, there is also a method of adjusting the fit of the brace so as to accommodate not only the upper shape of the user's shoe, which can be executed by adjusting strap 30, but also adjusting the fit of the brace so as to accommodate the lower shape of the user's shoe. This can provide utilitarian value beyond that which would be the case if only the upper strap 30 could be adjusted. In an exemplary embodiment, the lower portions of the lateral side wall 18 and medial side wall 16 can be flexed in with tightening of the lower straps 26. This is distinct from the scenario where, for example, only the upper strap is adjusted so as to flex the upper side wall 18. In an exemplary embodiment, the adjustments of the lower strap 26 enables/causes the lower portions of one or both of the sidewalls to flex inwardly a distance that is at least and/or equal to 1, 2, 3, 4, 5, 6, 7, 8, 9, 10, 11, 12, 13, 14, 15, 16, 17, 18, 19 or 20% or more or any value or range of values therebetween in 0.1% increments greater than that which would be the case with only the adjustments of the upper strap, where the baseline is the amount of movement of the sidewalls without the ability to adjust the strap, all other things being equal (for example utilizing the same shoe). In an exemplary embodiment, the aforementioned distance constitute a line distance from the closest points of the bottom of the sidewalls at the location of the bottom straps (e.g., center thereof). This can have utilitarian value with respect to providing a more snug fit overall, but also with respect to the bottom of the shoe. In an exemplary embodiment, this can enable the sidewalls to contour to the shoe more closely than that which would be the case in the absence of the ability to adjust the bottom strap. By way of example only and not by way of limitation, the total amount of area of the sidewalls and the heel enclosure (the entire U-shaped body of the rigid heel enclosure) that is in contact with a shoe is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35% or more or any value or range of values therebetween in 0.1% increments above that which would be the case in the absence of the ability to adjust the bottom strap, all other things being equal.

In an exemplary embodiment, by way of example only and not by way limitation, the total amount of pressure applied by the sidewalls and the heel enclosure (the entire U-shaped body of FIG. 3) that is applied to the shoe is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35% or more or any value or range of values therebetween in 0.1% increments beyond that which would be the case in the absence of the ability to adjust the bottom strap, all other things being equal. In an exemplary embodiment, by way of example only and not by way limitation, the total amount of friction force as measurable in the direction from the heel to the toes (the long axis of the foot) applied by the sidewalls and the heel enclosure, again the entire U-shaped body of FIG. 3) that is applied to the shoe is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35% or more or any value or range of values therebetween in 01% increments beyond that which would be the case in the absence of the ability to adjust the bottom strap, all other things being equal.

For the aforementioned comparisons, the strap that is not adjustable is a strap that, combined with the other portions of the brace, including the adjustable strap of the top, provides for the ability to conveniently fit the brace onto a shoe and then permit the adjustments to enable utilitarian use of the brace. In an exemplary embodiment, the control strap, which is not adjustable, has a total length as measured from the bottom edges of the sidewall (it can be overall longer, but this is the dimension that is measured), of 2.5, 2.75, 3, 325, 3.5, 4, 4.25 or 4.5 inches or any value or range of values therebetween in 0.01 inch increments. The adjustable strap would be able to be tightened so that those values would be lower when the adjustable strap is adjusted in at least some exemplary embodiments.

In this regard, in an exemplary embodiment, with respect to the custom fitting guides detailed herein, it is to be understood that some exemplary embodiments include the action of adjusting the bottom strap in addition to any of the adjustments of the other straps disclosed herein. That is, by way of example only and not by way limitation, there are exemplary methods of fitting the brace, as detailed herein, that include the additional action of making one or more adjustments to the straps that go beneath the shoe or otherwise the lower straps. In this regard, in some exemplary embodiments, the lower straps are adjusted along with the upper strap in the overall fitting process.

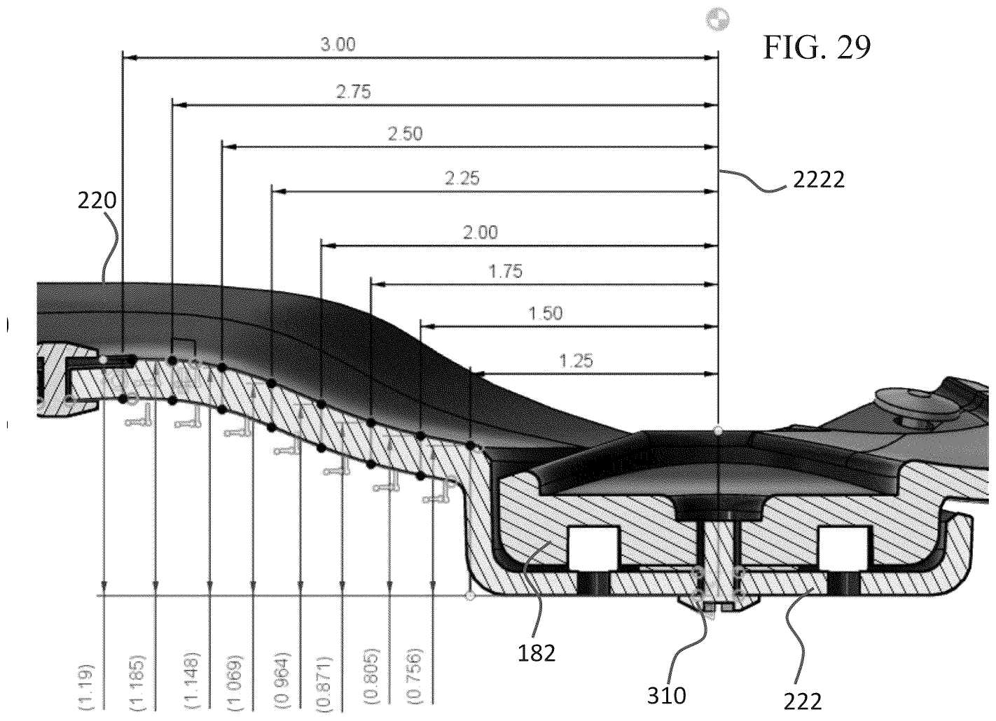

While the embodiment seen in FIG. 29 utilizes a belt strap arrangement that has holes that permit adjustment in digital increments, in other embodiments, an analog arrangement can be utilized. Indeed, in an exemplary embodiment, Velcro attachments can be utilized.

In view of the above, it is noted that at least some exemplary embodiments include methods of fitting or otherwise adjusting otherwise establishing the brace(s) disclosed herein for use for a particular user or patient. In an exemplary embodiment, a healthcare professional sets the mode of operation of the hinge according to the desired utilitarian features associated there with. In some exemplary embodiments, a patient's ankle should be fully immobilized. Thus, in an exemplary embodiment, the immobilization lock arrangement will be set during the method of fitting. Conversely, some patients can accept a limited range of motions the, and thus the limited range of motion note will be set by the healthcare professional during the method of fitting. That said, while we are making reference to fitting, it is noted that some other embodiments are directed towards methods of adjusting the brace. In this regard, any disclosure herein of a method of fitting corresponds to a disclosure of an alternate embodiment of a method of adjusting the brace after fitting.

And in this regard, it is noted that while in some exemplary embodiments, the method of fitting includes setting the mode of operation of the hinge for limited range of movements, in other embodiments, the patient returns back to the healthcare professional, and after an evaluation, the healthcare professional determines that the patient can graduate from the immobilized mode to the limited range of motion's mode. In some other embodiments, this determination can be made by telemedicine for example, and the patient/recipient can adjust the modes on his or her own.

In some exemplary methods include a method of fitting and/or a method of adjustment where the mode is set at a full range of motion. This can be done at the outset and/or can be adjusted after the brace has been used in the patient has experienced healing. In note also that in some embodiments, the mode of operation can be adjusted from full movement to immobilization and/or from limited movement to immobilization and/or from full movement to limited movement, etc. For example, it could be that the healing process is not progressing in a desired manner, and/or that the initial prognosis that motion (full motion or limited motion) was permissible is no longer the case. Thus, the mode could be “tightened” to reduce and/or eliminate the amount of motion that the brace will permit.

In note also that some embodiments include methods where the adjustments through the modes are executed within the same day and/or within hours of each other. By way of example only and not by way of limitation, the limited range of motion and/or the full range of motion can be utilized while the patient is driving, and then the immobilization can be used thereafter owing to the scenario where it is deemed less likely that the patient is going to injure himself or herself while driving as compared to nondriving activities. Alternatively, and/or in addition to this, the range of motions can be set for a particular leg based activity. If the patient playing some form of sports for example, an adjustment could be made for that particular sport, and then in adjustment could be made for walking.

Embodiments can utilize locking bars to adjust the modes of the hinges. By way of example only and not by way limitation, one or two or more locking bars can be utilized. In an exemplary embodiment, there is a first locking bar that provides for complete immobilization, and then a second locking bar that has a different configuration that provides for the limited range of motion when utilized instead of the first locking bar. When the locking bars are removed/there is no locking bar, the full range of motion/free motion can be achieved. It is noted that in some embodiments, there are different receivers/receptacles in the hinge apparatus to receive the respective hinge bars. For example, with respect to the center of rotation, there could be an upper bar and a lower bar, where the upper bar corresponds to the first locking bar in the lower bar corresponds to the second locking bar. Still further by way of example with respect to the center of rotation, there can be a forward bar and a rear bar, where the forward bar corresponds to the first locking bar in the rear bar corresponds to the second locking bar. Combination of these can exist where, for example, there is only a rear bar and a upper bar or a rear bar and a lower bar, etc. Each bar when utilized provides for different limitations of motion. In an exemplary embodiment, it can be a result of the geometry of the given locking bar, where the first locking bar is different than the second locking bar with respect to size and dimension. Conversely, in an exemplary embodiment, it is a function of the receptacle, where a single locking bar having a single design with respect to size and dimension can be utilized to achieve the different limitations, where if, for example, the locking bar is utilized in the upper receptacle, the range of motion can be limited, and if that locking bar is instead moved to the bottom receptacle, the range of motion is completely immobilized.

And moreover, different types of locking bars can be utilized to achieve the aforementioned limitations. By way of example only and not by way limitation, a flexible locking bar can provide for the limited range of motion, or more accurately, and permit the limited range of motion, while he stepped locking bar can prevent the motion/enable the immobilized state of the hinge assembly.

Other arrangements can be utilized to provide for the different modes of operation of the hinge. And while the embodiments disclosed herein have been presented in terms of having three distinct modes, and some embodiments, there can be more or less modes. Indeed, in an exemplary embodiment, the aforementioned limited range of motions can be adjustable: the hinge assembly can be configured to “dial a range” for example so that the dorsiflexion angle and/or plantar flexion angle can be adjusted as deemed utilitarian.

Note also that in at least some exemplary embodiments, the locking bars can be configured to provide different range of motion limitations period, for example, there can be sets of locking bars that enable the aforementioned 5° of forward movement and 10° of rearward movement, and then other sets of locking bars that enable for example, 7° of dorsiflexion and 8° of plantar flexion, by way of example. Thus, embodiments include kits that can be provided to a healthcare professional, that will enable the healthcare professional to make adjustments to the angles of motion depending on the deemed utilitarian value thereof by the healthcare professional. Embodiments also include the ability to enable the ultimate user to adjust these features.

Embodiments can enable the utilization of a single brace for two, three, four, five, six or more pairs of shoes that an individual user may own. Accordingly, methods include the utilization of a brace with a first pair of shoes, and then with a second pair of shoes. Methods include the utilization of a brace with a friend her shoes, a second pair of shoes, a third pair shoes, a fourth pair shoes, etc., with issues are utilized in interleaved manner, such as for example, the first pair, the second pair, then the first pair again and then a third pair, and then a fourth pair, and then the third pair again, and so on. It is noted that the phrase pair shoes refers to the fact that a user will be wearing a pair of shoes, but of course the brace will be only utilized with one of the two shoes. The point is that the brace enables the utilization of the same pair of shoes, as opposed to having to have, for example, two shoes respectively having different sizes, one of the sizes accommodating the brace. That is, in at least some exemplary embodiments, the shoes are the same size, albeit for different left right feet.

An exemplary embodiment enables a generic brace to be customized for a particular person at the time of fitting. In an exemplary embodiment, a generic brace can be fitted to any of a 30, 35, 40, 45, 50, 55, 60, 65, or 70 or any value or range of values therebetween in 1% increments (e.g., 33 percentile, 66 percentile, 37 to 66 percentile) percentile human factors engineering male or female within 2, 3, 4, 5, 6, 7, 8, 9, 10, 11, 12, 13, 14 or 15 minutes or any value or range of values therebetween in 10 second increments, along with adjustments to the bottom strap to achieve any one or more of the after mentioned fitting features detailed herein. Thus, in an exemplary embodiment, a generic brace can be fitted to a wide variety of human beings in a fast manner. And it is noted that the aforementioned human factors engineering values correspond to the resulting shoe size thereof. That is, the after mentioned values correspond to such humans wearing pertinent shoes for such humans.

It is noted that there is a difference between a custom fit and a custom fabrication. At least some exemplary embodiments disclosed herein explicitly exclude custom fabrication. In this regard, some embodiments enable such utilitarian flexibility with respect to custom fitting that custom fabrication is not needed. This as distinguished from, for example, some prior art boots and the like.

In an exemplary embodiment, the customization entails removing material. In an exemplary embodiment, the customization explicitly excludes adding material. In an exemplary embodiment, the customization entails applying heat to one or more of the components and/or adjusting a state of one or more of the components. By way of example only, one or more other components of the brace can be heated to enable plastic deformation, and then upon cooling, the deformation established is retained.

In any event, at least some exemplary embodiments utilize components that are made from materials that are easily removed, such as, for example, with an exacto knife or the equivalent or with a handheld grinder, such as a Dremel™ tool. Embodiments can be such that sandpaper can be utilized to remove portions of the brace. Any arrangement of removing material that can enable the utilitarian value of customizing the brace for fitting can be utilized in at least some exemplary embodiments.

In an exemplary embodiment, the structural components and/or the entire brace is made of polymer components. In an exemplary embodiment, elements 22 and 20 and 16 and 14 and 10 are all made of polymers. In an exemplary embodiment, the hinge is also made of a polymer. In an exemplary embodiment, straps are made of polymers, albeit a more flexible polymer. Accordingly, in some exemplary embodiments, there is an all plastic and/or all polymer brace.

In some embodiments, the polymer structures can be plastically deformed. This is in addition to the elastic deformation which may be present. And to be clear, in an exemplary embodiment, the fitting methods herein are executed by elastically deform in the various portions of the structure of the brace. Still, in some embodiments, such as for a given individual, at fitting, plastic deformation of one or more of the structural components can be controllably executed to provide for a more utilitarian fit beyond that which would otherwise be the case without the plastic deformation.

The above said, referring to the locking pins detailed above, at least some exemplary embodiments utilize metal locking pins. That said, in at least some exemplary embodiments, the metallic portions thereof can be embedded or otherwise covered with the polymer.

FIG. 97 presents another external ankle brace of the present disclosure indicated as ankle brace 500. Here, for simplicity, a rear view of the brace 500 is shown. The external ankle brace 500 includes a monolithic body (what is shown minus element 28) including a rigid heel enclosure 1000, a lateral upright extension 2000, a medial upright extension 2200, a lower fastening system 24 (not shown, but as disclosed herein and variations thereof, such as the other lower fastening systems detailed herein—again, any embodiment can be substituted with another embodiment unless otherwise noted) and an upper fastening system 28. The rigid heel enclosure 1000 is monolithic with the uprights 2000 and 2220 in this embodiment. In an exemplary embodiment, the uprights and the rigid heel enclosure can have the same basic features and shapes as detailed above at least with respect to how those components interface with the shoe and the body of the human. In an embodiment, from the outside, the components can be identical to those detailed above, with the exception that the uprights and the heel enclosure are monolithic/there is no joint. In some embodiments, the exception is even less so, in that a faux joint is present to give the appearance that the external ankle brace is the more higher end model with the joints where the uprights can move/rotate relative to the rigid heel enclosure. Conversely, at least from the external side (and both in other embodiments), the uprights can seamlessly blend in with the heel enclosure, such as that shown in the embodiment of FIG. 97, consistent with an embodiment where the monolithic body is molded in one fell swoop to create the monolithic external ankle brace body. The body can have the foam padding and the fastening systems, etc., detailed herein, added thereto in subsequent manufacturing operations. In this regard, any one or more of the teachings herein can be implemented in a monolithic body as described.

FIG. 98 presents another exemplary embodiment of an external ankle brace 501, with like reference numbers corresponding to the teachings herein. Here, the upright 2001 and the upright 2221 on the opposite side are monolithic with the rigid heel enclosure 1001, establishing a monolithic body. However, the above-noted reinforcing strut, here, with respect to reference 1287, is located on the outside of the monolithic body and attached thereto utilizing screws or rivets, etc. As seen, the heel enclosure 1001 includes a rear portion 1201, and sidewalls 1800 and the opposite side sidewall (eclipsed by the shoe), all parts of the monolithic body.

FIG. 99 shows a monolithic body 5002 for an external ankle brace according to an embodiment. None of the additional attachment components that make up the full external ankle brace are shown. Here, the body 5002 includes uprights 20022 and 22222, and the rear portion 12022 and the sidewalls 18022 and 16022. This body is formed in an embodiment by injection molding and the resulting product is a monolithic body as shown.

Embodiments of the monolithic body of the arrangements of FIGS. 97, 98, and 99, can have any one or more or all of the features detailed above providing that the art enable such, other than of course the ability of the uprights to rotate relative to the heel enclosure. In some embodiments, the dimensions of the monolithic body components are basically the same the embodiments of the rotating components, save for the interface between the uprights and the heel enclosure/components that enable the rotation. Again, embodiments can give the visual appearance of a rotating embodiment from a distance (a close inspection will reveal the monolithic nature of the components—depending on the mold technology, faux gaps can be provided to give the appearance of the rotating arrangements). Accordingly, embodiments can include taking any one or more the embodiments detailed above, but modified so that the uprights are monolithic with the heel enclosure. Indeed, in embodiments, some of the joint components, such as the Chicago screw, can be present, it is just that the uprights cannot rotate relative to the heel enclosure because of the monolithic nature thereof.

And in some embodiments, it could be that only one of the uprights is monolithic with the heel enclosure. For example, the lateral upright can be monolithic with the heel enclosure, or the medial upright can be monolithic with the heel enclosure. The opposite can have the joint or otherwise can have the rotatable features detailed above.

Note also that in an embodiment, a forward or rearward connecting portion can extend between the lateral and medial uprights in the front and/or in the back, which can be monolithic with the uprights. This can be a single “strap like” component or can be two or three or more (front or back—the front need not be the same as the back). FIG. 100 shows an exemplary embodiment of this embodiment, where connecting portion 5115 is monolithic with the uprights as can be seen. In an embodiment, the body 5009 comes out of the mold in this manner (the upper fastening system 28 is not part of the body, but is attached after molding). Embodiments can also include a forward connecting component that is monolithic with the uprights. Note that while the embodiment shown in FIG. 100 has the connecting portion extending monolithically from both of the uprights, in an embodiment, the connecting portion can extend only from one of the uprights. In an embodiment, a fastening device such as a screw or a Velcro strap or a set fastener system for example can be utilized to connect the free end of the connector to the upright. In an embodiment, this can make the molding of the monolithic body where the forward and rear connectors are monolithic with one or more of the uprights easier because there is not a closed body to be addressed with respect to the mold surfaces. Accordingly, embodiments can dispense with the above-noted wraparound straps or the like, and/or can include arrangements where there is only a strap and the front or the back, which strap does not wrap completely around the calf or lower leg of the user. Instead, the arrangement can correspond to the upper fastening system or the lower fastening system except that it extends more horizontally than vertically as opposed to those fastening systems. That said, in an embodiment, a wraparound strap can be utilized over the connector portions. The connector portions could have portions that can flex or otherwise accommodate the cinching of the wraparound strap.

And corollary to the concept of the connector portion/connector component being monolithic with the uprights, FIG. 101 shows body 5003 which is an embodiment where the lower fastening system is monolithic with the sidewalls. Here, there is a connector component/connector portion 10260 which is monolithic with the sidewalls 1802 and 1602. It is formed in the same molding process that is used to form the sidewalls and the uprights in an exemplary embodiment. Accordingly, in an embodiment, instead of the above-noted strap which is riveted to both of the sidewalls, the connecting component monolithic with the sidewalls. In an embodiment, the connecting component can have the sufficient features to enable the connecting component to be walked on. When viewed from the front or the back, the connecting component would look like a very wide and short “U” portion, with the tops of the Us being monolithic with the sidewalls. Also, this concept can be applied to the upper fastening system, as seen in FIG. 102, which shows body 5004, where a connector portion 10230 extends from the top of the sidewall 1802 to the top of the sidewall 1602, with the connector portion 10320 being a monolithic part of the body 5004. In this exemplary embodiment, the external brace is sized and dimensioned so that the user shoe can fit into the heel enclosure but still remain sufficiently snug. In an embodiment, a spacer/buttress can be fit between the top of the shoe and the component 10230 (and can be secured by a strap around the component 10239 for example) that will create a “wedge” and secure the arrangement if needed. Still, embodiments can be such that the shoe can be “forced” into a snug enclosure because the body has a modicum of flexibility.

In this embodiment, the connector portion 10230 is not adjustable. However, in an embodiment where, for example, one end of the connector portion is free and not attached or otherwise not connected to the given sidewall, an adjustable arrangement can be implemented. Indeed, any of the securement mechanisms detailed above or otherwise the mechanisms that enable adjustment can be utilized for this embodiment. Also, a Velcro system or a snap system etc. can be utilized. That is, as with the bottom component 10260, the top component 10230 can be connected to one side and not the other, and the free end can be secured to the other by any of the manners detailed herein for securing the upper fastening system or the lower fastening system for that matter. Again, such an embodiment permits the body to be more easily made in an injection mold. And while embodiments have focused on the idea of the connector portion 10230 being monolithically directly attached to only one sidewall, in an embodiment, the connector portion 1230 can be bifurcated and can extend from each of the sidewalls to a central location, where, at that central location or offset from one side or the other, a connector regime can be utilized to connect the ends together, such as with a snap coupling or a Velcro strap, etc.

Note further that in an embodiment, the upper fastening system and the lower fastening system can instead be a monolithic component or include a monolithic body. By way of example only and not by way limitation, in an exemplary embodiment, a varying radius ring (i.e., a non-circular ring) body can be used and slipped over the front of the brace so as to provide a combined fastening system. The varying radius ring can be sized and dimensioned to be custom for a user's shoe, or can be of standard sizes. One size can be used for one size brace, or two or three or four or more sizes can be provided with a one size brace, so as to provide the desired snugness. In an exemplary embodiment, this varying radius ring is fit over and around the sidewalls and pushed backwards towards the ankle. This will provide a constriction on the sidewalls and push the sidewalls towards the shoe or otherwise hold the sidewalls against the sides of the shoe. In an embodiment, there can be latches or the male protrusion utilized with the lower fastening system by way of example to hold the varying radius ring in place. In an embodiment, the ring can be made of the same material or similar material as the strap of the lower fastening system. In an embodiment, it can be made of the same material or similar material as the heel enclosure. Any material that can utilitarian value and otherwise implement the teachings detailed herein can be utilized in at least some exemplary embodiments.

Note also that while the embodiment just described is described in terms of a varying radius ring (in the at rest/relaxed configuration), in other embodiments, it can be a circular ring in its relaxed state, and be sufficiently deformable (elastically) that will adopt the required shape for pressing the sidewalls against the shoe.

FIG. 103 shows an exemplary noncircular ring 10360 looking down the longitudinal axis thereof in the ring's relaxed state. In an exemplary embodiment, this is slid over the sidewalls of the body of the brace 5018 as shown in FIG. 105. In an embodiment, the ring 10360 is sufficiently stiff so that its basic shape is retained, such as in an exemplary embodiment where the ring 10360 is custom-made for a given user. In an alternate embodiment, the ring 10360 will flex substantially. In an exemplary embodiment, the ring in use, flexes so that the diameter of the inside of the ring takin at 10 increments does not vary more than 30, 25, 20, 15, 10, 9, 8, 7, 6, 5, 4, 3, 2 or 1% or any value or range arise therebetween in 1% increments from the average diameter for the values over all of those 1° increments of the rings relaxed state when the ring is placed fully on to the body 5018 for use. In an exemplary embodiment that feature is present over no more than or at least 160, 150, 140, 130, 120, 110, 100, 90, 80, 70, 60, 55, 50 or 45 degrees contiguously or any value or range of values therebetween in 1° increments.

Note that the wedge component detailed above can be used with these in some embodiments.

FIG. 104 shows an exemplary embodiment where the ring 10460 is sufficiently flexible so that it will have a modicum of deformation at standard gravity at sea level under 1 atm pressure depending on how it's positioned. In this regard, the embodiment of FIG. 104 is a flexible ring.

Returning back to FIG. 105, it can be seen that the ring 10360 includes holes 10505 that interface with cylindrical protrusions located on the outside of one or more of the sidewalls. In this exemplary embodiment, those cylindrical protrusions are sufficiently flexible and/or the ring 10360 sufficiently flexible to enable the ring to be slid over those protrusions with the human shoe in the body 5018 and then for those protrusions to “snap” into the holes 105052 hold the ring in place when the body 5018 is worn and otherwise utilized. Here, this can enable the combined lower fastening system and upper fastening system into one single ring. In this embodiment, the ring is monolithic and continuous or otherwise a so-called endless ring.

In the interest of additional completeness, FIG. 106 shows the body 5018 without the ring 10360. As seen, the cylindrical studs 10501 are shown on the side of the sidewall. In an embodiment as shown here, there are three studs on each of the sidewalls. In an exemplary embodiment there can be more or less studs, and the number of studs need not be equal on either side.

FIG. 107 shows another exemplary embodiment where a belt and buckle system is utilized to retain the ring 10707, which can correspond to the rings detailed above or variations thereof, by placing the strap 10703 over the outside of the ring and then threading the strap through loop 10701, which can be formed as part of the body when the body is molded, or can be attached thereto after manufacturing of the mold, where the strap 10703 has a whole 10705 to receive a cylindrical protrusion to lock the strap in place. Note that in an alternate embodiment, the cylindrical protrusion could be part of the strap, and then this could be fitted into a hole in the sidewall. Note that this arrangement could also be utilized for the other embodiments of the ring (the cylindrical portion to be part of the ring or otherwise carried by the ring, and there could be a hole in the side wall to receive the cylindrical portion and thus lock the ring in place).

FIG. 108 depicts another exemplary embodiment of the single combined upper fastening system and lower fastening system according to an exemplary embodiment. Here, there is a strap 10807, which can correspond to any of the straps detailed herein or otherwise any of the components that are utilized to span the distance from the lateral side wall to the medial side wall above and below the shoe when worn. The strap can be in the form of a traditional belt, with a belt buckle 10808 having a tong fitting through holes in the belt (the circles not labeled). The belt 10807 can be fitted through loop 10801 which can be monolithic with the body 5018 or can be attached thereto in a subsequent manufacturing operation. The loop 10801 can hold the belt in the fore and aft position so that the belt does not slide off during use, concomitant with the loop of the embodiments detailed above. While a belt buckle arrangement is utilized in the embodiment of FIG. 108, in an alternate embodiment, the Velcro system as detailed above can be used. A modified version of the arrangement for lower fastening system where one side of the strap is detachable from the sidewall can be utilized. Any device system and/or method that can enable the belt 10807 to be tightened or otherwise fitted to the brace so as to provide the compression or the securement features detailed herein can be utilized in at least some exemplary embodiments.

And note that while the embodiment shown present a separate ring or a separate strap, etc., in the combined upper and lower fastening system, in an alternate embodiment, there could be a portion of the body that extends away from the top of one of the sidewalls and also a portion of the body that extends away from the bottom of the sidewall, and these portions could have sufficient length to wrap around to the other side wall and then be attached to one another utilizing a fastening mechanism, such as a buckle arrangement or a Velcro arrangement or the cylindrical protrusion fitting into a hole arrangement. In this regard, the portions would be monolithic with the body of the external brace and would be sufficiently flexible to extend accordingly to provide the securement achieved by the upper fastening system in the lower fastening system. In an embodiment, there could be components on the other side wall that would hold the respective portions in place relative to the sidewall in addition to any coupling between those two portions with each other, or even as an alternative. In an exemplary embodiment, the portions may never touch each other or other wise reach each other with respect to location on the opposite side wall. For example, the bottom portion could extend to the other side wall and then upwards to interface with a fastener, and the top portion could extend to the other side wall and then downwards to interface with another fastener, these fasteners separated from each other or otherwise arranged so that the portions can have a distance where they do not meet each other or otherwise cannot meet each other when the boot is worn but still the utilitarian value of the fastening systems is achieved. The top portion can be individually adjusted relative to the sidewall and the bottom portion can be individually adjusted to the sidewall. FIG. 109 shows an exemplary embodiment of this arrangement, where there is bottom portion 10907A extending from the far side wall to the near side wall with respect to the figure, and also upper portion 10907B extending from the far side wall to the near portion. Both of these are fitted through respective loops 10909, which can be monolithic with the body of the boot, and are fastened or otherwise retained in the taught position using the cylindrical protrusion and hole arrangement in this exemplary embodiment, although a Velcro arrangement can be used, such as where Velcro pads are adhesively bonded to the sides of the protrusion that face the sidewalls as well as to the sidewalls on the sides that face the ends of the portions. And note that this embodiment shows that the directionality of the portions can be different in this embodiment. That is, the portions need not be aligned, although they can be. Concomitant with the teachings above, the bottom portion could be forward or aft of the upper portion. And note also while this embodiment shows only one portion of the top and one portion of the bottom, other embodiments can utilize two or more portions at the top and/or the bottom, and the numbers need not be the same. Corollary to this is that two or more rings can be utilized in at least some exemplary embodiments, where, for example, Wondering, such as the ring that is utilized more forward, we have a smaller overall are average diameter than the other ring, owing to the geometry of the foot by way of example.

And while the embodiments above have presented rings is having a radial extension that is the same with respect to axial distance, other embodiments can include utilizing rings that have a radial extension that varies with respect to the axial distance. In this regard, FIG. 110 shows an exemplary ring 101160, that has a bend as shown. This bend accommodates the fact that the upper fastening system extends at a different angle than the lower fastening system owing to the geometries of the external ankle brace. In this embodiment, the ring can be fitted over the external ankle brace in a manner consistent with the teachings detailed above, and the cylindrical protrusions on the sidewalls 101501 can be fit into holes in the ring or vice versa or a combination of the two, to hold the ring in place during use. Any of the other arrangements detailed herein that can be utilized to hold the ring in place can be utilized.

And while the embodiments disclosed herein have focused on a generally monolithic/uniform material based ring, in other embodiments, the ring can be a multi-material or multicomponent ring. By way of example only and not by way limitation, the bottom could be the more durable and more rigid material of the lower fastening system, and the upper portion could be the more flexible material and more fabric like material of the upper fastening system is disclosed herein, by way of example only. Indeed, in an exemplary embodiment, instead of a ring, the lower fastening system could be a U-shaped body that traps the sidewalls within the legs of the U, and then a Velcro strap can extend from one of the legs to the other on the outside up over the top of the shoe, and this can be utilized to tighten the arrangement. The U-shaped body could be sufficiently flexible that it can be pulled up so that the distance between the legs of the U would be shorter and thus provide compression against the sidewalls. Alternatively, the U-shaped body could be more rigid and it can be custom fit. FIG. 111 shows an exemplary embodiment this by way of example only and not by way limitation, where element 11360A is the U shaped body for the bottom, and element 11360B is the fabric strap Velcroed by way of example to the U shaped body.

Embodiments disclosed herein have focused on a rigid external brace/body therefore. In some embodiments, the brace may not be rigid but instead can be a relatively flexible arrangement. Also, in some embodiments, portions of the body of the brace can be flexible while other portions can be rigid, all relative to one another. By way of example only and not by way of limitation, instead of operates rotating relative to the heel enclosure, the operates could still be able to articulate relative to the heel enclosure because the interface between the operates and the heel enclosure or otherwise the portions thereof proximate one another are sufficiently flexible to enable the articulation. And note also that in an embodiment, stops could be provided so that the articulation can be limited according to the limits detailed above. Still, in an embodiment, it can be that with respect to XAX number of foot-pounds provided to one or both of the uprights, the uprights will articulate by at least and/or no more than 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 degrees or less or any value or range of values therebetween in 1° increments forward and/or backward (and the two need not be the same). In an exemplary embodiment, XAX can be less than, greater than and/or equal to 20, 19, 18, 17, 16, 15, 14, 13, 12, 11, 10, 9, 8, 7, 6, 5, 4, 3, 2, 1.5, 1, 0.9, 0.8, 0.7, 0.6, 0.5, 0.4, 0.3, 0.2 or 0.1 pounds or any value or range of values therebetween in 0.1 pound increments.

That said, even with the rotating embodiments, locked or non-locked and moved to the most forward or rearward position without deformation/flexing the uprights, it can be that with respect to XAX number of foot-pounds provided to one or both of the uprights, the uprights will articulate by at least and/or no more than 20, 19, 18, 17, 16, 15, 14, 13, 12, 11, 10, 9, 8, 7, 6, 5, 4, 3, 2 or 1 degrees or less or any value or range of values therebetween in 1° increments. In an exemplary embodiment, XAX can be less than, greater than and/or equal to 20, 19, 18, 17, 16, 15, 14, 13, 12, 11, 10, 9, 8, 7, 6, 5, 4, 3, 2, 1.5, or 1 pounds or any value or range of values therebetween in 0.1 pound increments.

It is noted that any disclosure of a device and/or system detailed herein also corresponds to a disclosure of otherwise providing that device and/or system and/or utilizing that device and/or system.

It is also noted that any disclosure herein of any process of manufacturing or otherwise providing a device corresponds to a disclosure of a device and/or system that results therefrom. Is also noted that any disclosure herein of any device and/or system corresponds to a disclosure of a method of producing or otherwise providing or otherwise making such.

Any disclosure herein of a device and/or system corresponds to a disclosure of utilizing that device and/or system for the purposes detailed herein. Any disclosure herein of an action that is executed utilizing a device and/or system corresponds to a disclosure of that device and/or system.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

There are both various features disclosed herein associated with various embodiments disclosed herein, as well as various patent applications incorporated by reference herein that respectively disclose respective various features associated with respect to various embodiments of those patent applications. Any one or more or all of the various features referenced in the preceding sentence can be combined with any one or more or all of other various features referenced in the preceding sentence unless otherwise specifically stated and/or unless the art does not enable such combination. Also, any one or more or all of the various features referenced in the beginning sentence of this paragraph can be explicitly excluded from combination with any one or more or all of the other various features referenced in the sentence at the beginning of this paragraph unless otherwise specifically stated and/or unless the art does not enable such exclusion of combination.

Any disclosure herein of a method action corresponds to a disclosure of a related product and/or system that is used in that method action. Any disclosure herein of any device and/or system corresponds to a disclosure of a resulting method of utilizing that device and/or system. Any disclosure herein of a device and/or system corresponds to a disclosure of a method of making that device and/or system. Any disclosure herein of a method action of making a component and/or a device and/or system corresponds to a disclosure of a resulting component and/or device and/or system that results from such method action.

1. An external ankle brace for selectively restricting movement of an ankle in at least one of a first direction and a rotation direction, and selectively permitting movement of the ankle in a second direction, wherein the external ankle brace is disposed on the exterior of a shoe, the shoe having a heel portion, a toe portion longitudinally spaced from the heel portion, a sole, and oppositely disposed sides, the external ankle brace comprising: a rigid heel enclosure having a rear portion and a forward portion, the rear portion configured to receive and at least partially encircle the heel portion of the shoe, the forward portion having a medial sidewall and a lateral sidewall for collectively and concurrently at least partially encircling the sides of the shoe concurrent with the rear portion connecting the medial and lateral sidewalls to collectively at least partially encircle the side, and fully encircle the heel portion, of the shoe, the lateral and medial sidewalls each extending from the rear portion toward a toe of a wearer's foot and each extending beyond a talus of the wearer's foot; a lateral upright extension selectively perpendicular to the rigid heel enclosure and pivotally attached to the lateral sidewall, the lateral upright extension including a lateral reinforcing strut; a medial upright extension selectively perpendicular to the rigid heel enclosure and pivotally attached to the medial sidewall, the medial upright extension including a medial reinforcing strut; a lower fastening system comprising at least one lower adjustable connecting strap for connecting the lateral sidewall to the medial sidewall and extending underneath the sole of the shoe; and an upper fastening system comprising at least one upper connecting strap for selectively connecting the lateral sidewall to the medial sidewall across the top of the shoe, the upper connecting strap being located longitudinally between the lower connecting strap and the lateral and medial upright extensions.

2. The external ankle brace of claim 1, including an upright fastening system comprising at least one upright connecting strap for selectively connecting the lateral upright extension to the medial upright extension above the ankle.

3. The external ankle brace of claim 1, wherein the upper connecting strap is a second upper connecting strap, and the upper fastening system includes a first upper connecting strap for selectively connecting the lateral sidewall to the medial sidewall across the top of the shoe, the second upper connecting strap, the lower connecting strap being located longitudinally between the second upper connecting strap and the lateral and medial upright extensions

4. The external ankle brace of claim 3, wherein the second upper connecting strap is longitudinally wider than the first upper connecting strap.

5. The external ankle brace of claim 1, wherein the lateral and medial reinforcing struts are malleable and configured to accept and maintain a nonplanar shape profile, to at least partially impart the nonplanar shape profile to the corresponding lateral or medial upright extension.

6. The external ankle brace of claim 1, wherein the lateral and medial upright extensions are made of a first material and the lateral and medial reinforcing struts are made of a second material which is more ductile than the first material.

7. The external ankle brace of claim 6, wherein the first material is a polymer and the second material is a metal.

8. The external ankle brace of claim 1, wherein the lateral and medial upright extensions wholly encapsulate the lateral and medial reinforcing struts.

9. The external ankle brace of claim 8, wherein the lateral and medial upright extensions are molded around the lateral and medial reinforcing struts.

10. The external ankle brace of claim 1, wherein the rear portion includes a rear reinforcing strut.

11. The external ankle brace of claim 10, wherein the rear reinforcing strut is malleable and configured to accept and maintain a nonplanar shape profile, to at least partially impart the nonplanar shape profile to the rear portion.

12. The external ankle brace of claim 11, wherein the rear portion is made of a first material and the rear reinforcing strut is made of a second material which is more ductile than the first material.

13. The external ankle brace of claim 12, wherein the first material is a polymer and the second

14. The external ankle brace of claim 10, wherein the rear portion wholly encapsulates the rear reinforcing strut.

15. The external ankle brace of claim 10, wherein the rear portion is molded around the rear reinforcing strut.

16. The external ankle brace of claim 10, wherein the rear reinforcing strut is directly pivotally connected to the lateral and medial reinforcing struts.

17. The external ankle brace of claim 10, wherein the rear reinforcing strut includes a curved rear strut body extending around the heel portion of the shoe and lateral and medial strut stubs extending substantially perpendicularly from the rear strut body, at opposed locations on the rear strut body, the lateral and medial reinforcing struts being directly pivotally connected to the lateral and medial strut stubs, respectively.

18. The external ankle brace of claim 1, including a restraining bolt connected to a chosen one of the lateral and medial upright extensions and operative to selectively restrict pivoting of the chosen upright extension respective to a corresponding lateral or medial sidewall.

19. The external ankle brace of claim 16, including a restraining bolt connected to a chosen one of the lateral and medial reinforcing struts and operative to selectively restrict pivoting of the chosen upright extension respective to the rear reinforcing strut.

20. The external ankle brace of claim 1, wherein the upper connecting strap has a variable length fastening operable for manually adjustment by a wearer to a predetermined length, and the lower connecting strap has a constant length and is not manually adjustable by a wearer.

21-41. (canceled)
